(19) United States
(12) Patent Application Publication
(10) Pub. No.: US 2020/0166513 A1
(43) Pub. Date: May 28, 2020

(54) GLYCAN NODES AS CANCER MARKERS

(71) Applicants: Chad Borges, Avondale, AZ (US); Shadi Ferdosi, Belmont, CA (US)

(72) Inventors: Chad Borges, Avondale, AZ (US); Shadi Ferdosi, Belmont, CA (US)

(21) Appl. No.: 16/681,453

(22) Filed: Nov. 12, 2019

Related U.S. Application Data
(60) Provisional application No. 62/758,026, filed on Nov. 9, 2018.

(51) Int. Cl. G01N 33/574; G01N 30/72

(52) U.S. Cl. CPC G01N 33/57488; G01N 2458/00; G01N 2400/10; G01N 30/7206

(57) ABSTRACT

The present disclosure relates to methods of using plasma and serum (P/S) glycomics based on glycan linkage analysis that captures unique glycan features such as α2-6 sialylation, β1-6 branching, and core fucosylation as single analytical signals to evaluate the behavior of P/S glycans in all stages of lung cancer and across various stages of bladder, prostate, ovarian, and pancreatic cancer.

### FEDERALLY SPONSORED RESEARCH OR DEVELOPMENT

This invention was made with government support under R33 CA191110 awarded by the National Institutes of Health. The government has certain rights in the invention.

### BACKGROUND

### Cancer

Serum glycan composition and structure are well known to be altered in many different types of cancer.¹⁻⁴ In fact, for over a decade now, global blood plasma/serum (P/S) glycomics has held out the promise of new, non-invasive cancer markers derived from a small volume of this easily accessible biofluid.^{5,6} Modern analytical methods for quantifying the relative abundance of different glycans in P/S vary widely⁷⁻⁹, ranging from multiplexed capillary gel electrophoresis with laser-induced fluorescence (a DNA sequencer-adapted method)^{10,11} to hydrophilic interaction liquid chromatography (HILIC)¹² or porous graphitized carbon (PGC)^{13,14} chromatography interfaced with electrospray ionization-based mass spectrometers or as a means of prefractionation prior to analysis by MALDI-MS¹⁵—for which glycans are generally permethylated prior to analysis.^{9,16}

Nearly all approaches employed in P/S glycomics focus on the analysis of intact glycans-most commonly N-linked glycans (generally to the exclusion of O-linked glycans and glycolipids). Quite commonly, accounts of such studies that are focused on cancer conclude by taking a wide-angle view of all intact glycans that were altered in cancer relative to a healthy or benign disease state and reporting unique glycan features such as core fucosylation, bisecting N-acetylglucosamine (GlcNAc), and α2-6 sialylation that were found increased or decreased in cancer.⁶ Often these features are then directly connected to the activity of specific glycosyltransferases.¹⁷ In 2013, Borges et al.¹⁸ developed a molecularly bottom-up approach to serum glycomics, which, following permethylation of an unfractionated P/S sample, employs the principles of glycan linkage analysis to break down all P/S glycans into monosaccharides in a way that maintains information about which hydroxyl groups of each monosaccharide were connected to other carbohydrate residues in the original glycan polymer¹⁸⁻²⁰ (FIGS. 1-2). This mode of P/S sample preparation results in a collection of roughly two dozen partially methylated alditol acetates (PMAAs), each of which represents a unique glycan “node” from the original glycan polymers and can readily be quantified by GC-MS. Uniquely, several PMAAs such as those arising from 2,6-linked mannose, 4,6-linked GlcNAc, and 3,4,6-linked mannose correspond to unique glycan features such as β1-6 branching, core fucosylation, and bisecting GlcNAc, respectively, and capture these unique features as single analytical signals rather than allowing the signal from that feature to be spread across all intact glycans that bear the unique feature (FIG. 1). Similarly, many of the PMAAs serve as excellent surrogates for the activities of the glycosyltransferases (GTs) that produced them—as only one or two known human GTs are capable of producing that particular glycan monosaccharide linkage pattern.¹⁸ In addition, this unique approach to P/S glycomics simultaneously captures information from all major classes of P/S glycans, including N-, O-, and lipid-linked glycans.¹⁸ The specificity of this approach with regard to producing only those chromatographic peaks for glycan nodes known to be present on a particular pre-isolated glycoprotein is illustrated within FIG. 3 of the paper in which this approach was originally described.¹⁸

### Bladder Cancer

Urothelial cell carcinoma (UCC) or bladder cancer is one of the top ten causes of cancer deaths annually [1]. From a clinical perspective, there are two major forms of this cancer: 1) non-muscle-invasive bladder cancer (NMIBC; stages pTa/pT1/pTis) and 2) muscle-invasive bladder cancer (MIBC; stages pT2+). Early detection of bladder cancer is very important; patients with non-muscle-invasive tumors have a much higher 5-year survival rateÐ88% for NMIBC patients relative to 41% for MIBC patients [2]. Yet despite the stage at which it is diagnosed, high recurrence rate is one of the essential characteristics of this cancer [3]. Therefore, even if diagnosed at early stages and treated, former bladder cancer patients need to be monitored frequently. Currently, common methods for detecting bladder cancer and monitoring for its recurrence include: cystoscopy (which is invasive and expensive [4]), urine cytology (which has low sensitivity for low-grade bladder cancer [5]), and computed tomography (CT) screening (which may not detect small tumors [6]). Accordingly, there has been a wide search for new biomarkers that are noninvasive, cost effective, and can outperform cytology [7-10].

At present, there are no clinically employed serum-based markers for monitoring patients after their treatment. Targeted glycomics, particularly when combined with other well-defined markers and risk stratification models, represents a promising source for a new generation of bladder cancer markers [11]. Some evidence toward this end based on the detection of the Sialyl Lewis^{a} antigen [12, 13] and analysis of intact N-glycans [14, 15] in blood plasma/serum (P/S) from bladder cancer patients has been obtained. Aberrant glycosylation is a universal feature of cancer [16] where it appears to enable the ability of tumor cells to avoid innate immune detection [17]. The changes in structure and abundance of glycans are often caused by dysregulated glycosyltransferase (GT) activity [16]. Thus conceptually, a targeted glycan analysis technique that could provide one-to-one surrogate data for abnormal GT activity using routinely available clinical samples and that relied upon existing clinical technology could be quite valuable.

In 2013, one of the present inventors developed a molecularly bottom-up approach called glycan node analysis that, unlike other approaches used in P/S glycomics, focuses on the analysis of monosaccharide and linkage-specific glycan “nodes” instead of intact glycans [18-21]. It does this by employing the principles and processing chemistry of glycan methylation analysis (i.e., linkage analysis; FIG. 2) to unfractionated P/S. This pools together each unique monosaccharide-and-linkage-specific glycan feature or glycan “node” from across all the normal and aberrant glycan structures in a given sample, providing a more direct surrogate measurement of GT activity than any single intact glycan. Moreover, many of these glycan nodes correspond directly and quantitatively to interesting glycan features such as “core fucosylation”, “bisecting GlcNAc”, and “β1-6 branching”—all captured as single GC-MS chromatographic peaks (FIG. 1).

### Lung Cancer

Lung cancer accounts for approximately 25% of all U.S. cancer deaths, making it the leading cause of U.S. cancer deaths.¹ More than half of lung cancer patients are diagnosed at an advanced stage: about 33% and 40% of lung cancer patients are diagnosed at stage IIIB and IV, respectively,² primarily due to a lack of early stage symptoms. The five-year survival rate of stage IV patients is only ˜5%.1 Conversely, if lung cancer can be detected before it escapes the lungs, five-year survival rates usually exceed 50%.¹ Therefore, to improve the outcomes of lung cancer patients, a major clinical priority is to detect lung cancer early. Recently, the National Lung Screening Trial (NLST) applied low dose chest computed tomography (LDCT) in older, high-risk individuals and achieved 20% reduction in lung cancer mortality. Yet the positive screening rate in this study was 24.2%, of which 96.4% were false-positive results.³ The high false-positive rate may lead to additional clinical tests, emotional distress, and unnecessary treatments, as well as unnecessary time and costs spent. Thus, a reliable and highly specific noninvasive blood test could help to reduce the false-positive and overdiagnosis rate of CT scans.

Biomarkers from easily accessible biofluids, such as blood plasma or serum (P/S), could potentially be used as a noninvasive and cost-effective way to improve lung cancer diagnosis and screening. Numerous P/S biomarkers for lung cancer have been extensively studied, including proteins (such as cytokeratin 19 fragments^{4,5} and carcinoembryonic antigen^{6,7}), miRNAs (such as miR-348 and miR-1829,10), methyl-DNA (such as P1611 and BRMS112), and circulating tumor cells.¹³ However, biomarkers with improved sensitivity and specificity are still needed.

Aberrant glycosylation is a well-established hallmark of cancer and seems to facilitate the metastasis of various tumor cells.¹⁴ Thus, blood P/S glycomics represents a promising source for a new generation of cancer biomarkers. At present, almost all P/S glycomics studies focus on the analysis of intact glycans—primarily N-linked glycans, with O-linked and lipid-linked glycans usually excluded. Generally, a great many intact glycan structures need to be investigated in order to fully capture and quantify the cancer-specific behavior of one unique glycan feature, such as core fucosylation, α2-6 sialylation, or β1-4 branching.¹⁵ Glycan node analysis is a molecularly bottom-up approach to P/S glycomics developed by Borges et al. in 2013 that focuses on monosaccharides and linkage specific glycan “nodes” rather than the intact glycan structures.¹⁶⁻²⁰ This approach captures all P/S glycans including N-, O-, and lipid-linked glycans and breaks them down into monosaccharides that maintain their original linkage information. In short, the method involves the application of glycan linkage (methylation) analysis to whole biofluids. Uniquely in this approach, linkage-related glycan features are captured and quantified as single analytical signals, rather than being spread across numerous intact glycans that bear the specific feature. For example, 6-linked galactose and 2,6-linked mannose, corresponding to α2-6 sialylation and β1-6 branching, respectively, are both captured as single chromatographic peak areas (FIG. 1). In addition, numerous glycan nodes serve as direct surrogates for the activities of specific glycosyltransferases (GTs)-enzymes that facilitate the construction of glycans.

Interestingly, there are several important gender differences in lung cancer, including the facts that (1) after adjusting for the number of cigarettes smoked, women have a 3-fold greater risk of lung cancer than men,²¹⁻²⁴ (2) never-smoker women are at significantly greater risk for lung cancer than men,²⁵ and (3) women tend to have better survival rates than men.^{26,27}

Citation of any reference in this section is not to be construed as an admission that such reference is prior art to the present disclosure.

### SUMMARY

The present disclosure provides a method of detecting altered glycan nodes in a sample from a patient having or being treated for cancer, suspected of having cancer or at risk to having cancer. The method comprises (a.) obtaining a plasma sample from the patient, wherein the plasma sample comprises glycans; (b.) permethylating the sample comprising glycans; (c.) hydrolyzing the product from step (b); (d.) reducing the product from step c; (e.) acetylating the product from step (d); (f.) partially purifying the product from step (e); and (g.) analyzing the product of step (f) using a substance identifying technique to detect altered glycan nodes in the plasma sample.

The present disclosure also provides a method of detecting terminal fucosylation, α2-6 sialylation, β1-4 branching, β1-6 branching, and outer-arm fucosylation in a sample from a patient having or being treated for cancer, suspected of having cancer or at risk to having cancer. The method comprises (a.) obtaining a sample from the patient, wherein the sample comprises glycans; (b.) permethylating the sample comprising glycans; (c.) hydrolyzing the product from step (b); (d.) reducing the product from step (c); (e.) acetylating the product from step (d); (f.) purifying the product from step (e); (g.) analyzing the product of step (f) using a substance identifying technique to terminal fucosylation, α2-6 sialylation, β1-4 branching, β1-6 branching, and outer-arm fucosylation in the sample.

The present disclosure also provides a method of detecting α2-6 sialylation, β1-4 branching, β1-6 branching, and outer-arm fucosylation in a sample from a patient having or being treated for bladder cancer, suspected of having bladder cancer or at risk to having bladder cancer. The method comprises: (a.) obtaining a sample from the patient, wherein the sample comprises glycans; (b.) permethylating the sample comprising glycans; (c.) hydrolyzing the product from step (b); (d.) reducing the product from step (c); (e.) acetylating the product from step (d); (f.) purifying the product from step (e); (g.) analyzing the product of step (f) using a substance identifying technique to terminal fucosylation, α2-6 sialylation, β1-4 branching, β1-6 branching, and outer-arm fucosylation in the sample.

### BRIEF DESCRIPTION OF FIGURES

FIG. 1 shows a conceptual overview of the glycan “node” analysis concept-which essentially consists of applying glycan linkage (methylation) analysis to whole biofluids. Intact normal and abnormal glycans including O-glycans, N-glycans and glycolipids, are processed and transformed into partially methylated alditol acetates (PMAAs, FIG. 2), each of which corresponds to a particular monosaccharide-and-linkage-specific glycan “node” in the original polymer. As illustrated, analytically pooling together the glycan nodes from amongst all the aberrant intact glycan structures provides a more direct surrogate measurement of abnormal glycosyltransferase activity than any individual intact glycan, while simultaneously converting unique glycan features such as “core fucosylation”, “α2-6 sialylation”, “bisecting GlcNAc”, and “β-6 branching” into single analytical signals. Actual extracted ion chromatograms from 9-μL blood plasma samples are shown. Numbers adjacent to monosaccharide residues in glycan structures indicate the position at which the higher residue is linked to the lower residue. Figure adapted with permission from Borges C R et al. Anal. Chem. 2013, 85(5):2927-2936.

FIG. 2 shows a molecular overview of the glycan “node” analysis procedure. For glycans from blood plasma and other biofluids, O-linked glycans are released during permethylation, while N-linked glycans and glycolipids are released during acid hydrolysis. The unique pattern of methylation and acetylation in the final partially methylated alditol acetates (PMAAs) corresponds to the unique “glycan node” in the original glycan polymer and provides the molecular basis for separation and quantification by GC-MS. Figure adapted with permission from Borges C R et al. Anal. Chem. 2013, 85(5):2927-2936.

FIG. 3 provides a summary of the case-controlled sample sets used in Example 1.

FIG. 4A-FIG. 4B provide tables of statistically significant differences between cohorts within the large lung cancer study of Example 1.

FIG. 5A-FIG. 5O show univariate distributions and associated ROC curves for the top five-performing glycan nodes in the large lung cancer set. Letters above the data points in panels a-e indicate statistically significant differences between the six groups shown: any overlap in lettering between groups indicates a lack of significant difference between the groups (Kruskal-Wallis with Dunn's post hoc test). ROC curves for lung cancer cases (separated by stage) and controls vs. certifiably healthy patients are shown in panels f-j; stage I-IV cancer patients vs. controls are shown in panels k-o. ROC curve AUCs are provided in parenthesis next to the specified stages. “NS” next to ROC curve AUCs indicates that the ROC curve does not show a statistically significant difference between the two groups being compared. Glycan node symbol definitions are the same as in FIG. 1.

FIG. 6A-6E show ROC curves depicting the stage-dependent performance of the top five-performing glycan nodes in distinguishing different types of cancer from controls or healthy individuals. Adjacent to panels b-e are ROC curves from the large lung cancer study for comparison (FIG. 6F-I). Clear stage-dependence is evident, regardless of the type of cancer involved. A comparison of each ROC curve at each stage in the large lung cancer study to the parallel ROC curve in a different type of cancer or different lung cancer sample set revealed no significant differences between ROC curves (DeLong's test; see Table S5 accompanying Ferdosi S. et al., Journal of Proteome Research 2018, 17(1):543-558.). A superscript “NE” (panels d and h) indicates that these data sets were normalized to the sum of endogenous hexoses or HexNAcs because heavy labeled internal standards were not added during analysis of the prostate cancer sample set.

FIG. 7A-FIG. 7E show univariate distributions of the top five-performing glycan nodes within the control group of the large lung cancer set, subdivided on the basis of smoking status. Letters above the data points indicate statistically significant differences between the three groups shown: any overlap in lettering between groups indicates a lack of significant difference between the groups (Kruskal-Wallis; Bonferroni-corrected p-values <0.0167 for within-group pairwise comparisons were considered statistically significant).

FIG. 8A-FIG. 8D show Multivariate logistic regression models for stage I-IV lung cancer patients from the large lung cancer data set. Fully validated multivariate combinations of glycan nodes did not produce significantly better ROC curves in stage IV, III, II, or I lung cancer patients (FIG. 8A-D, respectively) compared to the best performing individual glycan node in the control specimens (DeLong test). Three separate curves are shown on each plot, corresponding to predicted probabilities derived from a multivariate logistic regression model 1) re-fitted at each iteration of cross-validation (referred to as “CV Probabilities (full)”), 2) fitted once on the complete dataset, fixing the predictors, but allowing parameter estimates to change at each iteration of cross-validation (referred to as “CV Probabilities (semi)”), and 3) fitted once on the complete dataset and taking the model-derived probability without use of cross-validation (referred to as “Fitted Probabilities”).

FIG. 9A-FIG. 9F show Univariate distributions of fucosylation-related glycan nodes, α2-6 sialylation, β1-4 branching and β1-6 branching in stage 0 through stage IV liver fibrosis. No statistically significant differences were observed for any pairwise comparisons within a single glycan node (Kruskal-Wallis).

FIG. 10A-FIG. 10D show a large lung cancer data set progression (i.e., progression-free survival; FIG. 10A, 10C) and survival (all-cause mortality; FIG. 10B,10D) curves for the top α2-6 sialylation quartile compared to all other quartiles combined. Panels a-b combine data from all stages; panels c-d present data from stage III only-illustrating that curve separation based on α2-6 sialylation is not simply driven by stage. Dotted lines represent 95% confidence intervals, colored according to their respective curves. Within each plot, progression curves were significantly different from one another (log-rank Mantel-Cox test; p<0.01) as were the survival curves (log-rank Mantel-Cox test; p<0.05). For the progression data (all stages; panel a), the median duration of follow-up for those that progressed, until progression, was 6.9 months (17.1 months median total follow-up time); for those that did not progress, the median duration of follow-up was 22.7 months. Results from Cox proportional hazards models are described in the Results section.

FIGS. 11A-11H show distributions and ROC curves for the most highly elevated glycan node markers in former & current UCC patients relative to healthy controls when data were normalized to heavy glucose or heavy GlcNAc. Patient distributions are shown in FIGS. 11A-11D. The Kruskal-Wallis test was performed followed by Dunn's post hoc test. The letters at the top of the data points show statistically significant differences between the patient groups; groups with same letter do not have a significant difference. (FIGS. 11E-11H). ROC curves for the different sub-cohorts of UCC patients vs. healthy individuals. Areas under the ROC curves are provided in parenthesis next to the stated patient groups. As explained in Example 2, despite the promising AUCs and shapes of some of these ROC curves, these data do not indicate that plasma/serum glycan nodes will potentially serve as clinically useful diagnostic markers of UCC.

FIG. 12A-FIG. 12H show distributions and ROC curves for the most highly elevated glycan node markers in former & current UCC patients relative to healthy controls when data were normalized to sum of endogenous Hexoses or HexNAcs. Patient distributions are shown in (a-d). The Kruskal-Wallis test was performed followed by Dunn's post hoc test. The letters at the top of the data points show statistically significant differences between the patient groups; groups with a common letter do not have a significant difference. (e-h) ROC curves for different groups of bladder cancer patients vs. certifiably healthy individuals. Area under the ROC curves are provided in parenthesis next to the stated patient groups. “NS” next to the area under the ROC curves shows that there is no significant difference between the two groups that are being compared. These data do not indicate that plasma/serum glycan nodes will potentially serve as clinically useful diagnostic markers of UCC.

FIG. 13A-FIG. 13D show correlation between age and the most highly elevated glycan node markers in former & current UCC patients relative to healthy controls when data were normalized to heavy glucose or heavy GlcNAc. Pearson correlation was used to evaluate this correlation. The common age range between all cohorts was 45-67. “NS” next to the r-value indicates that the Pearson correlation was not statistically significant. Distribution of the healthy controls is demonstrated by red dots. Distribution of the different sub-cohorts of UCC patients is demonstrated by black triangles.

FIG. 14A-FIG. 14B show bladder cancer recurrence curves for: (a) The top α2-6 sialylation quartile compared to all other quartiles combined. (b) The to β1-6 branching quartile compared to all other quartiles combined. In both panels, the recurrence curves within each plot were significantly different (log-rank Mantel-Cox test; p<0.05). The median duration of follow-up for those that relapsed, until relapse was 6 months, and for those that did not relapse was 12 months (The median total follow-up time was 11.75 months). The results of Cox proportional hazards models are reported in the Results section.

FIG. 15A-FIG. 15B show correlation of CRP and glycan nodes. Log of CRP concentration vs. (a) α2-6 sialylation; r=0.34 and (b) β1-6 branching; r=0.38 are plotted. Both correlations are statistically significant (Pearson correlation; p<0.001).

FIG. 16A-FIG. 16D show distribution of the most highly elevated glycan node markers in former & current UCC patients relative to healthy controls with the MIBC group separated by patient stage. Data from Example 1 are displayed side-by-side for qualitative comparison. “SM Controls” indicates smoking status matched to the lung cancer patients on the basis of “current”, “former”, or “never-” smoker status. Letters at the top of each cohort show statistically significant differences between the patient groups; groups with a common letter do not have a significant difference.

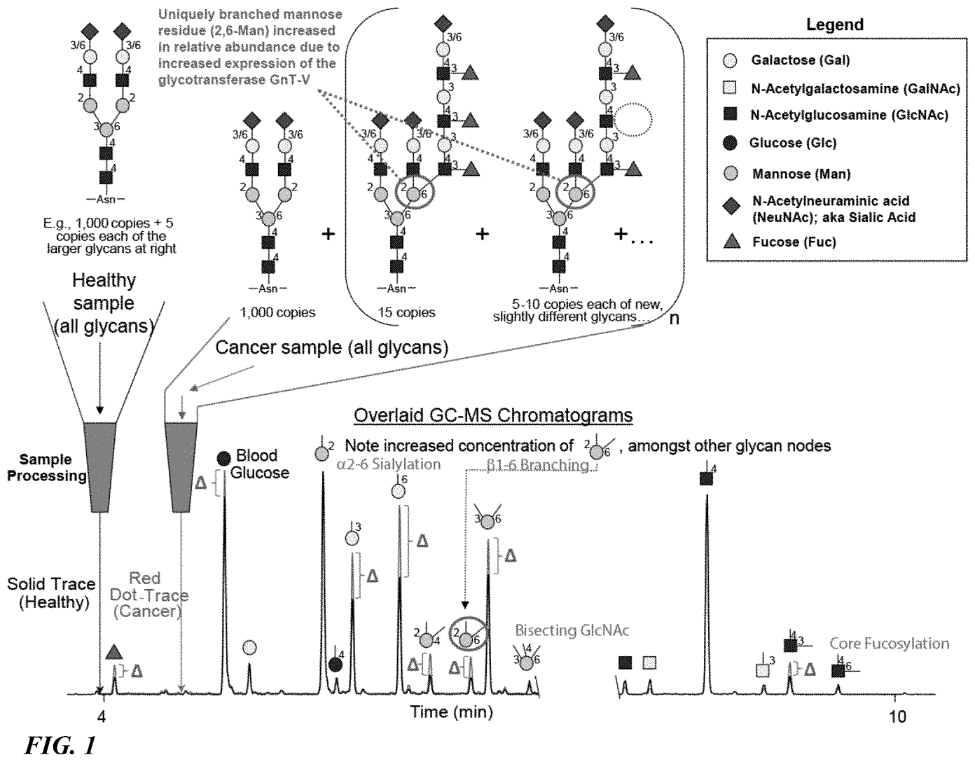

### DETAILED DESCRIPTION

Certain illustrative embodiments include the following:

1. A method of detecting altered glycan nodes in a sample from a patient having or being treated for cancer, suspected of having cancer or at risk to having cancer, the method comprising:
a. obtaining a sample from the patient, wherein the sample comprises glycans;
b. permethylating the sample comprising glycans;
c. hydrolyzing the product from step (b);
d. reducing the product from step (c);
e. acetylating the product from step (d);
f. partially purifying the product from step (e);
g. analyzing the product of step (f) using a substance identifying technique to detect altered glycan nodes in the sample.
   2. The method of the above 1, wherein the altered glycan nodes are selected from the group consisting of terminal fucose; 6-linked galactose; 2,4-linked mannose; 2,6-linked mannose; and 3,4-linked Nacetylglucosamine.
   3. The method of the above 1, wherein the cancer is selected from the group consisting of lung cancer, prostate cancer, ovarian cancer and pancreatic cancer.
   4. The method of the above 1, wherein the sample is plasma.
   5. The method of the above 1, wherein step (b) includes an initial substep of mixing the sample comprising glycans with a labeled chemical substance.
   6. The method of the above 4, wherein the labeled chemical substance is heavy-labeled D-glucose, N-acetyl-D-[UL-¹³C₆]glucosamine, or combinations thereof.
   7. The method of the above 1, wherein step (b) comprises liquid/liquid extraction.
   8. The method of the above 1, wherein step (c) uses trifluoroacetic acid.
   9. The method of the above 1, wherein step (d) uses a reducing agent selected from the group consisting of NaBH₄, NaBD₄ and a combination thereof.
   10. The method of the above 1, wherein step (e) uses acetic anhydride.
   11. The method of the above 1, wherein step (f) comprises liquid/liquid extraction.
   12. The method of the above 1, wherein the substance identifying technique of step (g) is GC-MS.
   13. A method of detecting terminal fucosylation, α2-6 sialylation, β1-4 branching, β1-6 branching, and outer-arm fucosylation in a sample from a patient having or being treated for cancer, suspected of having cancer or at risk to having cancer, the method comprising:
a. obtaining a sample from the patient, wherein the sample comprises glycans;
b. permethylating the sample comprising glycans;
c. hydrolyzing the product from step (b);
d. reducing the product from step (c);
e. acetylating the product from step (d);
f. purifying the product from step (e);
g. analyzing the product of step (f) using a substance identifying technique to terminal fucosylation, α2-6 sialylation, β1-4 branching, β1-6 branching, and outer-arm fucosylation in the sample.
   14. The method of the above 13, wherein the cancer is selected from the group consisting of lung cancer, prostate cancer, ovarian cancer and pancreatic cancer.
   15. The method of the above 13, wherein the sample is plasma.
   16. The method of the above 13, wherein step (b) includes an initial substep of mixing the sample comprising glycans with a labeled chemical substance.
   17. The method of the above 16, wherein the labeled chemical substance is heavy-labeled D-glucose, N-acetyl-D-[UL-¹³C₆]glucosamine, or combinations thereof.
   18. The method of the above 13, wherein step (b) comprises liquid/liquid extraction.
   19. The method of the above 13, wherein step (c) uses trifluoroacetic acid.
   20. The method of the above 13, wherein step (d) uses a reducing agent selected from the group consisting of NaBH₄, NaBD₄ and a combination thereof.
   21. The method of the above 13, wherein step (e) uses acetic anhydride.
   22. The method of the above 13, wherein step (f) comprises liquid/liquid extraction.
   23. The method of the above 13, wherein the substance identifying technique of step (g) is GC-MS.
   24. A method of detecting α2-6 sialylation, β1-4 branching, β1-6 branching, and outer-arm fucosylation in a sample from a patient having or being treated for bladder cancer, suspected of having bladder cancer or at risk to having bladder cancer, the method comprising:
a. obtaining a sample from the patient, wherein the sample comprises glycans;
b. permethylating the plasma sample comprising glycans;
c. hydrolyzing the product from step (b);
d. reducing the product from step (c);
e. acetylating the product from step (d);
f. purifying the product from step (e);
g. analyzing the product of step (f) using a substance identifying technique to terminal fucosylation, α2-6 sialylation, β1-4 branching, β1-6 branching, and outer-arm fucosylation in the sample.
   25. The method of the above 24, wherein the sample is plasma.
   26. The method of the above 24, wherein step (b) includes an initial substep of mixing the sample comprising glycans with a labeled chemical substance.
   27. The method of the above 26, wherein the labeled chemical substance is heavy-labeled D-glucose, N-acetyl-D-[UL-¹³C₆]glucosamine, or combinations thereof.
   28. The method of the above 24, wherein step (b) comprises liquid/liquid extraction.
   29. The method of the above 24, wherein step (c) uses trifluoroacetic acid.
   30. The method of the above 24, wherein step (d) uses a reducing agent selected from the group consisting of NaBH₄, NaBD₄ and a combination thereof.
   31. The method of the above 24, wherein step (e) uses acetic anhydride.
   32. The method of the above 24, wherein step (f) comprises liquid/liquid extraction.
   33. The method of the above 24, wherein the substance identifying technique of step (g) is GC-MS.
   34. A method of detecting altered glycan nodes in a sample from a patient having or being treated for cancer, suspected of having cancer or at risk to having cancer, the method comprising:
a. obtaining a sample from the patient, wherein the sample comprises glycans;
b. permethylating the sample comprising glycans to form a product;
c. hydrolyzing the product to form a second product;
d. reducing the second product to form a third product;
e. acetylating the third product to form a fourth product;
f. partially purifying the fourth product to form a fifth product;
g. analyzing the fifth product using a substance identifying technique to detect altered glycan nodes in the sample.
   35. A method of detecting terminal fucosylation, α2-6 sialylation, β1-4 branching, β1-6 branching, and outer-arm fucosylation in a sample from a patient having or being treated for cancer, suspected of having cancer or at risk to having cancer, the method comprising:
a. obtaining a sample from the patient, wherein the sample comprises glycans;
b. permethylating the sample comprising glycans to form a product;
c. hydrolyzing the product to form a second product;
d. reducing the second product to form a third product;
e. acetylating the third product to form a fourth product;
f. purifying the fourth product to form a fifth product;
g. analyzing the fifth product using a substance identifying technique to terminal fucosylation, α2-6 sialylation, β1-4 branching, β1-6 branching, and outer-arm fucosylation in the sample.
   36. A method of detecting α2-6 sialylation, β1-4 branching, β1-6 branching, and outer-arm fucosylation in a sample from a patient having or being treated for bladder cancer, suspected of having bladder cancer or at risk to having bladder cancer, the method comprising:
a. obtaining a sample from the patient, wherein the sample comprises glycans;
b. permethylating the plasma sample comprising glycans to form a product;
c. hydrolyzing the product to form a second product;
d. reducing the second product to form a third product;
e. acetylating the third product to form a fourth product;
f. purifying the fourth product to form a fifth product;
g. analyzing the fifth product using a substance identifying technique to terminal fucosylation, α2-6 sialylation, β1-4 branching, β1-6 branching, and outer-arm fucosylation in the sample.

### Definitions

Unless defined otherwise, all technical and scientific terms used herein have the same meaning as those commonly understood by one of ordinary skill in the art to which this invention belongs. Although methods and materials similar or equivalent to those described herein can be used in the practice or testing of the present disclosure, suitable methods and materials are described below. The materials, methods and examples are illustrative only, and are not intended to be limiting. All publications, patents and other documents mentioned herein are incorporated by reference in their entirety.

Throughout this specification, the word “comprise” or variations such as “comprises” or “comprising” will be understood to imply the inclusion of a stated integer or groups of integers but not the exclusion of any other integer or group of integers.

The term “a” or “an” may mean more than one of an item.

The terms “and” and “or” may refer to either the conjunctive or disjunctive and mean “and/or”.

The term “about” means within plus or minus 10% of a stated value. For example, “about 100” would refer to any number between 90 and 110.

The term “patient” means an animal, preferably a mammal, and most preferably, a mouse, rat, other rodent, dog, cat, swine, cattle, sheep, horse, or primate, and even more preferably a human.

### Methods of Use

The present disclosure provides a method of detecting altered glycan nodes in a sample from a patient having or being treated for cancer, suspected of having cancer or at risk to having cancer. The method comprises (a.) obtaining a plasma sample from the patient, wherein the plasma sample comprises glycans; (b.) permethylating the sample comprising glycans; (c.) hydrolyzing the product from step (b); (d.) reducing the product from step c; (e.) acetylating the product from step (d); (f.) partially purifying the product from step (e); and (g.) analyzing the product of step (f) using a substance identifying technique to detect altered glycan nodes in the plasma sample.

In some embodiments, the altered glycan nodes are selected from the group consisting of terminal fucose; xylose (any linkage); terminal galactose; 2-linked mannose; 4-linked galactose; 4-linked mannose; 4-linked glucose; 3-linked mannose; 2-linked galactose; 3-linked galactose; 6-linked glucose; 6-linked mannose; 6-linked galactose; 3,4-linked galactose; 2,3-linked galactose; 2,4-linked mannose; 4,6-linked glucose; 2,6-linked mannose; 3,6-linked mannose; 3,4,6-linked mannose; terminal N-acetylglucosamine (GlcNAc); terminal N-acetylgalactosamine (GalNAc); 4-linked GlcNAc; 3-linked GlcNAc; 3-linked GalNAc; 6-linked GlcNAc; 3,4-linked GlcNAc. 4-linked GalNAc; 6-linked GalNAc; 4,6-linked GlcNAc; and 3,6-linked GalNAc.

In the methods described herein, the cancer is any type of cancer. In some embodiments, the cancer is selected from the group consisting of lung cancer, prostate cancer, ovarian cancer, pancreatic cancer and bladder cancer.

The samples used in the methods described herein may be obtained from a patient. The sample may be blood plasma, serum, sputum, seminal fluid, urine, saliva, skin, prostatic fluid, tissue, other biofluid optionally derived from tissue ex vivo, microvesicles/exosomes from both serum and urine or combinations thereof. In some embodiments, the sample is derived from a diseased organ, tissue or secretion therefrom. Samples derived from a diseased organ or tissue include, but not limited to, sputum, prostatic fluid or semen, lung tissue, breast tissue, liver tissue, colon tissue and prostate tissue. In some embodiments, the sample is plasma.

In some embodiments, step (b) includes an initial substep of mixing the sample comprising glycans with a labeled chemical substance. In one aspect of this embodiment, the labeled chemical substance is heavy-labeled D-glucose. In other aspects, the labeled chemical substance is N-acetyl-D-[UL-¹³C₆]glucosamine. In other aspects, the labeled chemical substance is a combination of heavy-labeled D-glucose and N-acetyl-D-[UL-¹³C₆]glucosamine.

In some embodiments, step (b) comprises liquid/liquid extraction. Liquid/liquid extraction, in some embodiments, comprises adding a solution of NaCl followed by a halogenated solvent. The halogenated solvent can be methylene chloride or chloroform. Adding the solution of NaCl prior to the halogenated solvent reduces the number of liquid/liquid extraction steps needed to partially purify the permethylated glycans.

In step (c), the permethylated glycans are hydrolyzed. The permethylated glycans can be hydrolyzed according to any method known in the art. In some embodiments, step (c) uses acid. In some embodiments, step (c) uses trifluoroacetic acid.

In step (d), the product of step (c) is reduced to partially permethylated alditols. The reducing agent used in this step can be any known in the art. In some embodiments, step (d) uses a reducing agent selected from the group consisting of NaBH₄, NaBD₄ and a combination thereof.

In step (e), the product of step (d) is acetylated to form partially methylated alditol acetates. The aceylation step can be performed using any known acetylated reagent. In some embodiments, step (e) uses acetic anhydride.

In step (f), the product of step (e) is partially purified. As used herein, the term “partially purifying” refers to methods of at least partially removing the product from a mixture of other compounds. In some embodiments, partially purifying the product of step (e) comprises liquid/liquid extraction.

The substance identifying technique used in the methods described herein is selected from gas chromatography-mass spectrometry (GC-MS), liquid chromatography-mass spectrometry (LC-MS), gas Chromatography coupled to tandem mass spectrometry (GC-MS/MS) and liquid chromatography with tandem mass spectrometry (LC-MS/MS). In some embodiments, the substance identifying technique of step (g) is GC-MS.

The present disclosure also provides a method of detecting terminal fucosylation, α2-6 sialylation, β1-4 branching, β1-6 branching, or outer-arm fucosylation in a sample from a patient having or being treated for cancer, suspected of having cancer or at risk to having cancer. The method comprises (a.) obtaining a sample from the patient, wherein the sample comprises glycans; (b.) permethylating the sample comprising glycans; (c.) hydrolyzing the product from step (b); (d.) reducing the product from step (c); (e.) acetylating the product from step (d); (f.) purifying the product from step (e); (g.) analyzing the product of step (f) using a substance identifying technique to terminal fucosylation, α2-6 sialylation, β1-4 branching, β1-6 branching, and outer-arm fucosylation in the sample.

In some embodiments, step (b) includes an initial substep of mixing the sample comprising glycans with a labeled chemical substance. In one aspect of this embodiment, the labeled chemical substance is heavy-labeled D-glucose. In other aspects, the labeled chemical substance is N-acetyl-D-[UL-¹³C₆]glucosamine. In other aspects, the labeled chemical substance is a combination of heavy-labeled D-glucose and N-acetyl-D-[UL-¹³C₆]glucosamine.

In some embodiments, step (b) comprises liquid/liquid extraction. Liquid/liquid extraction, in some embodiments, comprises adding a solution of NaCl followed by a halogenated solvent. The halogenated solvent can be methylene chloride or chloroform. Adding the solution of NaCl prior to the halogenated solvent reduces the number of liquid/liquid extraction steps needed to partially purify the permethylated glycans.

In step (c), the permethylated glycans are hydrolyzed. The permethylated glycans can be hydrolyzed according to any method known in the art. In some embodiments, step (c) uses acid. In some embodiments, step (c) uses trifluoroacetic acid.

In step (d), the product of step (c) is reduced to partially permethylated alditols. The reducing agent used in this step can be any known in the art. In some embodiments, step (d) uses a reducing agent selected from the group consisting of NaBH₄, NaBD₄ and a combination thereof.

In step (e), the product of step (d) is acetylated to form partially methylated alditol acetates. The aceylation step can be performed using any known acetylated reagent. In some embodiments, step (e) uses acetic anhydride.

In step (f), the product of step (e) is partially purified. As used herein, the term “partially purifying” refers to methods of at least partially removing the product from a mixture of other compounds. In some embodiments, partially purifying the product of step (e) comprises liquid/liquid extraction.

The present disclosure also provides a method of detecting α2-6 sialylation, β1-4 branching, β1-6 branching, and outer-arm fucosylation in a sample from a patient having or being treated for bladder cancer, suspected of having bladder cancer or at risk to having bladder cancer. The method comprises: (a.) obtaining a sample from the patient, wherein the sample comprises glycans; (b.) permethylating the sample comprising glycans; (c.) hydrolyzing the product from step (b); (d.) reducing the product from step (c); (e.) acetylating the product from step (d); (f.) purifying the product from step (e); (g.) analyzing the product of step (f) using a substance identifying technique to terminal fucosylation, α2-6 sialylation, β1-4 branching, β1-6 branching, and outer-arm fucosylation in the sample.

In some embodiments, step (b) includes an initial substep of mixing the sample comprising glycans with a labeled chemical substance. In one aspect of this embodiment, the labeled chemical substance is heavy-labeled D-glucose. In other aspects, the labeled chemical substance is N-acetyl-D-[UL-¹³C₆]glucosamine. In other aspects, the labeled chemical substance is a combination of heavy-labeled D-glucose and N-acetyl-D-[UL-¹³C₆]glucosamine.

In some embodiments, step (b) comprises liquid/liquid extraction. Liquid/liquid extraction, in some embodiments, comprises adding a solution of NaCl followed by a halogenated solvent. The halogenated solvent can be methylene chloride or chloroform. Adding the solution of NaCl prior to the halogenated solvent reduces the number of liquid/liquid extraction steps needed to partially purify the permethylated glycans.

In step (c), the permethylated glycans are hydrolyzed. The permethylated glycans can be hydrolyzed according to any method known in the art. In some embodiments, step (c) uses acid. In some embodiments, step (c) uses trifluoroacetic acid.

In step (d), the product of step (c) is reduced to partially permethylated alditols. The reducing agent used in this step can be any known in the art. In some embodiments, step (d) uses a reducing agent selected from the group consisting of NaBH₄, NaBD₄ and a combination thereof.

In step (e), the product of step (d) is acetylated to form partially methylated alditol acetates. The aceylation step can be performed using any known acetylated reagent. In some embodiments, step (e) uses acetic anhydride.

In step (f), the product of step (e) is partially purified. As used herein, the term “partially purifying” refers to methods of at least partially removing the product from a mixture of other compounds. In some embodiments, partially purifying the product of step (e) comprises liquid/liquid extraction.

The methods described herein can be used in diagnostic applications to predict progression, recurrence, and survival in cancer patients.

The methods described herein can be used in conjunction with methods of treatment. In these embodiments, the method further comprises administering a treatment to the patient comprising one or more therapeutic agents for treating the cancer. The amount and nature of the therapeutic agent can be varied depending on the diagnosis or predicted progression, recurrence or survival.

In order that this invention be more fully understood, the following examples are set forth.

These examples are for the purpose of illustration only and are not to be construed as limiting the scope of the invention in any way.

### EXAMPLES

### Example 1

To date, results from pilot studies in which this methodology was applied to (mostly) advanced stages of lung¹⁸ and breast cancer²⁰ have been reported. In order to gain a representative perspective on the potential utility of this approach to detecting a variety of different types of cancer at varying stages, we have now applied it to over 950 clinical P/S samples from 7 different case control studies across all stages of cancer in which the cancer cases were compared to related benign conditions and/or healthy controls. A study of plasma samples from 428 Stage I-IV lung cancer patients, age/gender/smoking-status matched controls, and certifiably healthy living kidney donors serves as the backbone for this report—in which plasma from a single donor served as a quality control specimen in every single batch of samples—facilitating comparisons to pancreatic (rapid autopsy), ovarian (Stage III), prostate (Stage II), and a large independent lung cancer (Stage I) case-control study. Based on the behavior of P/S glycans established to date, we hypothesized that the alteration of P/S glycans observed in cancer would be independent of the tissue in which the tumor originated yet exhibit stage dependence that varied little across cancers classified on the basis of tumor origin.

### Materials

Heavy, stable-isotope-labeled D-glucose (U-¹³C₆, 99%; 1,2,3,4,5,6,6-D7, 97%-98%) was purchased from Cambridge Isotope Laboratories. N-Acetyl-D-[UL-¹³C₆]glucosamine was obtained from Omicron Biochemicals, Inc. Methanol was purchased from Honeywell Burdick and Jackson. Acetone was obtained from Avantor Performance Materials. Acetonitrile and dichloromethane were acquired from Fisher Scientific. Chloroform, sodium hydroxide beads (20-40 mesh) DMSO, iodomethane (99%, catalog no. 18507), trifluoroacetic acid (TFA), ammonium hydroxide, sodium borohydride, and acetic anhydride were obtained from Sigma-Aldrich. Pierce spin columns (0.9 mL volume) including plugs were purchased from ThermoFisher Scientific (Waltham, Mass., catalog no. 69705). GC-MS autosampler vials and Teflon-lined pierceable caps were acquired from ThermoFisher Scientific. GC consumables were purchased from Agilent; MS consumables were obtained from Waters.

### Plasma and Serum Samples

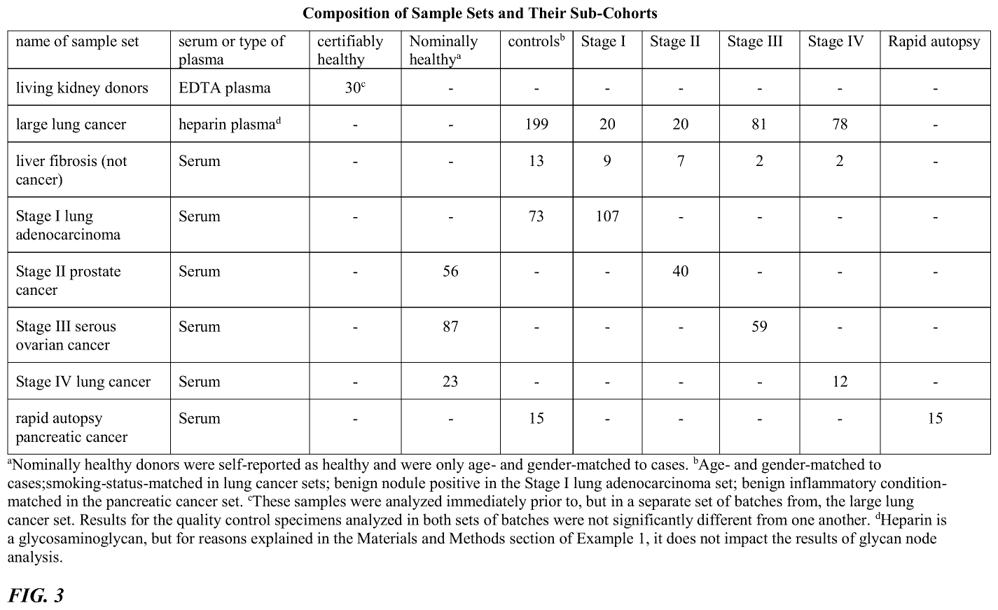

A summary of the case-control sample sets employed in this study is provided in FIG. 3. All specimens were collected in compliance with the Declaration of Helsinki principles. Once collected, they were coded and de-identified to protect patient identities.

Living Kidney Donors. EDTA plasma samples from certifiably healthy living kidney donors were enrolled in the Multidisciplinary Biobank at Mayo Clinic Arizona under a Mayo Clinic Institutional Review Board (IRB)-approved protocol. Patients eligible for enrollment were those seen at Mayo Clinic Arizona who were ≥18 years old, able to provide informed consent, and undergoing evaluation as a potential living kidney donor. Detailed inclusion and exclusion criteria for these patients are provided in the Supporting Information for Ferdosi S. et al., Journal of Proteome Research 2018, 17(1):543-558. None of these patients smoked at the time of health screening and blood collection; 27% were former smokers, and 73% never smoked. Specimens were collected over a 2-year period from December 2013 to December 2015. Standard operating protocols and blood collections were performed as previously described.²¹ All specimens were stored at −80° C. prior to shipment to Arizona State University.

Large Lung Cancer Set. Sodium heparin plasma samples for the large lung cancer study were collected at the University of Texas MD Anderson Cancer Center under the supervision of Dr. Xifeng Wu. Heparin is a glycosaminoglycan itself but the vast majority of its monomer units are carboxylated, sulfated, or both. As previously described,¹⁸ sulfated and carboxylated glycan monomers cannot be directly detected by the analytical methodology employed in this study. The PMAA from 4-linked GlcNAc could theoretically be produced by the heparin anticoagulant, but empirically, was found in matched collection studies (described in the Results of Example 1) that 4-linked GlcNAc from heparin plasma is not significantly different from EDTA plasma or serum. Specimens for lung cancer cases and controls from the University of Texas MD Anderson Cancer Center included in this example are part of an ongoing large lung cancer study that has been recruiting since 1995. This study has received approval from the University of Texas MD Anderson Cancer Center and Kelsey-Seybold institutional review boards. Venous blood was drawn from newly diagnosed and histologically confirmed lung cancer patients (prior to therapy) and age-, gender-, and ethnicity-matched controls at the MD Anderson Cancer Center hospital and the nearby Kelsey-Seybold Clinic, respectively. All blood was drawn and processed under the same SOP. Patients were not necessarily in a fasted state. Blood was centrifuged then aliquoted and placed into a liquid nitrogen tank. After collection, samples were coded and de-identified prior to shipment to Arizona State University for analysis. A more-detailed profile of the clinical characteristics of the patients in this large lung cancer study is provided in Table S1 accompanying Ferdosi S. et al., Journal of Proteome Research 2018, 17(1):543-558.

Liver Fibrosis (Non-Cancerous). Serum samples from patients at all stages of liver fibrosis were collected at the Sunnybrook Health Sciences Centre, under the direction of Dr. Lei Fu and Dr. David E. C. Cole. This study was approved by Research Ethics Board, Sunnybrook Health Sciences Centre, Toronto. Patients were recruited between 2007 and 2011. Written informed consent was obtained from each participant. All subjects with various chronic liver diseases were considered eligible if they would have liver biopsy for the diagnosis of liver fibrosis as part of their routine care. Blood specimens were collected, and serum was separated from cells following standard clinical laboratory procedures. Serum aliquots were stored in −70° C. The specimens were coded and de-identified according to the study protocol.

Stage I Lung Adenocarcinoma. Serum samples from stage I lung adenocarcinoma patients and age-, gender-, and smoking-status-matched controls were collected under NYU IRB approval at the NYU Langone Medical Center by Dr. Harvey Pass. Arterial blood samples were collected from fasting patients undergoing surgery in the time frame from September 2006 to August 2013 to remove one or more lung nodules that were detected during a CT scan. Determination of whether nodules were benign or malignant was made following a pathological exam of the excised nodules. Serum was collected in standard glass serum tubes and allowed to sit upright for 30-60 min to allow clotting. Subsequently, tubes were centrifuged at 1200 g for 20 min at room temperature, then aliquoted and placed at −80° C. within 2-3 h of collection. No freeze-thaw cycles occurred prior to shipment to Arizona State University (Borges lab) for analysis.

Stage II Prostate Cancer. Serum samples from stage II prostate cancer patients were obtained from the Cooperative Human Tissue Network (CHTN), an NIH-sponsored biospecimen collection agency. The quality management system of the CHTN is described elsewhere.²² Age-matched control samples from nominally healthy male donors were obtained from ProMedDx (Norton, Mass.).

Stage III Serous Ovarian Cancer. Serum specimens from stage III serous ovarian cancer patients were collected at Brigham and Women's Hospital under IRB approval by Dr. Daniel Cramer. Sera were obtained at the time of presentation prior to surgery. Age, gender, and location matched control sera from women without a history of cancer (other than nonmelanoma skin cancer) were obtained from the general population under a standardized serum collection protocol. All serum samples were collected from 2001 to 2010 and were stored at −80° C. prior to analysis. These specimens have previously been described.^{23,24}

Stage IV Lung Cancer. A set of serum samples from stage IV lung cancer patients and age- and gender-matched nominally healthy control donors that was completely separate from those provided by Dr. Xifeng Wu at the University of Texas MD Anderson Cancer Center was obtained from ProMedDx.

Rapid Autopsy Pancreatic Cancer. Serum specimens from rapid autopsy patients who had recently died from pancreatic cancer were collected by Dr. Michael Hollingsworth at the University of Nebraska Medical Center under IRB approval. These samples have previously been described.²⁵ In brief, specimens were collected within 2-3 h of death. Control serum samples were from patients with benign pancreatic conditions and elevated CA19-9 levels. Samples were coded, de-identified, and kept at −80° C. prior to shipment to Arizona State University.

Additional Biospecimen Details. As described above, all blood samples were processed into P/S immediately following collection and stored at −70° C. or colder until analyzed. Following shipment in dry ice, vial headspace was vented prior to thawing to avoid CO₂-mediated sample acidification.²⁶ The molecular integrity of the sample set that showed the greatest differences between cases and controls (rapid autopsy pancreatic cancer sera) was examined using an assay based on ex vivo protein oxidation that was recently developed by the Borges group.²⁷ The prostate cancer and stage I lung adenocarcinoma sets were spot-checked as well. No samples produced evidence for concern about specimen integrity.

In this example, multiple independent sets of sample were compared to each other. Each case-control set was analyzed blind and in random order. Within each batch, across all sets, a quality control (QC) EDTA plasma sample was included consisting of a 9 uL aliquot of the same bulk plasma sample to verify the reproducibility across batches. Notably, the samples from the certifiably healthy living kidney donors were analyzed in separate batches of samples from those in the large lung cancer set. To justify direct comparison of these two sets of samples, we verified that the average values measured for each glycan node in the two sets of QC sample results were not statistically significantly different. Moreover, if the average value of the QC sample was slightly higher or lower in the large lung cancer set relative to the living kidney donor set a scaling factor based on this difference in QC samples was employed to adjust the living kidney donor data set. For each glycan node, this adjustment brought the living kidney donor data set distribution slightly closer to the control distributions observed in the large lung cancer set, meaning that it was a conservative adjustment. Furthermore, to validate the comparability of results in serum and multiple different types of plasma, the glycan “node” analysis procedure was applied to matched sets of P/S samples from 21 donors. This set consisted of four different types of plasma and a serum sample from each donor. The difference between these four types of plasma was based on the different anticoagulants, which were K₂EDTA, K₃EDTA, sodium EDTA, and 3.8% sodium citrate. In an additional study, six matched-collection aliquots of serum, K₂EDTA plasma, and heparin plasma from a single donor were analyzed and compared to each other to verify the consistency of glycan nodes between the aforementioned types of samples.

### Experimental Procedures

The global glycan methylation analysis procedure consisted of five main steps; permethylation, trifluoroacetic acid (TFA) hydrolysis, reduction of sugar aldehydes, acetylation of nascent hydroxyl groups, and final cleanup.^{18,19} Each step is described in detail in the following.

Permethylation, Nonreductive Release, and Purification of Glycans. A total of 9 μL of P/S was added into a 1.5 mL eppendorf tube followed by 1 μL of a 10 mM solution of heavy-labeled D-glucose (U-¹³C₆, 99%; 1,2,3,4,5,6,6-D7, 97%-98%), and N-acetyl-D-[UL-¹³C₆]glucosamine, which served as internal standards for relative quantification. Then, 270 μL of dimethyl sulfoxide (DMSO) was added to the biological sample and mixed to dissolve completely. Once the sample was fully dissolved, 105 μL of iodomethane was added to the mixture. This solution was then added to a plugged 1 mL spin column, which contained ˜0.7 g of sodium hydroxide beads. The NaOH beads had been preconditioned with acetonitrile and rinsed with DMSO twice before the sample was added. Then, the NaOH column was stirred occasionally for 11 min. When finished, samples were unplugged and spun for 15 s at 5000 rpm (2400 g) in a microcentrifuge to extract the glycan-containing solution. To wash off all the permethylated glycan, 300 μL of acetonitrile was added to the spin column and then centrifuged for 30 s at 10 000 rpm (9600 g). Then, samples from the first spin-through were placed in a silanized 13×100 mm glass test tube containing approximately 3.5 mL of 0.5 M NaCl solution in 0.2 M sodium phosphate buffer (pH 7.0) and mixed well. Next, the second spin-through was pooled with the rest of the sample, avoiding the white residue at the bottom of the spin column. The test tube was capped and shaken thoroughly after adding 1.2 mL of chloroform to the sample. Liquid/liquid extraction was performed three times, saving the chloroform layer. The chloroform layer was then extracted with a silanized pipet, transferred to a silanized glass test tube, and dried under nitrogen at heater-block temperature setting of 74° C.

TFA Hydrolysis. A total of 325 μL of 2 M TFA was added to each sample. Samples were then capped and heated at 121° C. for 2 h. Afterward, samples were dried down under nitrogen at 74° C.

Reduction of Sugar Aldehydes. A total of 475 μL of a freshly prepared 10 mg/mL solution of sodium borohydride in 1 M ammonium hydroxide was added to each test tube. After the sample was allowed to react for 1 h at room temperature, 63 μL of methanol was added to each sample and then dried down at 74° C. under nitrogen. A solution of 9:1 (v/v) methanol/acetic acid was then prepared, and 125 μL was added to each test tube, which was again dried under nitrogen. Before moving forward, the samples were fully dried in a vacuum desiccator for at least 15-20 min.

Acetylation of Nascent Hydroxyl Groups. A total of 18 μL of water was added to each sample and mixed well to dissolve the entire sample residue. A total of 250 μL of acetic anhydride was then added to each sample. Next, the sample was sonicated in a water bath for 2 min, followed by an incubation for 10 min at 60° C. A total of 230 μL of concentrated TFA was then added to each test tube. The capped test tube was then incubated at 60° C. for 10 min.

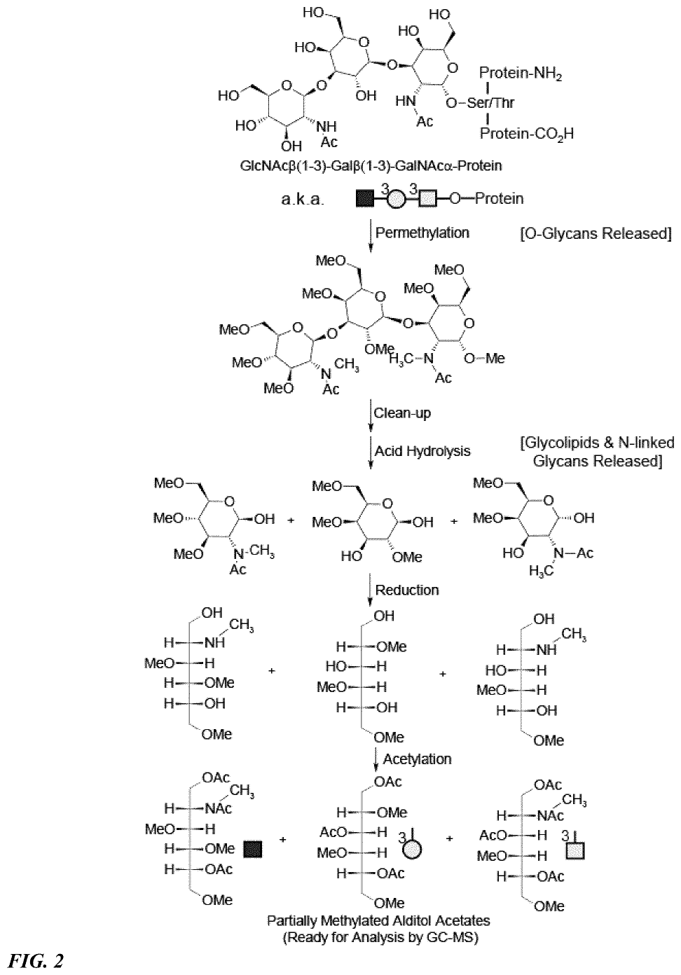

Final Cleanup. Approximately 2 mL of methylene chloride was added to each test tube and mixed well. Then, 2 mL water was added to each sample and mixed well. Liquid/liquid extraction was performed twice, saving the organic layer. Next, the organic layer was transferred with a silanized glass pipet into a silanized autosampler vial. The organic layer was then evaporated under nitrogen, reconstituted in 120 μL of acetone and capped for injection onto GC-MS. A molecular overview of the global glycan methylation analysis procedure is shown in FIG. 2.

Gas Chromatography-Mass Spectrometry. For sample analysis, an Agilent Model A7890 gas chromatograph (equipped with a CTC PAL autosampler) was used coupled to a Waters GCT (time-of-flight) mass spectrometer. A total of 1 μL of the sample was injected in split mode onto an Agilent split-mode liner that contained a small plug of silanized glass wool with the temperature set to 280° C. For all samples, one injection was made at split ratio of 20:1. A 30 m DB-5 ms GC column was used for chromatography. The oven temperature was initially held at 165° C. for 0.5 min. Then, the temperature increased 10° C./min up to 265° C., followed by an immediate increase of 30° C./min to 325° C., where it was kept constant for 3 min. The total run time was 15.5 min. The temperature of the transfer line was kept at 250° C. After the sample components were eluted from the GC column, they were subjected to electron ionization with an electron energy of 70 eV at a temperature of 250° C. The m/z range of analysis was 40-800 with a spectral acquisition rate of 10 Hz. Perfluorotributylamine was used for the daily tuning and calibration of the mass spectrometer.

Data Processing. Quantification was done by integrating the summed extracted ion chromatogram peak areas (details provided elsewhere)” using QuanLynx software. The peaks were integrated automatically and verified manually. Then, all the information given by integration was exported to a spreadsheet for further analysis.

Statistical Analysis. All data (chromatographic peak areas) for each sample analyzed as part of this example are provided within a spreadsheet available as the Supporting Information accompanying Ferdosi S. et al., Journal of Proteome Research 2018, 17(1):543-558. The peak area for each glycan node was normalized in one of two possible ways. In the first approach, individual hexoses were normalized to heavy glucose, and individual HexNAcs were normalized to heavy N-acetyl glucosamine (heavy GlcNAc). (Notably, these two internal standards were omitted during analysis of the prostate cancer set of samples.) In the second approach, individual hexoses were normalized to the sum of all endogenous hexoses, and individual HexNAcs were normalized to the sum of all endogenous HexNAcs. This normalization scheme provided modestly improved within-batch reproducibility but limited observation of potential simultaneous increases in all glycan nodes; see the spreadsheet provided in the Supporting Information accompanying Ferdosi S. et al., Journal of Proteome Research 2018, 17(1):543-558 “Average CVs” worksheet for details on the reproducibility of each normalization approach. Based on the QC sample analyzed in each batch, the average percent CV for the heavy glucose/heavy GlcNAc normalization approach for the top five performing glycan nodes described in the Results section was 17%; for normalization by the sum of endogenous hexoses or HexNAcs, this value was 10%.

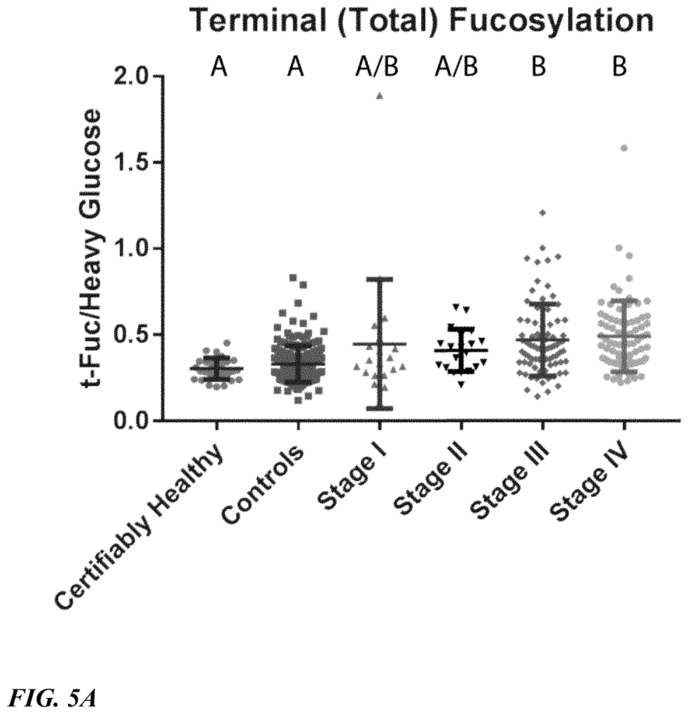

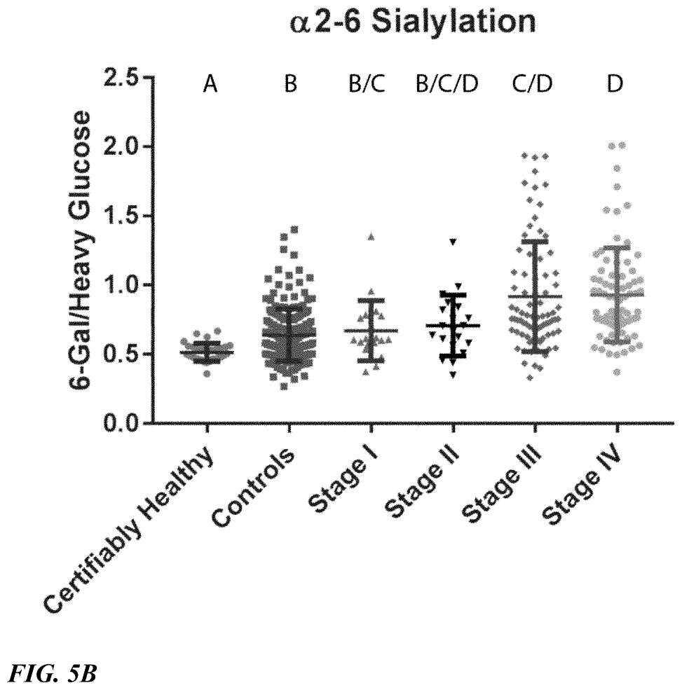

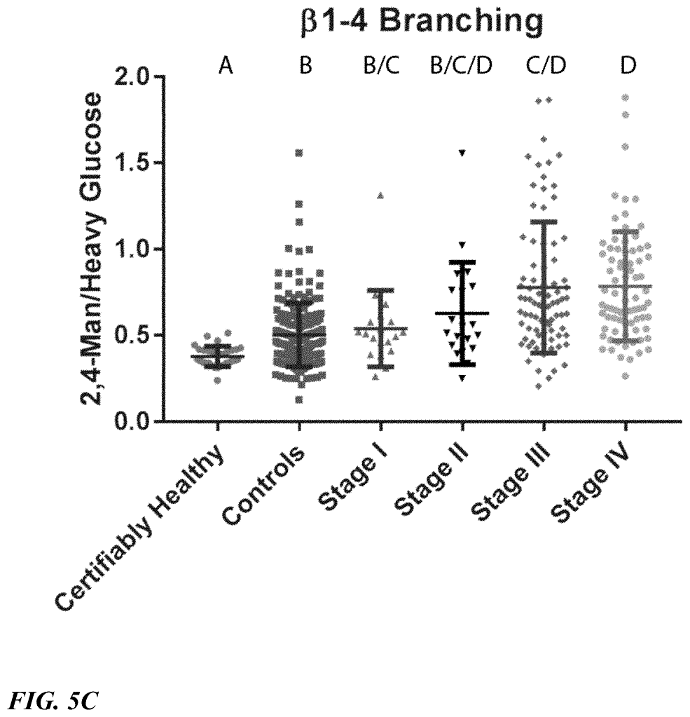

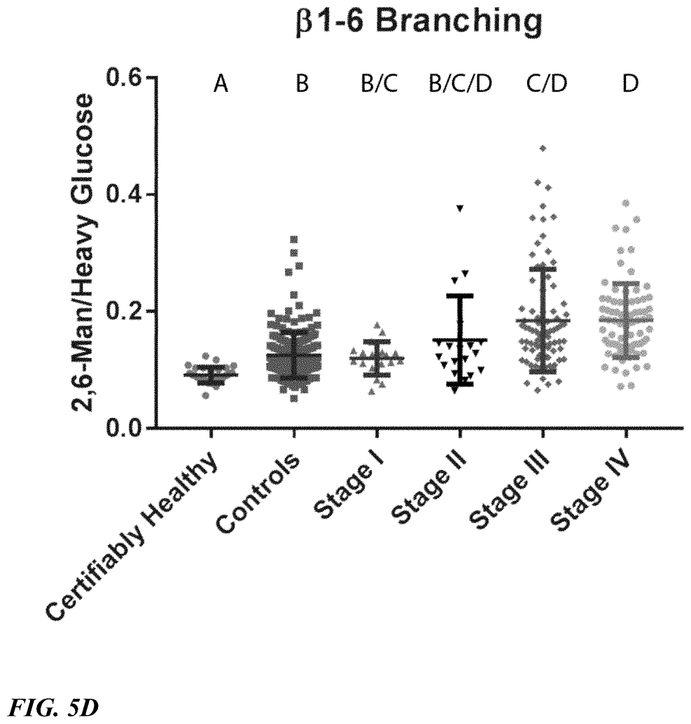

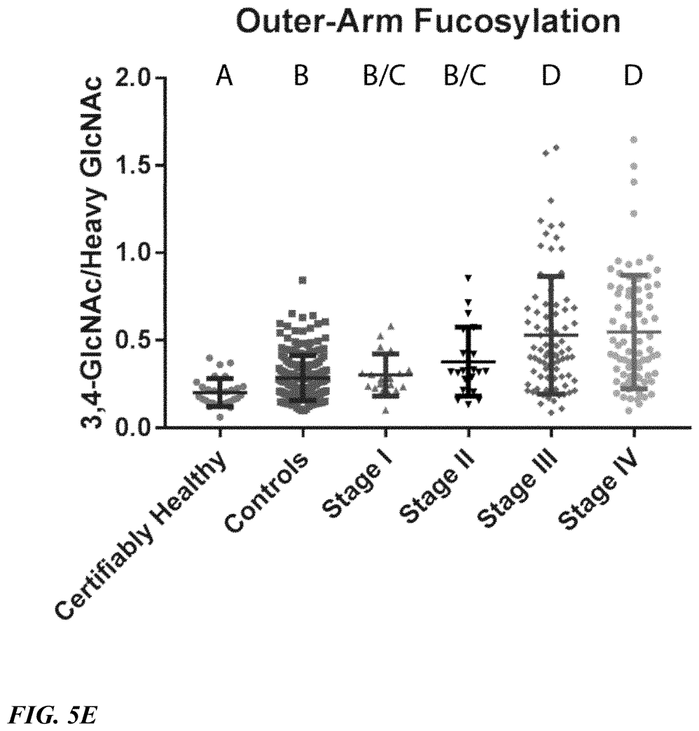

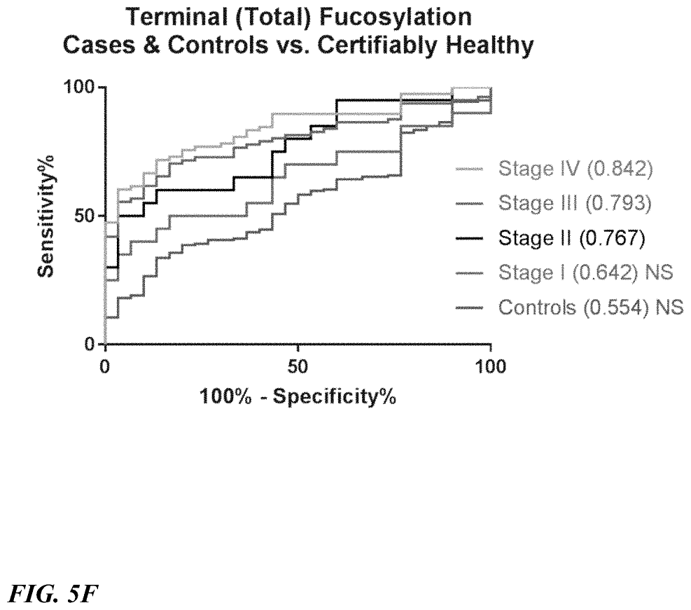

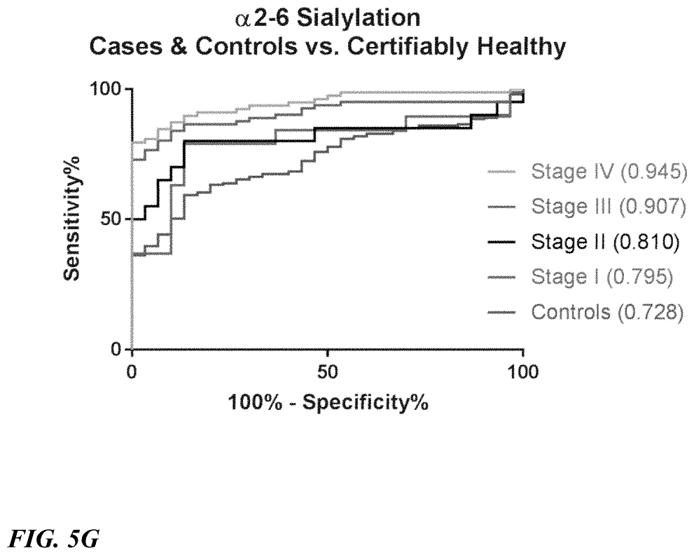

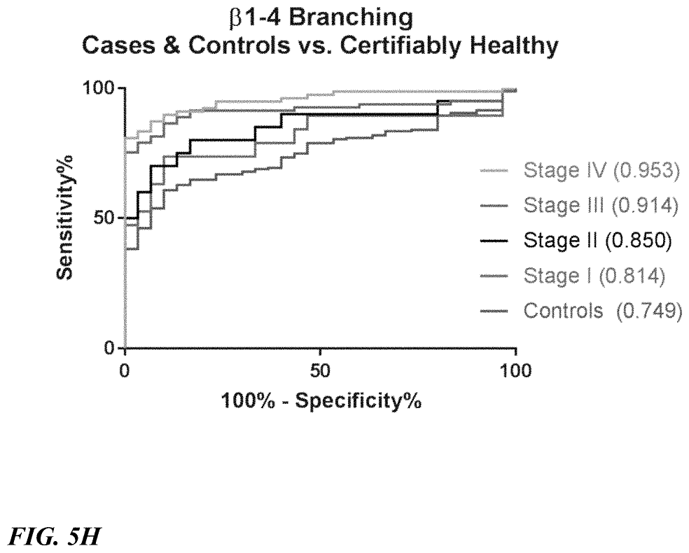

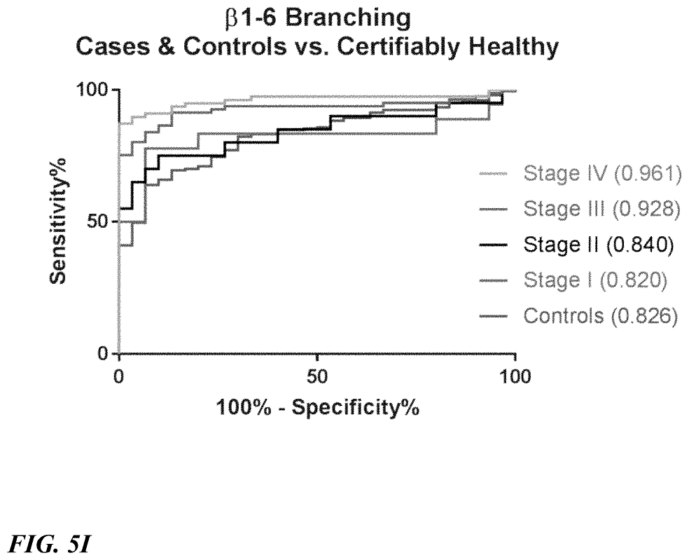

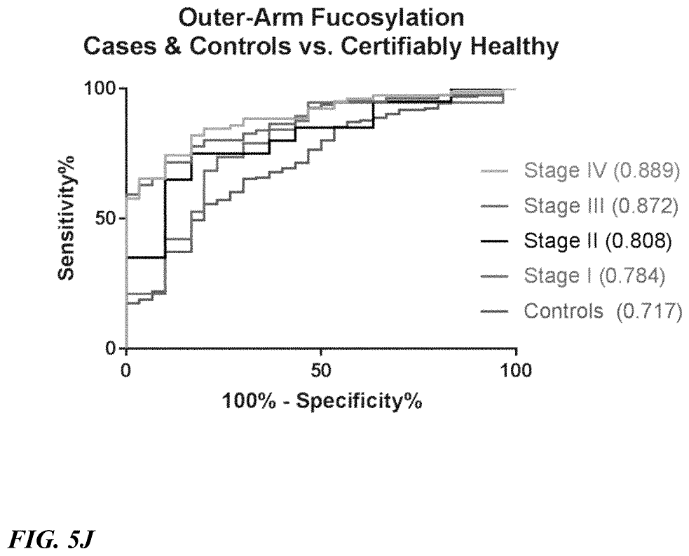

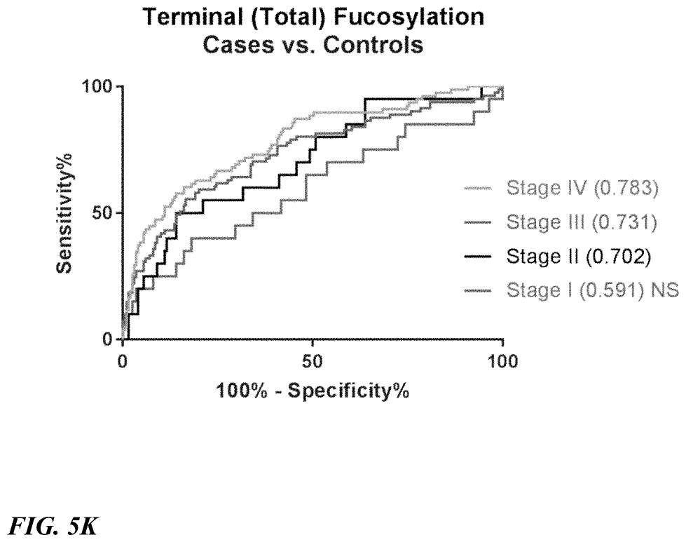

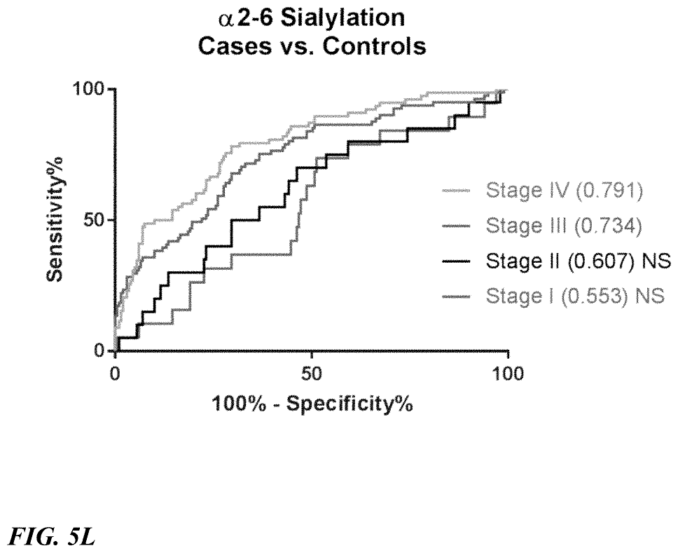

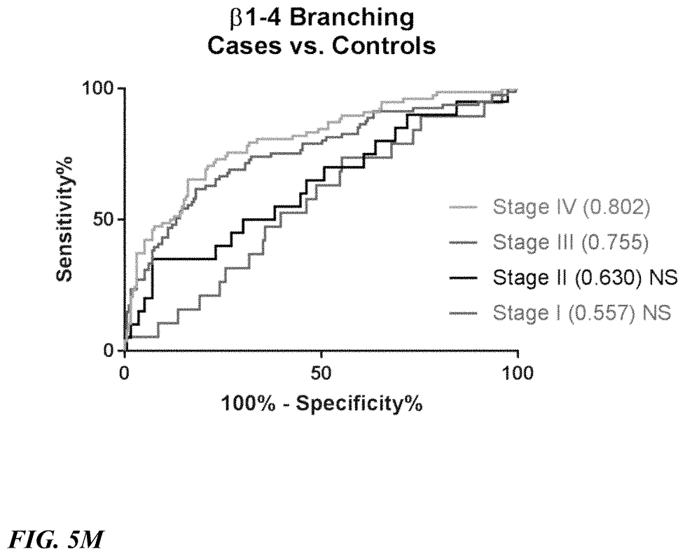

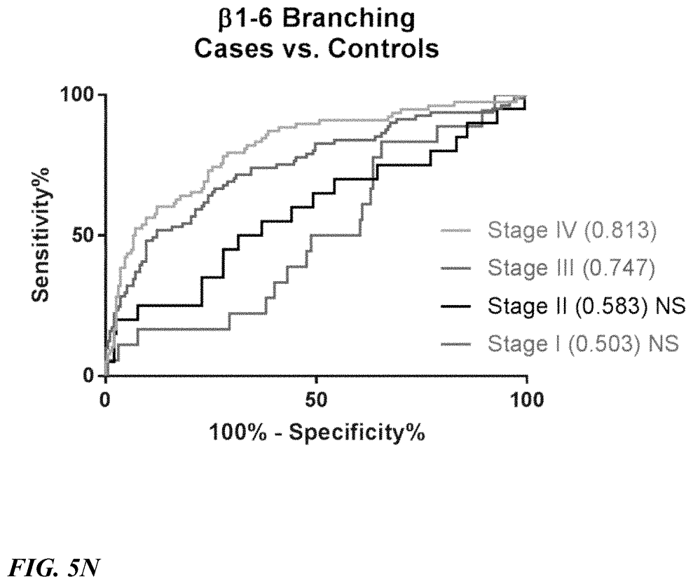

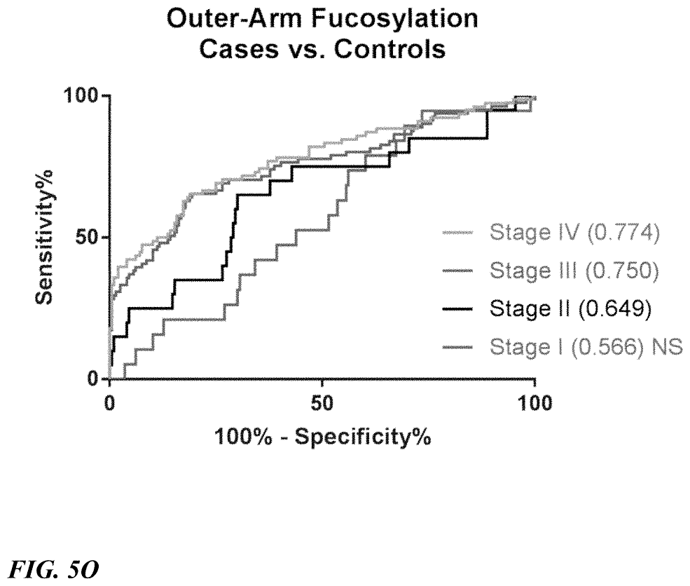

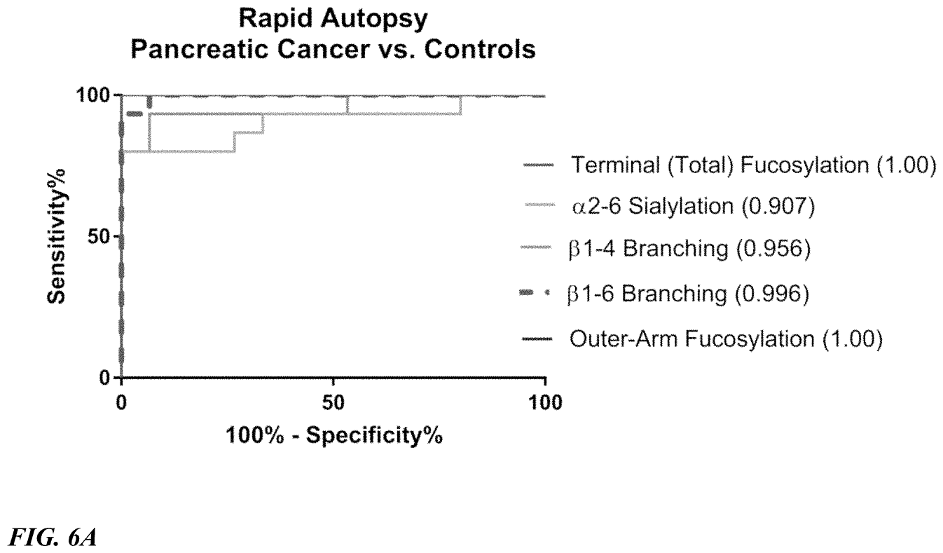

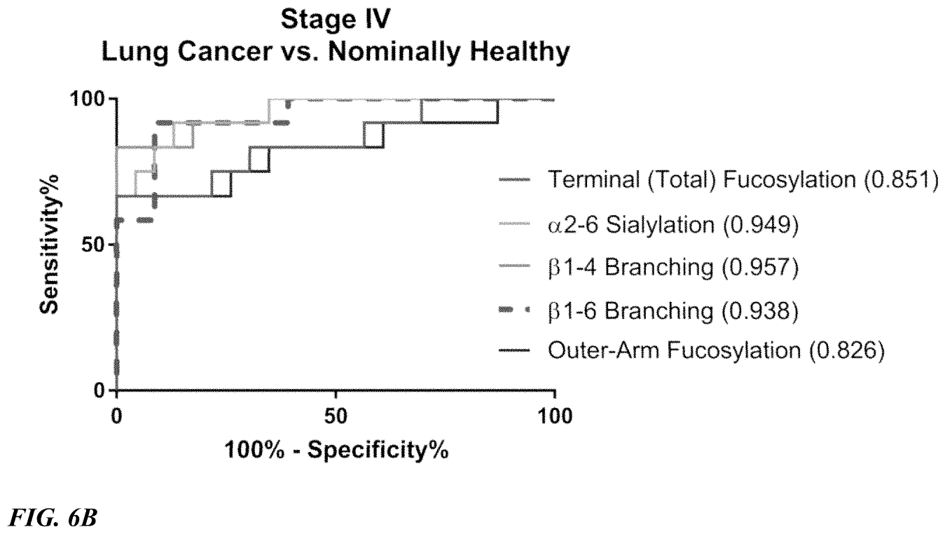

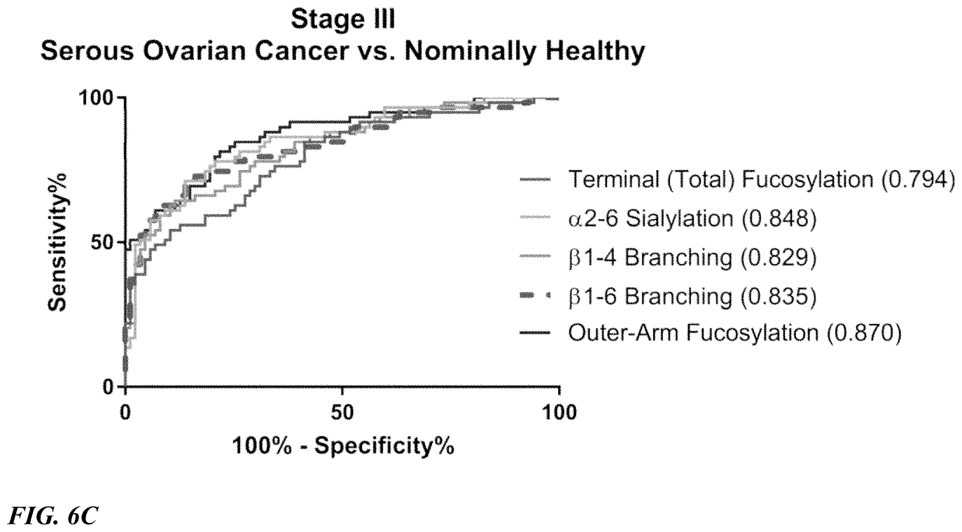

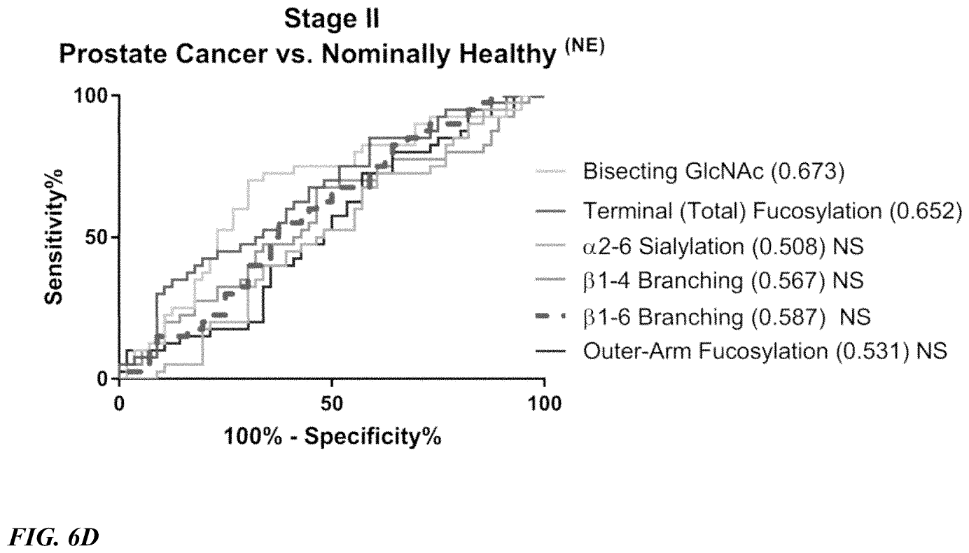

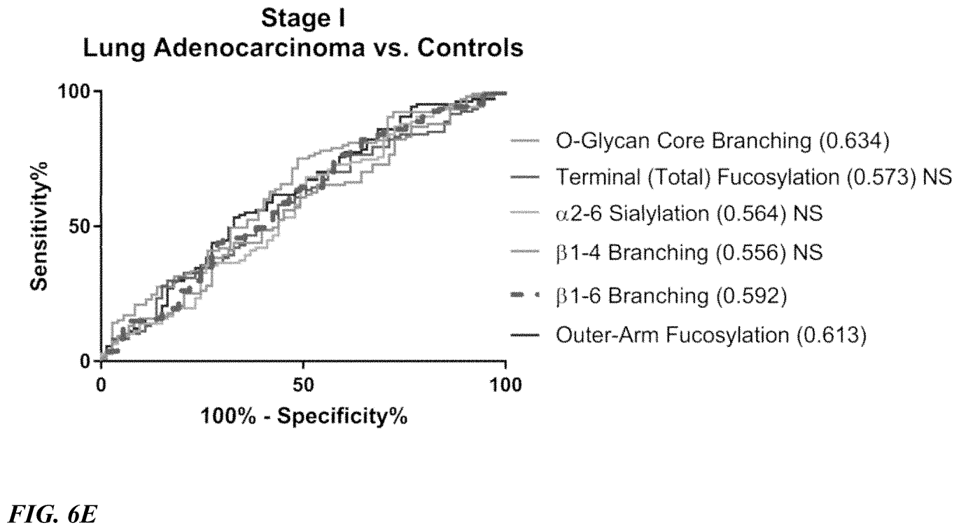

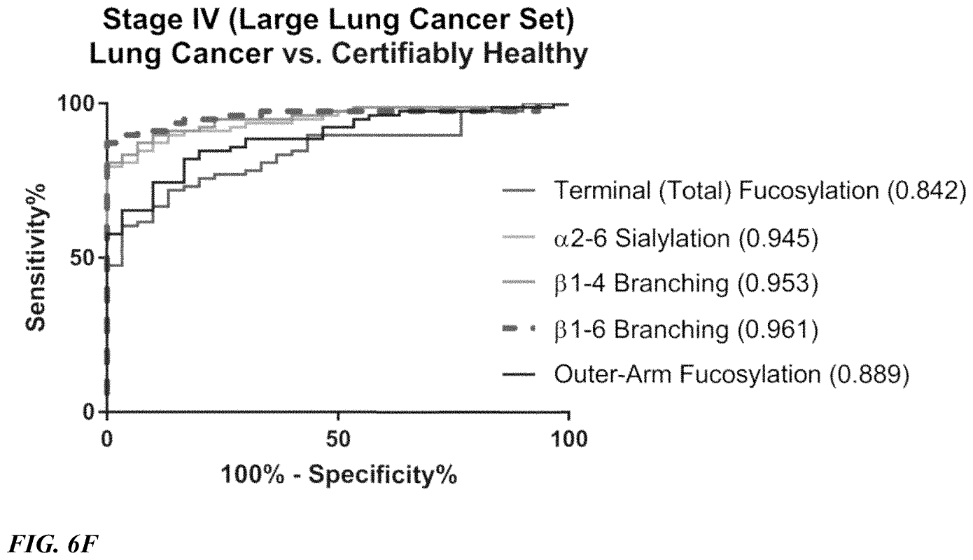

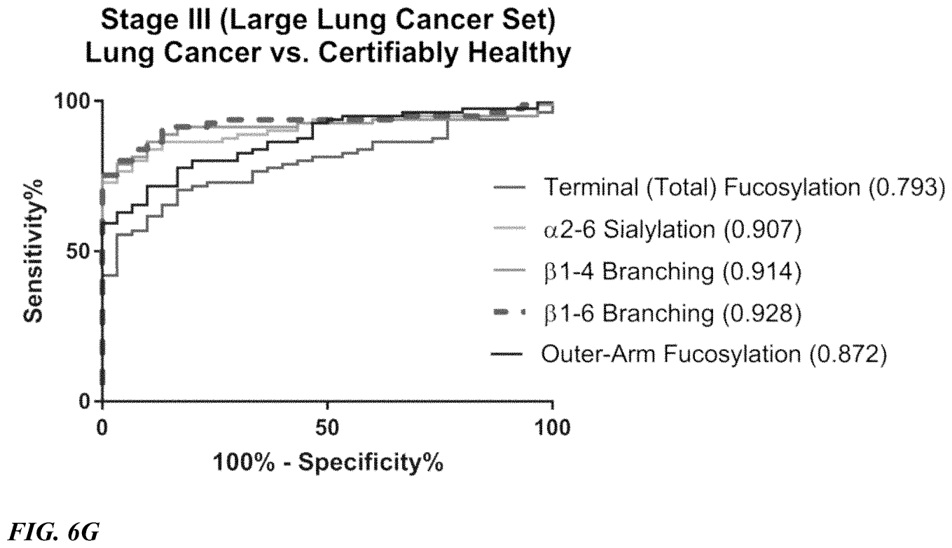

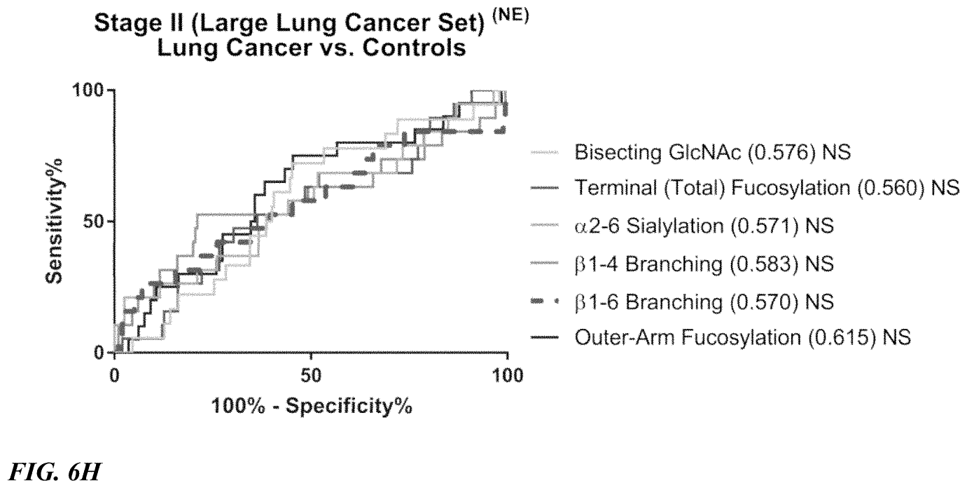

Each stage of each cohort was log-transformed, and outliers were removed with the ROUT method at Q=1% using GraphPad Prism 7. Data were then reversed transformed by taking the anti-log of each value. Differences between patient cohorts and stages in the large lung cancer study were evaluated by means of the Kruskal-Wallis test followed by the Benjamini-Hochberg false discovery correction procedure using R version 3.3.3. This software was also used to generate the receiver operating characteristic (ROC) curves that were statistically compared to one another via DeLong's test using RStudio Version 1.0.143. GraphPad Prism 7 was used to plot the ROC curves shown in FIGS. 5-6. Stage-by-stage multivariate modeling on the large lung cancer set was carried out using multivariate logistic regression, with performance assessed by leave-one-out cross-validation, and model selection was carried out using a best subsets procedure. These analyses were carried out using R version 3.3.3. The ability of particular glycan nodes to predict cancer progression and survival was assessed via Cox proportional hazards regression models using XLSTAT Version 2012.3.01, the results of which were verified (duplicated) using SAS 9.4. Survival curves were generated and associated log-rank Mantel-Cox tests were carried out using GraphPad Prism 7.

### Results

Prior to initiating this study, matched collections of serum and several different types of plasma were acquired from healthy donors. Glycan nodes were analyzed in these samples to determine whether subtle differences in sample matrix (i.e., different anticoagulants and serum) impacted the analytical results. Only a few statistically significant differences between the P/S matrices were observed (Tables S2 and S3 accompanying Ferdosi S. et al., Journal of Proteome Research 2018, 17(1):543-558). Sodium citrate and sodium EDTA plasma samples were excluded from this study, which accounts for all of the pair-wise differences observed in Table S2; a few remaining differences (noted within the smaller sample set involving heparin plasma; Table S3), while statistically significant, were small and actually within the interassay precision range for the relevant markers.¹⁹ A summary of all sample sets analyzed as part of this example is provided in FIG. 3.

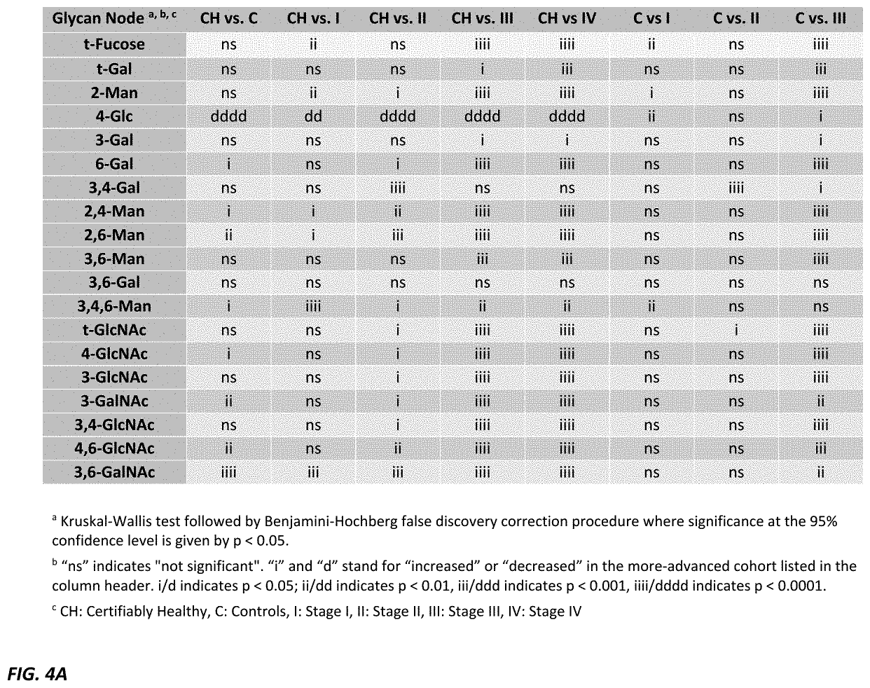

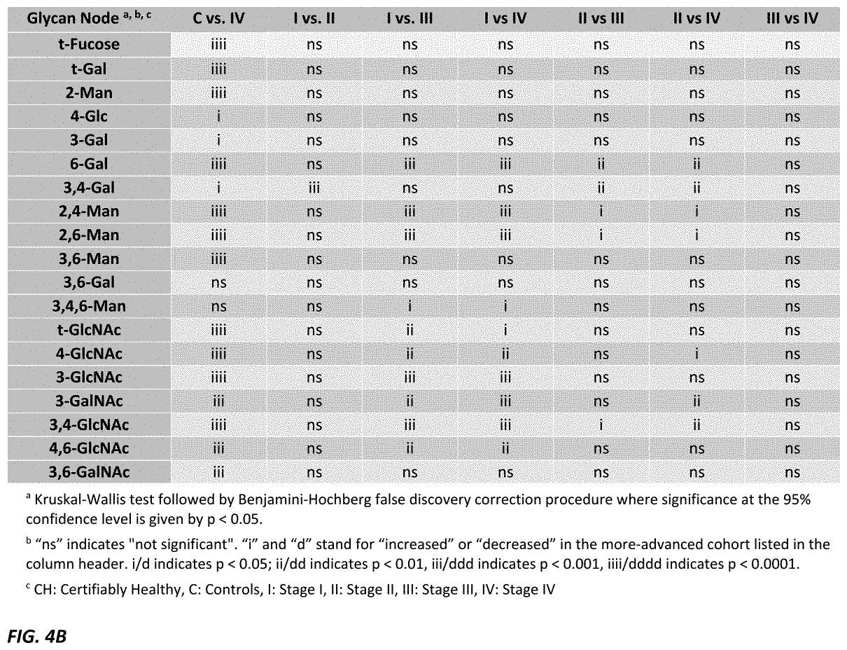

The primary focus of this study was the large lung cancer set as it constituted the single largest set and covered all stages of cancer. In total, 19 glycan “nodes” were measured with relative abundances that were consistently greater than 1% of respective total hexoses or total N-acetylhexosamines (HexNAcs). As reported elsewhere, this threshold ensures quantitative precision between batches of samples.^{18,19} Relative to the age/gender/smoking-status matched controls, significant changes were observed in 4 out of 19, 2 out of 19, 17 out of 19 and 17 out of 19 nodes in plasma samples taken from stage I, II, III and IV patients, respectively (FIG. 4A-FIG. 4B). Based on normalization to heavy, stable isotope-labeled glucose and GlcNAc internal standards, all altered glycan nodes except 4-Glc (which is mostly derived from glycolipids) were elevated in the cancer patients relative to the controls. Analogous results for data in which each hexose was normalized to the sum of endogenous hexoses and each HexNAc was normalized to the sum of endogenous HexNAcs revealed that this alternate normalization procedure is not as effective at teasing out differences between the cohorts in the large lung cancer study (Table S4 accompanying Ferdosi S. et al., Journal of Proteome Research 2018, 17(1):543-558).

Highly Altered Glycan Features: The five glycan nodes that were most elevated in the cancer cases relative to the at-risk controls included the following: 1) Terminal fucose-which corresponds to essentially all fucose in blood plasma. (Non-terminal fucose is only found in Notch proteins^{18,28} which, at most, would contribute only an infinitesimal fraction of the fucose found in blood plasma and, if ever detected by the approach employed here, would be observed as 3-linked fucose.) 2) 6-linked galactose, which corresponds specifically to α2-6 sialylation and almost completely to the activity of the ST6GalI glycosyltransferase enzyme¹⁸; 3) 2,4-linked mannose, which corresponds to β1-4 branching of N-linked glycans and almost completely to the activity of the GnT-IVa enzyme¹⁸; 4) 2,6-linked mannose, which corresponds to β1-6 branching of N-linked glycans and to the activity of the GnT-V enzyme¹⁸; 5) 3,4-linked N-acetylglucosamine (GlcNAc), which predominately corresponds to outer-arm fucosylation and the activity of the FucT-III, FucT-V, FucT-VI, and FucT-XI enzymes¹⁸. The univariate distributions of these five glycan nodes (normalized to heavy glucose or heavy GlcNAc added as an internal standard), along with receiver operating characteristic (ROC) curves that describe the potential clinical relevance of their distributions are shown in FIG. 5A-FIG. 5O.

Stage and Health-Status Dependence: FIG. 4A-FIG. 4B and FIG. 5A-FIG. 5O illustrate both the strong stage-dependence of these glycan features as well as the notable contrast of their distributions in certifiably healthy individuals compared to the general middle-aged to elderly population (i.e., “controls”) who are at a similar risk for cancer as individuals who actually had cancer. Similar distributions and trends were noted when the five glycan nodes were normalized to the sum of endogenous hexoses or HexNAcs, but the ROC c-statistics (areas under the curve, AUCs) tended not to be as large (FIG. S1 accompanying Ferdosi S. et al., Journal of Proteome Research 2018, 17(1):543-558). The average age of the certifiably healthy living kidney donor population was 47 and that of both the controls and lung cancer cases in this set of samples was 61 (Table S1 accompanying Ferdosi S. et al., Journal of Proteome Research 2018, 17(1):543-558). However, after pooling data from the certifiably healthy donors, controls and lung cancer cases and correcting for multiple comparisons, no statistically significant correlations with age were observed for any of these glycan nodes. (Before correcting for multiple comparisons, terminal (total) fucose appeared slightly correlated with age (Pearson correlation R²=0.013 and p=0.021), but this result cannot be considered statistically significant after considering the fact that multiple comparisons were made.) Likewise, no statistically significant correlations of glycan nodes with age were observed when these groups of patients were evaluated individually.

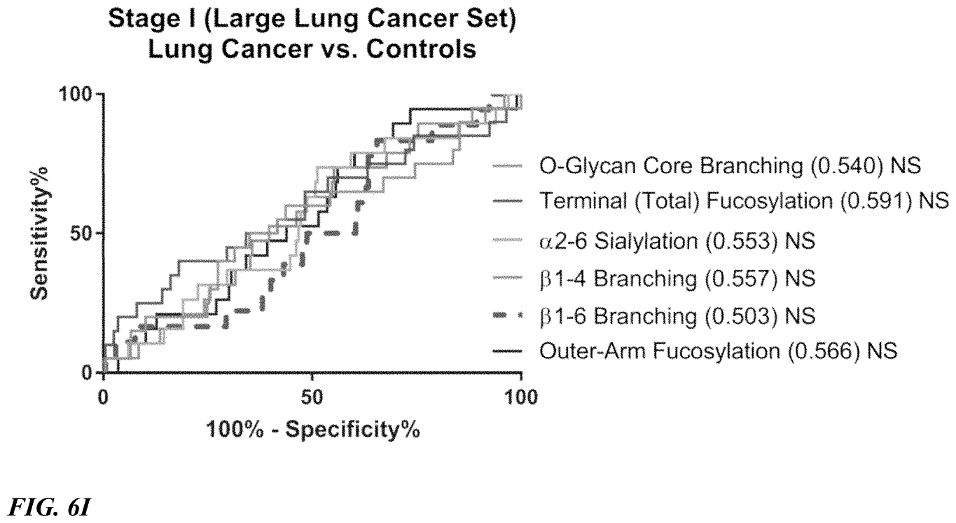

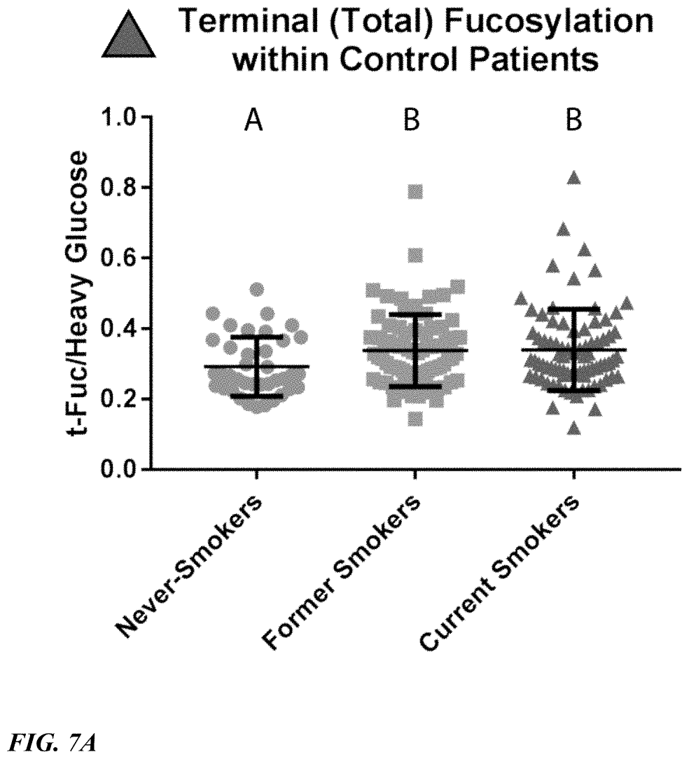

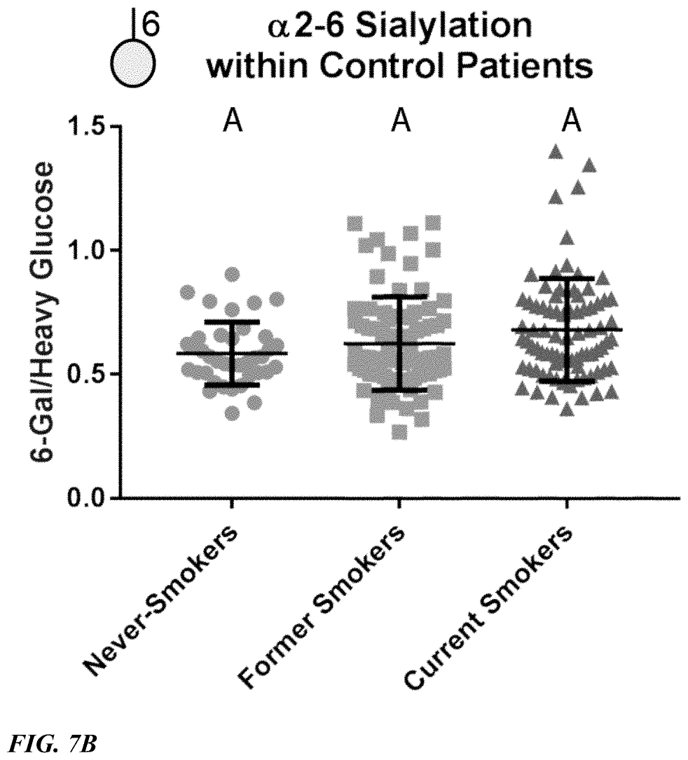

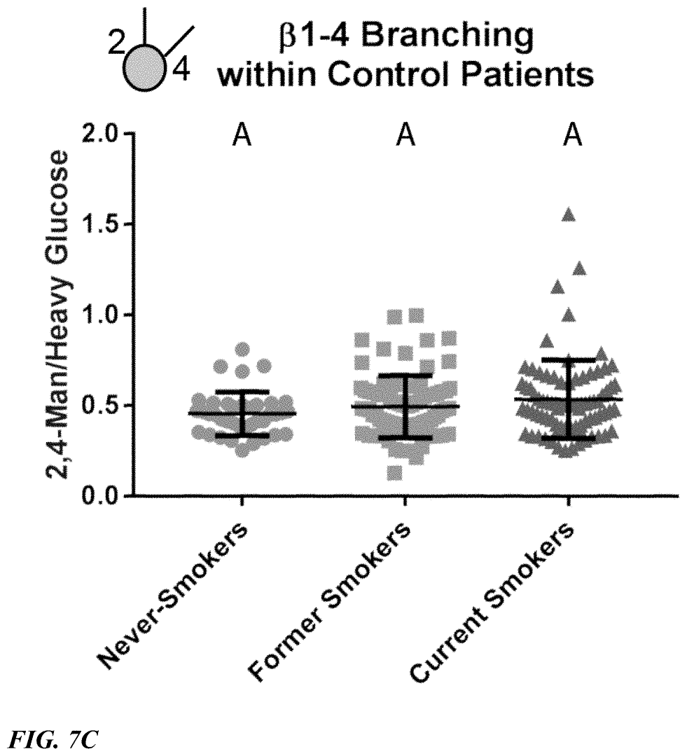

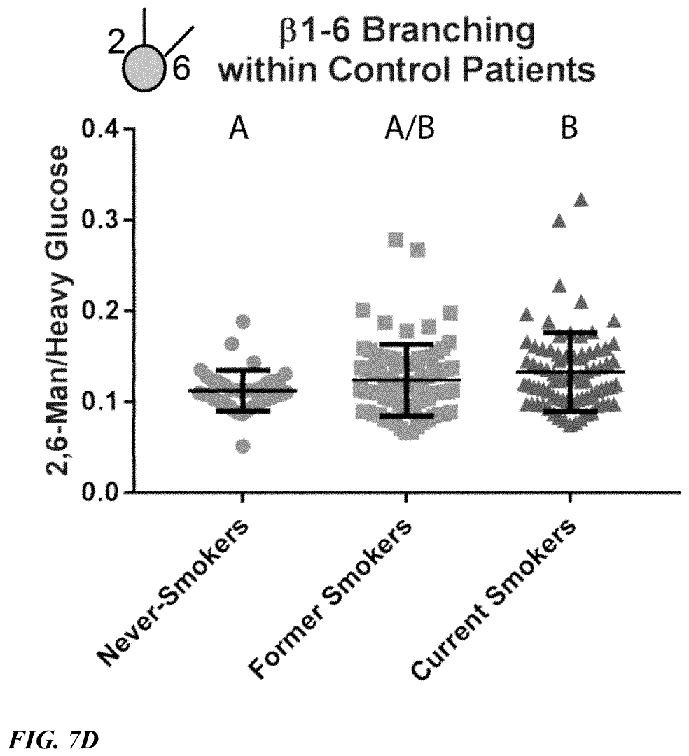

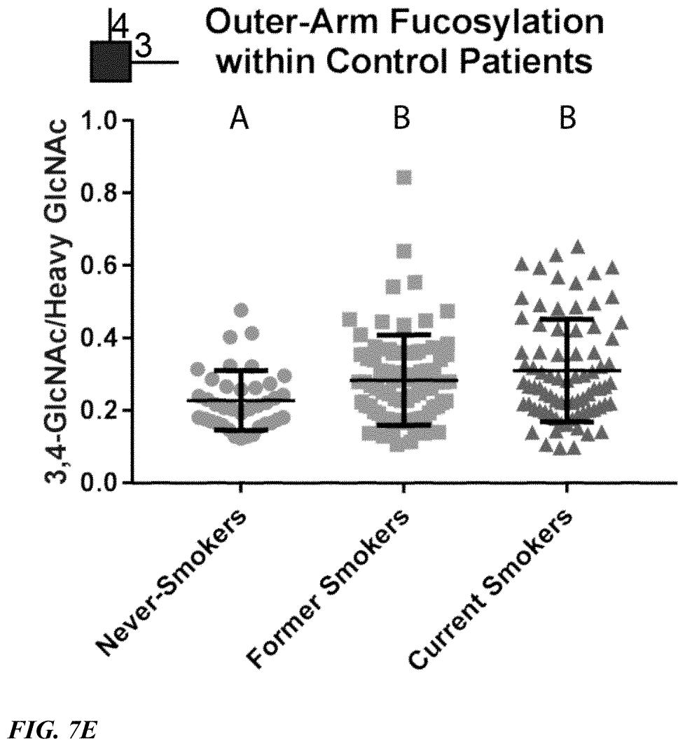

In general, the five glycan nodes increased together as the stage of cancer advanced (FIG. 6A-FIG. 6I). Moreover, the behavior of these nodes was independent of the organ of tumor origin—at least when comparing lung cancer with pancreatic, ovarian and prostate cancers. This was quantitatively evident when the ROC curves in panels c and d of the left column of FIG. 6A-FIG. 6I were statistically compared with ROC curves from their respective lung cancer stages shown in the right column (Table S5 accompanying Ferdosi S. et al., Journal of Proteome Research 2018, 17(1):543-558). At stage IV both outer-arm fucosylation and terminal (total) fucosylation lag a bit behind α2-6 sialylation, β1-4 branching and β1-6 branching—but fucosylation-related nodes caught up and even surpassed these other glycan features once cancer had fully run its course (FIG. 6A). When stage was held constant, no glycan nodes were found to be significantly different between adenocarcinoma, squamous cell carcinoma and small cell carcinoma—the three different histological sub-types observed in the large lung cancer study (Table S1 accompanying Ferdosi S. et al., Journal of Proteome Research 2018, 17(1):543-558). However, terminal (total) fucosylation, β1-6 branching, and outer-arm fucosylation were altered within the control cohort on the basis of smoking status (grouped as never-smokers, former smokers or current smokers) (FIG. 7)

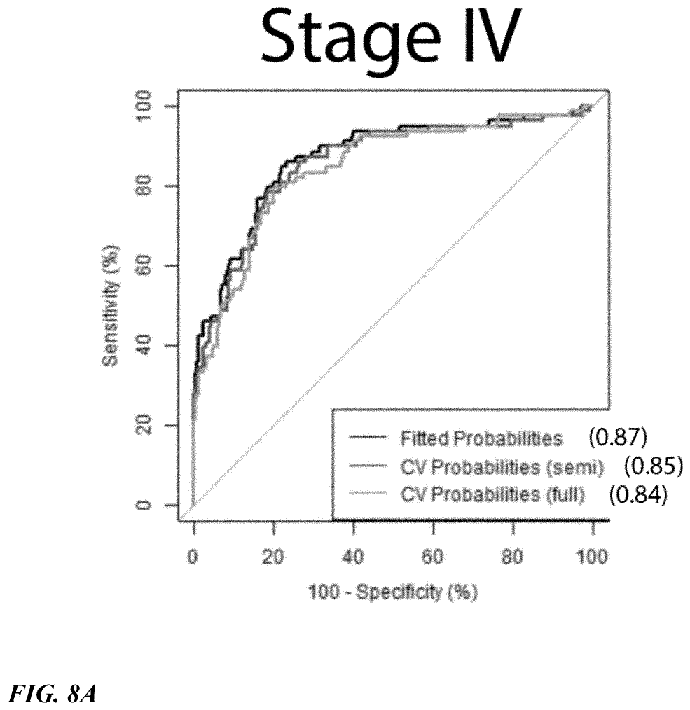

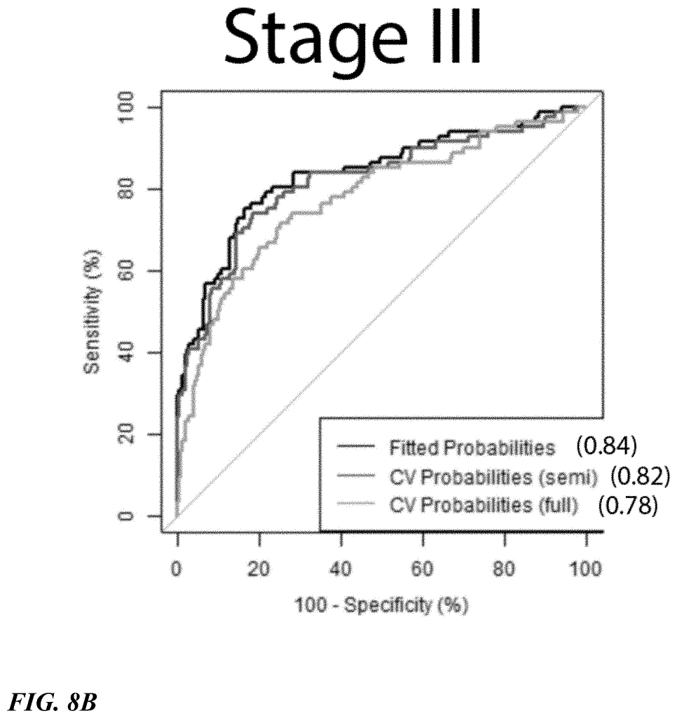

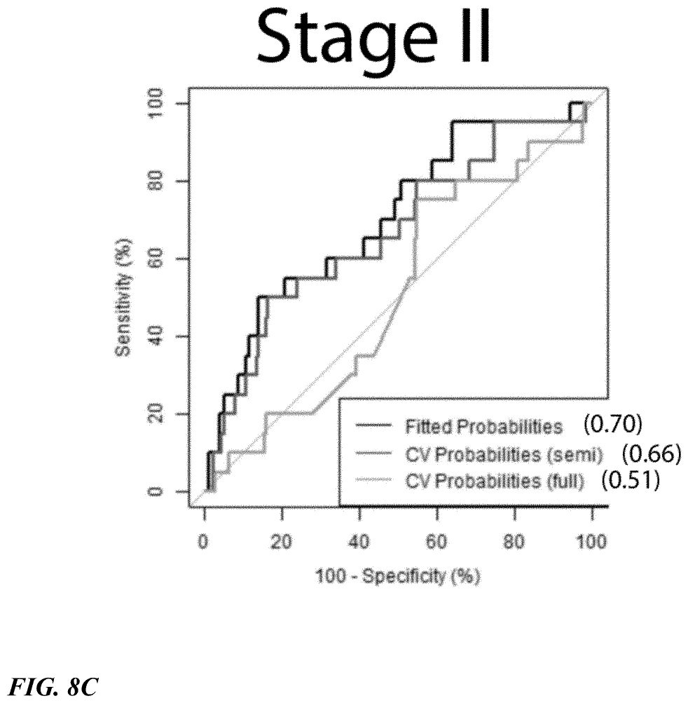

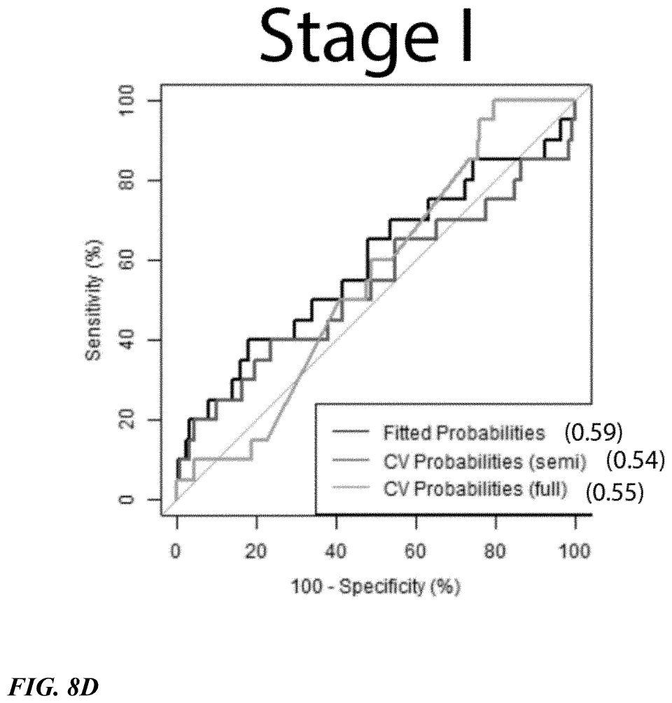

Orthogonality of Glycan Features: In order to evaluate the orthogonality of all 19 glycan nodes included in this study (FIG. 4A-FIG. 4B), multivariate logistic regression models were created for the large lung cancer set on a stage-by-stage basis (FIG. 8A-FIG. 8D). Results of modelling are shown as ROC curves, where the model-derived predicted probability of disease for the sample was used as the discriminatory variable. In summary, the fully cross-validated forms of these multivariate logistic regression models do not distinguish lung cancer cases from controls any better than individual glycan nodes (cf. FIGS. 6F-6I). This indicates a general lack of orthogonality or independence between the glycan features observed in this study.

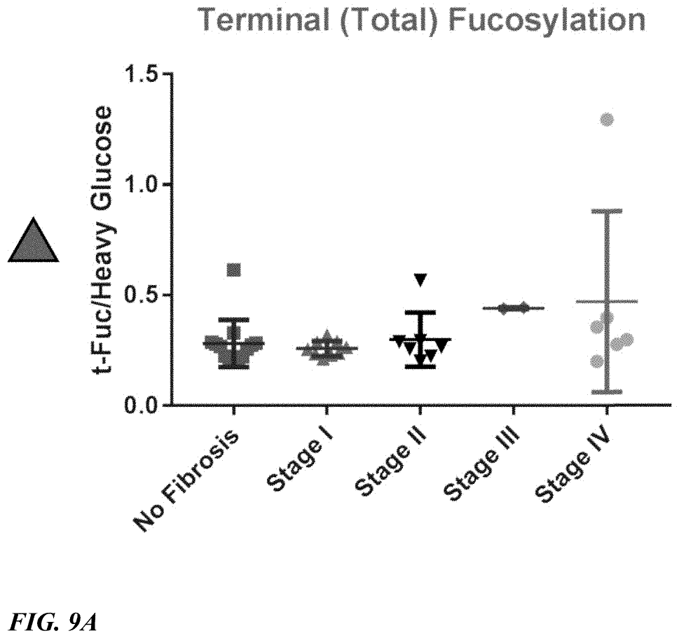

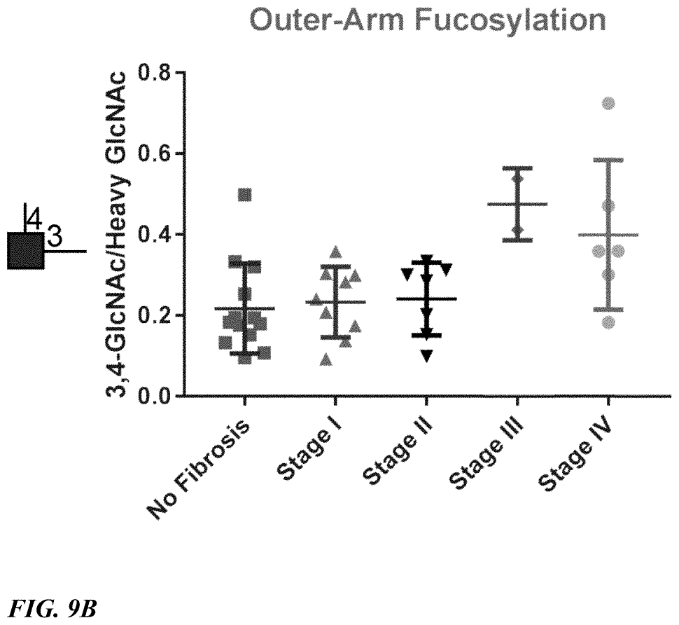

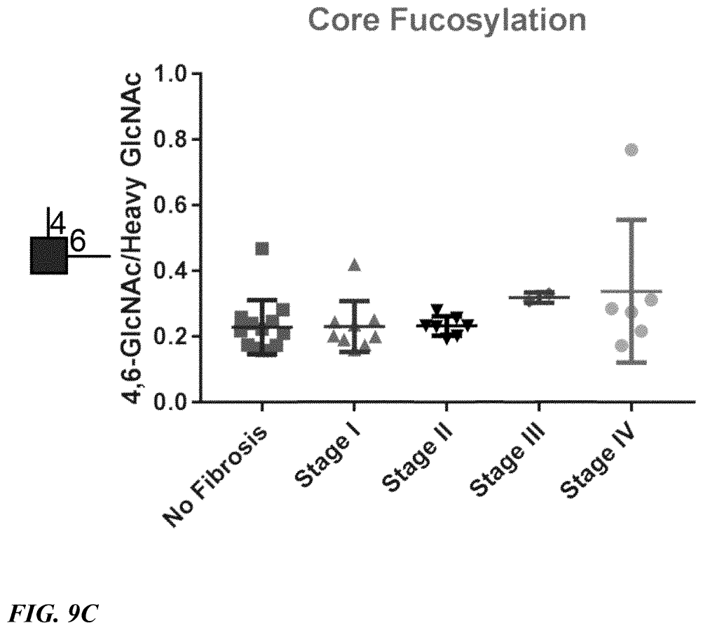

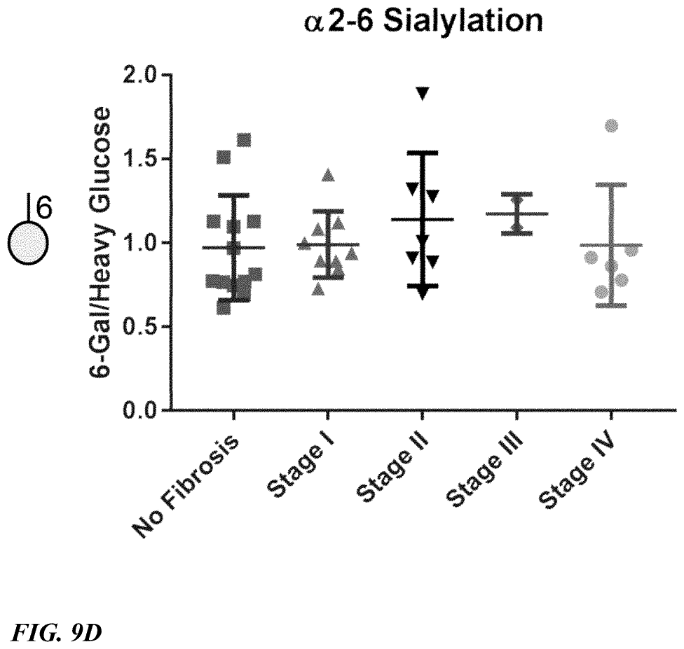

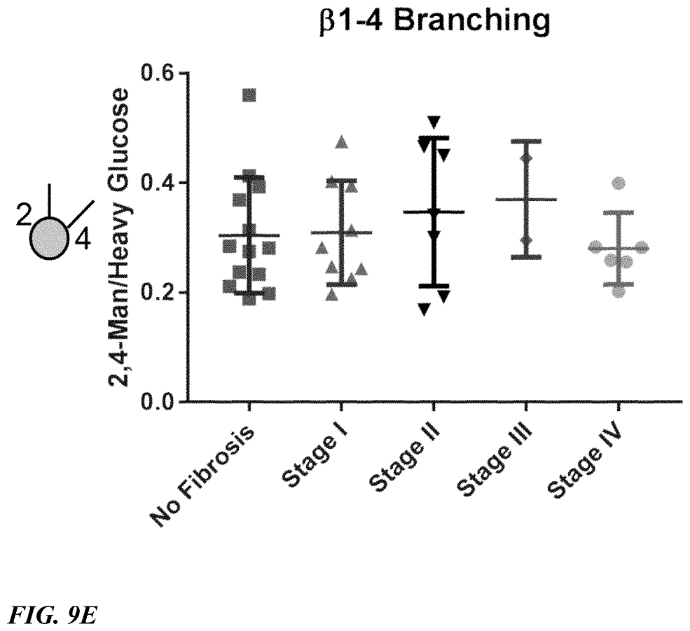

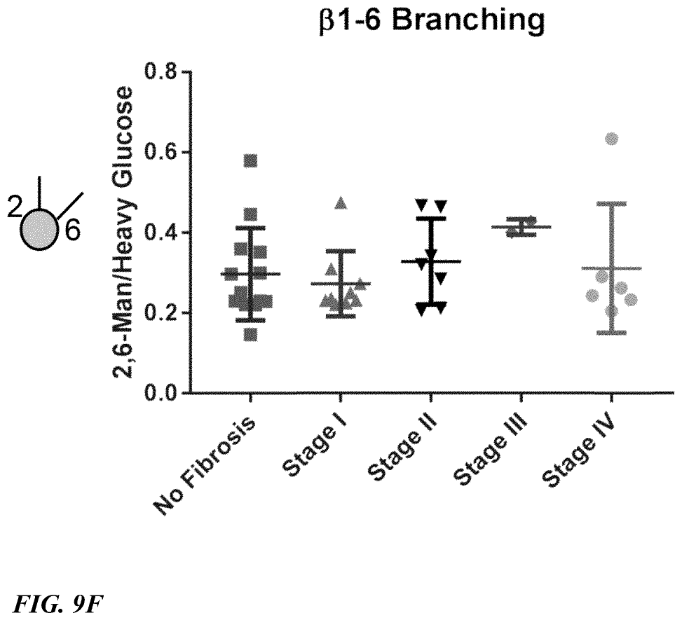

Comparison to Liver Fibrosis: The vast majority of glycoproteins found in blood P/S are derived from either liver glycoproteins or immunoglobulins (IgG molecules) secreted by the immune system.^{29,30} In terms of raw abundance, which is in the 10 s of mg/mL range, the relative contribution of P/S glycoproteins provided by the liver and by the immune system is approximately 50% each.³⁰ Essentially all non-protein targeting serum glycomics approaches, including the one employed in this study, detect changes in these abundant P/S glycans and not novel glycans secreted or sloughed-off by cancer cells. This concept has been acknowledged elsewhere.³¹ Nevertheless, P/S glycans are notoriously known for being altered in cancer.^{1-4,32} However, they are also known to be altered in inflammatory conditions in the absence of cancer.³³⁻³⁵ As an initial attempt to begin to parse out the behavior of the five glycan nodes that were most elevated in the large lung cancer set, they were analyzed in a set of serum samples from liver fibrosis patients (FIG. 9A-FIG. 9F). Statistical analysis (Kruskal-Wallis) indicated that there were no significant differences in any of the glycan nodes shown across all stages of liver fibrosis. This may have been due to limited statistical power. Notably, however, fucosylation-related markers exhibited a tendency to be elevated in stage III-IV liver fibrosis.

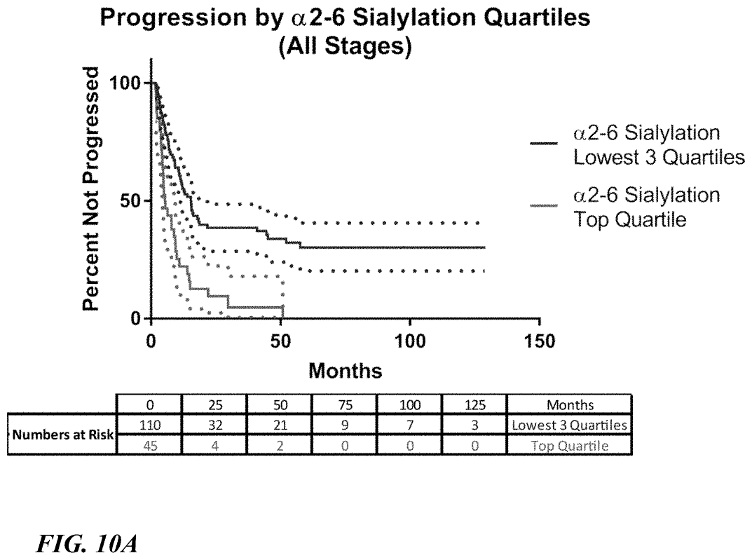

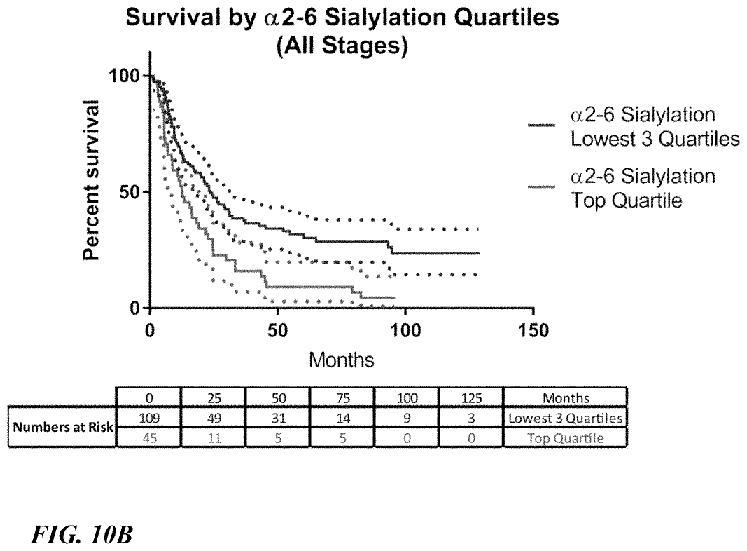

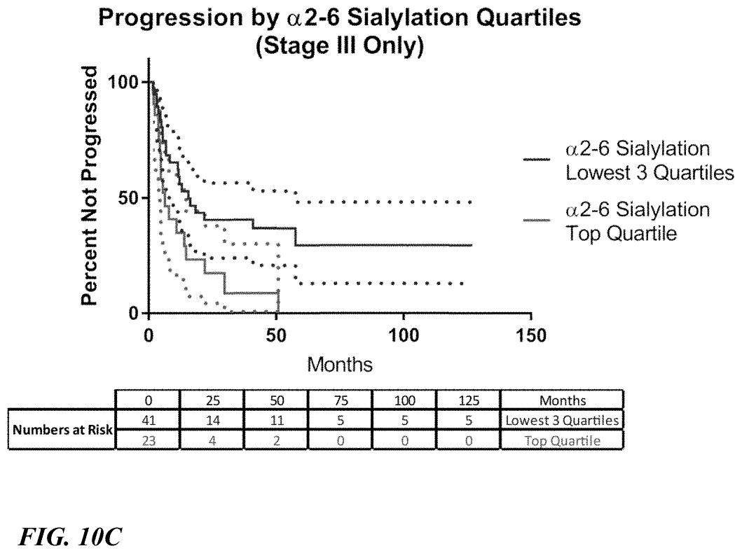

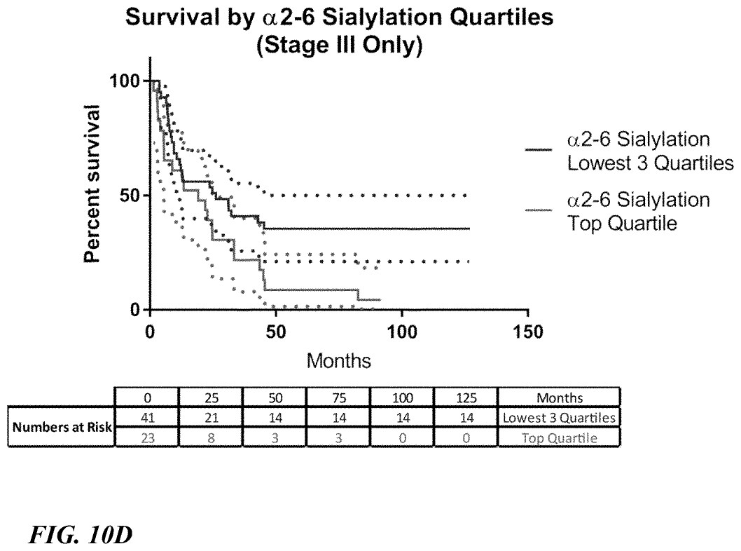

Prediction of Progression and All-Cause Mortality: The five glycan nodes that were most elevated in the large lung cancer set were evaluated for their ability to predict both progression and all-cause mortality in a Cox proportional hazards regression model. After adjusting for age, gender, smoking status and cancer stage, only 6-linked galactose, which corresponds to α2-6 sialylation, predicted both progression and all-cause mortality with p-values of <0.01 when the glycan nodes were modeled as continuous variables. All four other top-performing glycan nodes were able to predict survival (p<0.05), but only β1-4 branching and β1-6 branching were also able to predict progression (p<0.05). Because relative rather than absolute quantification was employed, glycan node units lack readily interpretable meaning. As such, measurements of α2-6 sialylation were broken into quartiles and the Cox proportional hazards analysis repeated. After adjusting for age, gender, smoking status and cancer stage, the top α2-6 sialylation quartile predicted progression with a hazard ratio of 2.45 relative to all other quartiles combined (lower bound at 95% CL=1.54; upper bound at 95% CL=3.90; p=1.5×10⁻⁴). Likewise, after the same adjustments, the top α2-6 sialylation quartile predicted all-cause mortality with a hazard ratio of 1.52 relative to all other quartiles combined (lower bound at 95% CL=1.02; upper bound at 95% CL=2.23; p=0.042). Progression and survival curves illustrate the differences in the rates of occurrence of these events for the top α2-6 sialylation quartile vs. all other quartiles (FIG. 10A-FIG. 10D). Progression and survival curves for stage III patients alone illustrate that the separation of progression by α2-6 sialylation and the separation of survival by α2-6 sialylation is not simply driven by stage (FIG. 10C-10D). α2-6 sialylation was not elevated or able to predict progression or survival in the stage I lung adenocarcinoma set.

### Discussion

The five glycan features that were most elevated relative to healthy individuals and at-risk controls were terminal (total) fucosylation, α2-6 sialylation, β1-4 branching, β1-6 branching and outer-arm fucosylation (FIGS. 4A-4B, 5A-5O, and 6A-6I). Two phenomena stood out most with regard to their distributions amongst the cohorts of the large lung cancer study: First, there was a striking stage dependence of all five glycan features that was independent of the tumor organ of origin (FIGS. 4A-4B, 5A-5O, and 6A-6I, and Table S5 accompanying Ferdosi S. et al., Journal of Proteome Research 2018, 17(1):543-558). In part, statistical significance at earlier stages may not have been achieved due to the relatively low number of samples measured from patients at stages I-II (n˜20 per stage). Statistically significant elevation of core-branched O-glycans (i.e., 3,6-linked GalNAc) over age/gender/smoking-status matched controls was observed in stage I lung adenocarcinoma from this separate, larger set of samples (FIG. 6A-FIG. 6I). However, it was clear from the ROC curve (FIG. 6E) that this glycan node cannot serve as a useful early stage diagnostic biomarker.

A second notable feature apparent in the large lung cancer set was the statistically significant difference between certifiably healthy living kidney donors and risk-matched controls for α2-6 sialylation, β1-4 branching and β1-6 branching—with controls always increased toward the direction of cancer (FIGS. 4A-4B and 5A-5O). These differences between certifiably healthy individuals and patients with an elevated risk of cancer underscore the high risk of false discovery when nominally healthy sample donors rather than well-characterized, clinically relevant controls are employed during biomarker development. The notable differences between healthy individuals and at-risk controls also supports the idea that the biological landscape within plasma/serum may undergo “grooming”, “conditioning” or pre-metastatic “niche” formation prior to cancer taking hold within the body.³⁶⁻⁴⁰ Given that inflammation is closely tied to the development of cancer^{41,42} and that at least some glycans and glycan features are known to be altered in inflammatory conditions in the absence of cancer³³⁻³⁵, pre-cancerous inflammation may be responsible for the elevation of many of the glycan features observed in the at-risk controls relative to the certifiably healthy living kidney donors-suggesting that the goal of preventing such a pre-cancerous state may be as important as preventing the transition from an at-risk state to stage I cancer. With this in mind, it is interesting to note that about 62% of the age-qualified U.S. population would be excluded as living kidney donors due to preventable health conditions.⁴³

A few studies have been published that are closely related to the one reported here, but in which intact glycans were analyzed.^{31,44,45} While not in conflict with any of these studies, our most prominent findings of increased terminal (total) fucosylation, α2-6 sialylation, β1-4 branching, β1-6 branching and outer-arm fucosylation in stage III-IV lung cancer are most closely aligned with the major changes reported by Vasseur et al.³¹ for intact glycans in lung cancer. They reported significant increases in fucosylated tri- and tetra-antennary structures, outer-arm fucosylated structures, and α2-6 sialylated structures. Moreover, they reported that all of these features were elevated in control-group former smokers relative to control group non-smokers. We found increases in terminal (total) fucosylation, β1-6 branching and outer-arm fucosylation in current smokers relative to never smokers, but only increases in terminal (total) fucosylation and outer-arm fucosylation in former smokers relative to never smokers (FIG. 7A-FIG. 7E). Notably, the methods employed for the analysis of intact glycans by Vasseur et al. is one of just a few approaches that are capable of distinguishing 6-linked from 3-linked sialic acid.⁴⁶⁻⁴⁸

Multivariate logistic regression models were not able to outperform individual glycan nodes (cf FIG. 6F-I and FIG. 8A-D) with regard to distinguishing cancer at stages I-IV from controls. This indicates a general lack of biological orthogonality amongst the abnormal glycan features observed—suggesting that they likely have a singular (or small set of closely related) upstream causes: The concentration of glycoproteins in P/S is in the tens of milligrams per milliliter. As such, the observations of significant changes in P/S glycans observed here cannot be due to glycoproteins shed directly from cancer cells; almost certainly they are derived from alterations to one or both of the two major sources of P/S glycoproteins—namely liver glycoproteins or immunoglobulins (IgG molecules) secreted by the immune system.^{29,30} Such alterations are thought to be mediated by cytokines secreted from the tumor microenvironment and exist as part of an acute-phase inflammatory response.^{31,49-53}

But this is not to imply that liver glycoprotein and immunoglobulin glycan alterations are unimportant or lack a cancer-relevant pathological effect. Several cancer-upregulated glycoforms that cancer cells have in common with glycans that are induced on acute phase liver proteins and/or IgG molecules in the presence of cancer have been found to mediate specific immune-modulating effects—some of which overtly favor cancer progression:

Galectins are a family of lectins that bind β-galactoside sugars within glycans and are known to modulate a variety of immunological processes involved in cancer.^{39,54,55} Malignant T-cells in mycosis fungoides/Sezary syndrome have been found to resist galectin-1 mediated apoptosis because they both lack the CD7 receptors that carry the oligosaccharides recognized by galectin-1 and because they express sialylated core 1 O-glycans that promote galectin-1 resistance.⁵⁶ Poly-N-acetylactosamine-modified core 2 O-glycans bind to galectin-3, reducing the affinity of tumor major histocompatibility complex (MHC) class I-related chain A (MICA) for the activating NKG2D receptor on natural killer (NK) cells, preventing tumor cell killing of core 2 O-glycan expressing cancer cells.⁵⁷⁻⁶⁰ Similarly, modification of MUC1 by poly-N-acetylactosamine and subsequent binding by galectin-3 interferes with TRAIL-mediated killing of DR4-expressing cancer cells by NK cells.⁶⁰⁻⁶² But perhaps the best known example is the ability of excessive tumor cell surface sialylation to continually stimulate the inhibitory Siglec-7 receptor on NK cells, preventing their activation.^{60,63-65}

In light of these discoveries, the fact that α2-6 sialylation of abundant plasma/serum proteins is both associated with metastasis and poor prognosis^{66,67} and, in our study, was not only elevated in lung cancer but predicted progression and all-cause mortality in the large lung cancer set may shed additional light on a means by which cancer potentially manipulates the immune system to groom the physiological landscape and carve out a metastatic niche: Rather than directly interacting (cell-to-cell) with NK cells, tumor cells may simply be able to send out cytokine signals that are picked up by the liver and/or the immune system that alter the way that these nominally healthy tissues glycosylate their secreted proteins. This could, for example, facilitate a large-scale amplification of sialylated glycans that are able to continually activate Siglec-7 receptors on NK cells, preventing them from killing tumor cells and allowing them to metastasize. The possibility that cancer cells may induce the abnormal glycosylation of the highly abundant liver glycoproteins and/or IgG molecules found in P/S as a shielding mechanism against innate immune detection during metastasis attempts has received very little attention, but may be worth investigating. Though speculative, this strategy could even potentially be deployed in cases where cancer cells deplete themselves of a glycan feature required for immune-cell recognition—such as fucosylation recognized by the TRAIL-mediate killing mechanism of NK cells⁶⁸—but induce it on abundant P/S proteins, serving to “swamp out” the recognition mechanism of innate immune surveillance.

The ability of α2-6 sialylation to predict lung cancer progression and survival is not unique among P/S glycans. Indeed, all five top-performing glycan nodes in the present study were able to predict progression and/or survival to a more limited extent than α2-6 sialylation. The prognostic capacity of β1-4 and β1-6 branching however, may, at least in part, be due to the fact that these glycan features simply create greater opportunity for sialylation. Beyond this study, others have found that the sialyl Lewis X epitope (which displays α2-3 sialylation rather than α2-6 sialylation) predicts progression and survival in both small cell⁶⁹ and non-small cell lung cancer.⁷⁰⁻⁷² Like the prognostic Veristrat markers,⁷³⁻⁷⁵ which are serum amyloid A proteoforms⁷⁶, elevated α2-6 sialylation in lung cancer may largely be due to an inflammatory response by the liver. But if, as described above, sialylation-based cloaking of tumor cells from the immune system plays an important role in the metastatic process, α2-6 sialylation may turn out to play a causative, mechanistic role in lung cancer progression.

### Conclusions

A molecularly bottom-up approach to plasma/serum (P/S) glycomics based on glycan linkage analysis that captures unique glycan features such as α2-6 sialylation, β1-6 branching and core fucosylation as single analytical signals was employed to evaluate the behavior of P/S glycans in all stages of lung cancer and across various stages of prostate, ovarian and pancreatic cancers. Elevation of terminal (total) fucosylation, α2-6 sialylation, β1-4 branching, β1-6 branching and outer-arm fucosylation markers were most pronounced in lung cancer in a stage-dependent manner, but these changes were found to be independent of the tumor tissue-of-origin. Using a Cox proportional hazards regression model, the marker for α2-6 sialylation was found to predict both progression and all-cause mortality in lung cancer patients after adjusting for age, gender, smoking status and stage at which the sample was taken. Interestingly, certifiably healthy P/S donors had markedly lower levels of α2-6 sialylation, β1-4 branching and β1-6 branching relative to cancer risk-matched controls. While early detection is ideal, the information provided by this and related studies^{31,33-35, 41,42,49-53} suggests that pre-cancerous inflammation may be responsible for the elevation of many of the glycan features observed in the at-risk controls relative to the certifiably healthy donors—implying that the goal of preventing such a pre-cancerous state may be as important as preventing the transition from an at-risk state to stage I cancer.

### Example 2

Our recent large lung cancer study provided important information about the diagnostic and prognostic value of P/S glycan nodes in lung cancer as well as other types of cancer.²¹ In particular, we observed strong stage-dependence, but tissue-of-tumor-origin independence of elevated P/S glycan features. Moreover, we found that glycan nodes corresponding to α2-6 sialylation, β1-4 branching and β1-6 branching were able to predict survival and progression. The primary purposes of this study were to evaluate the ability of unique glycan features, quantified via glycan node analysis, to 1) evaluate the potential ability of glycan nodes to distinguish MIBC from NMIBC, 2) distinguish NMIBC patients from patients with a history of bladder cancer but currently exhibiting no clinical evidence of disease (NED), and 3) evaluate the ability of glycan nodes to predict recurrence from a state of remission (i.e., the NED state). Based on our observations in lung cancer²¹, we anticipated findings of potential clinical interest under each objective. Moreover, elevated blood plasma protein glycosylation is known to be associated with inflammation in some non-cancerous clinical conditions.²²⁻²⁴ Since C-reactive protein (CRP) is a well-studied marker of inflammation²⁵ as well as a prognostic marker for UCC²⁶⁻²⁹, we also evaluated the quantitative relationship between glycan nodes that were prognostically useful in NED patients and CRP.

The primary purposes of Example 2 were to evaluate the ability of unique glycan features, quantified via glycan node analysis, to 1) evaluate the potential ability of glycan nodes to distinguish MIBC from NMIBC, 2) distinguish NMIBC patients from patients with a history of bladder cancer but currently exhibiting no clinical evidence of disease (NED), and 3) evaluate the ability of glycan nodes to predict recurrence from a state of remission (i.e., the NED state). Based on our observations in lung cancer in Example 1, we anticipated findings of potential clinical interest under each objective. Moreover, elevated blood plasma protein glycosylation is known to be associated with inflammation in some non-cancerous clinical conditions [22-24]. Since C-reactive protein (CRP) is a well-studied marker of inflammation [25] as well as a prognostic marker for UCC [26-29], we also evaluated the quantitative relationship between glycan nodesthat were prognostically useful in NED patients and CRP.

### Materials and Methods

Plasma Samples EDTA plasma samples from MIBC (n=12), NMIBC (n=39) and NED patients (n=72), as well as certifiably healthy living kidney donors (n=30) were enrolled in the Multidisciplinary Biobank at Mayo Clinic Arizona under a Mayo Clinic Institutional Review Board (IRB)-approved protocol. Patients eligible for enrollment were those seen at Mayo Clinic Arizona who were >18 years old, able to provide informed consent, and undergoing evaluation as either a potential living kidney donor or for genitourinary diseases. Detailed inclusion & exclusion criteria for living kidney donors are provided in Supporting Information (S1 Appendix). None of the living kidney donor patients smoked at the time of health screening and blood collection; 27% were former smokers and 73% never smoked. Living kidney donor and UCC patients were excluded if they declined to participate or if the banking of their biospecimens would compromise the availability of tissue for diagnosis and standard clinical care. All specimens were collected during the time frame of June 2010 through February 2016. Standard operating protocols and blood collections were performed as previously described [30]. All specimens were stored at −80° C. prior to shipment to ASU and maintained at −80° C. at ASU prior to analysis. All specimens were analyzed blind and in random order. An aliquot of plasma from the same individual donor was analyzed in every batch as a quality control (QC) specimen to ensure batch-to-batch consistency.

This research was approved by Arizona State University's IRB and all clinical investigations were conducted according to the principles expressed in the Declaration of Helsinki.

### Glycan Node Analysis

Sample Preparation Glycan node analysis was performed on the plasma samples as described previously¹⁹. Briefly, it includes four main steps (FIG. 2): 1) permethylation, in which 9 μL of plasma sample containing 1 g L of a 10 mM solution of heavy-labeled D-glucose (U-¹³C₆, 99%; 1,2,3,4,5,6,6-D₇, 97%-98%) (Cambridge Isotope Laboratories), and N-acetyl-D-[UL-¹³C₆]glucosamine (Omicron Biochemicals, Inc.) as the internal standard was mixed with 270 μL of dimethylsulfoxide (DMSO) (Sigma-Aldrich) followed by 105 μL of iodomethane (99%, Cat. No. 18507, Sigma-Aldrich). Then, this mixture was added to a plugged 1-mL spin column (ThermoFisher Scientific, Waltham, Mass., Cat. No. 69705) containing ˜0.7 g of sodium hydroxide beads (20-40 mesh, Sigma-Aldrich) which had been preconditioned by acetonitrile (Fisher Scientific) and washed twice with DMSO prior to addition of sample. After occasionally stirring the NaOH column over 11 min, the unplugged samples were spun for 15 s at 5,000 rpm (2,400 g) in a microcentrifuge to extract the permethylated glycans. In order to maximize glycan recovery, 300RL of acetonitrile was added to the NaOH column and spun down for 30 s at 10,000 rpm (9,600 g). Then, in a silanized 13×100 glass test tube holding 3.5 mL of 0.2 M sodium phosphate buffer, the solution from the first spin-through was added and mixed well. After pooling and mixing the second acetonitrile-based spin-through solution was combined with the rest of the sample, followed by 1.2 mL of chloroform (Sigma-Aldrich). The test tube was then capped and shaken well, followed by removal and discard of the aqueous layer. After two additional rounds of liquid/liquid extraction, the chloroform layer was recovered and dried under nitrogen at 74° C. 2) The second major step was TFA hydrolysis, in which 325 μL of 2M trifluoroacetic acid (TFA) (Sigma-Aldrich) was added to each test tube. After capping the samples and incubating them at 121° C. for 2 h, they were dried down under nitrogen at 74° C. 3) The third major step involved reduction of sugar aldehydes, in which the samples were incubated for an hour after adding 475 μL of a freshly made 10 mg/mL solution of sodium borohydride (Sigma-Aldrich) in 1M ammonium hydroxide (Sigma-Aldrich). Then 63 μL of methanol (Honeywell Burdick & Jackson) was added to each sample before drying at 74° C. under nitrogen. Next, 125 μL of a 9:1 (v/v) methanol: acetic acid solution was added to each test tube followed by drying under nitrogen. To fully dry the samples, they were then placed in vacuum desiccator for approximately 20 min. 4) The fourth major step consisted of acetylation of nascent hydroxyl groups, in which the sample residue in each test tube was dissolved by 18 μL water before adding 250 μL of acetic anhydride (Sigma-Aldrich). After sonicating the samples for 2 min and incubating for 10 min at 60° C., 230 μL of concentrated TFA was added to each sample, followed by incubation of the capped samples for 10 min at 60° C. Then, 2 mL methylene chloride (Fisher Scientific) was added to each sample followed by 2 mL of water. Next, liquid/liquid extraction was done twice in which the methylene chloride layer was saved and then transferred into a silanized autosampler (ThermoFisher Scientific), dried under nitrogen, reconstituted in 120 μL of acetone (Avantor Performance Materials), and capped to be injected onto the GC-MS.

Gas Chromatography-Mass Spectrometry As previously described,²¹ an Agilent Model A7890 gas chromatograph (equipped with a CTC PAL autosampler) was used coupled to a Waters GCT (time-of-flight) mass spectrometer to analyze the prepared samples. For all samples, one injection of 1 μL was made at split ratio of 20:1 onto an Agilent split-mode liner containing a small plug of silanized glass wool with the temperature set to 280° C. The DB-5 ms GC column that was used for chromatography was 30 m. The oven temperature, initially kept at 165° C., was increased at a rate of 10° C./min up to 265° C. Immediately after that, the temperature was increased at a rate of 30° C./min to 325° C., then held constant for 3 min. The transfer line to the mass spectrometer was kept at 250° C. Following the elution of sample components from the GC column, they were subjected to electron ionization (70 eV, 250° C.) and analyzed in the m/z range of 40-800 with a scan cycle time of 0.1 s. Daily calibration and tuning of the mass spectrometer was done using perfluorotributylamine.

The quantification method is described in detail elsewhere.¹⁸ Briefly, summed extracted ion chromatogram peaks were integrated automatically and checked manually using QuanLynx software. The collected data were then exported to a spreadsheet for detailed analysis.

Human C-Reactive Protein ELISA Assay The Invitrogen™ Human C-Reactive Protein ELISA kit (Catalog Number KHA0031, ThermoFisher Scientific) was used, following the manufacturer instructions, to measure the concentration of CRP in patient plasma samples. Final absorbance values were read at 450 nm by Thermo Scientific Multiskan Go plate reader and the concentration of samples were calculated using SkanIt Software 3.2.

Statistical Analysis Individual extracted-ion chromatographic peak areas for each glycan node were normalized using one of two possible approaches: 1) Individual hexose residues were normalized to heavy glucose and individual N-acetylhexosamine (HexNAc) residues were normalized to heavy N-acetyl glucosamine (heavy GlcNAc). 2) Individual hexose residues were normalized to the sum of all endogenous hexose residues. Likewise, each HexNAc residue was normalized to the sum of all endogenous HexNAcs. The average % CV calculated based on the analysis of the QC sample in each batch shows that the latter normalization method provides better inter-batch reproducibility (<10% for the four most elevated glycan nodes) but the former normalization method performs better in separating the patient groups while still keeping the average inter-batch % CV in an acceptable range (i.e., <18%). Unless otherwise noted, results described below are based on normalization with heavy glucose and heavy GlcNAc. All extracted-ion chromatographic peak areas for all samples, including their normalization to heavy glucose or heavy GlcNAc and normalization to the sum of all endogenous hexoses or HexNAcs as well as % CV values for batch-to-batch QC samples are included in Supporting Information accompanying Ferdosi S et al., PloS One 2018, 13(7) (S1 File).

For both the glycan node data and the CRP ELISA data, outliers within each clinical group (Control, NED, NMIBC and MIBC) were removed after log10 transformation using the ROUT method at Q=1% by GraphPad Prism 7. After removing the outliers, the anti-log of each value was taken to reverse the transformation. To identify differences between clinical groups, the Kruskal-Wallis test was performed followed by the Benjamini-Hochberg false discovery correction procedure at a 5% false discovery rate using RStudio Version 1.0.143. Univariate distributions and ROC curves were plotted using GraphPad Prism 7. The ability of certain glycan nodes to predict bladder cancer recurrence was evaluated by performing Cox proportional hazards regression models using SAS 9.4. Correlations between CRP and glycan nodes were examined using Pearson correlation in GraphPad Prism 7.

### Results

Altered Glycan Features in UCC The relative abundance of 19 glycan “nodes” was quantified in each of the control, NED, NMIBC, and MIBC patient samples. Each of these nodes contributed at least 1% of the sum total of all hexoses or all HexNAcs. Data normalized to heavy, stable isotope-labeled glucose and GlcNAc internal standards were first evaluated for statistically significant differences between all four patient groups. No differences were found between MIBC, NMIBC and NED patients (Table 1). However, relative to the certifiably healthy controls, statistically significant changes were found in more than half of the glycan nodes measured in NED, NMIBC, and MIBC patients (Table 1). Among these glycan nodes, the only one that was decreased in the current and former cancer patient samples was 4-linked glucose (i.e., 4-Glc, which is mostly derived from glycolipids). The same trend was previously observed in lung cancer patient samples.²¹ The rest of the altered nodes were increased in current and former UCC patients compared to the certifiably healthy controls.

TABLE 1
Statistically significant differences between controls
and bladder cancer patient sub-cohorts.^{a}

| Glycan | Control | Control | Control | NED vs. | NED vs. | NMIBC |
|---|---|---|---|---|---|---|
| Nodes ^{b, c} | vs. NED | vs. NMIBC | vs. MIBC | NMIBC | MIBC | vs MIBC |
| t-Fucose | i | i | ii | ns | ns | ns |
| t-Gal | ns | ns | ns | ns | ns | ns |
| 2-Man | iii | ii | iii | ns | ns | ns |
| 4-Glc | ns | d | ns | ns | ns | ns |
| 3-Gal | ns | ns | ns | ns | ns | ns |
| 6-Gal | iiii | iii | ii | ns | ns | ns |
| 3,4-Gal | ns | ns | ns | ns | ns | ns |
| 2,4-Man | iii | ii | i | ns | ns | ns |
| 2,6-Man | iiii | iiii | ii | ns | ns | ns |
| 3,6-Man | i | ns | ns | ns | ns | ns |
| 3,6-Gal | ns | ns | ns | ns | ns | ns |
| 3,4,6-Man | i | i | i | ns | ns | ns |
| t-GlcNAc | i | i | ns | ns | ns | ns |
| 4-GlcNAc | ii | ii | i | ns | ns | ns |
| 3-GlcNAc | ii | i | ii | ns | ns | ns |
| 3-GalNAc | ns | ns | i | ns | ns | ns |
| 3,4-GlcNAc | ii | ii | ii | ns | ns | ns |
| 4,6-GlcNAc | ns | ns | ns | ns | ns | ns |
| 3,6-GalNAc | iii | ii | iii | ns | ns | ns |

^{a}Individual hexose residues were normalized to heavy glucose and individual HexNAc residues were normalized to heavy GlcNAc).
^{b} Significance was determined by the Kruskal-Wallis test followed by the Benjamini-Hochberg correction procedure at a 5% false discovery rate.
^{c} “ns” stands for “not significant”. “i” and “d” stand for “increased” or “decreased” glycan levels in the cohort with clinically more advanced disease listed in the column header. “i” or “d” indicates p < 0.05, “ii” or “dd” indicates p < 0.01, “iii” or “ddd” indicates p < 0.001, and “iiii” or “dddd” indicates p < 0.0001.

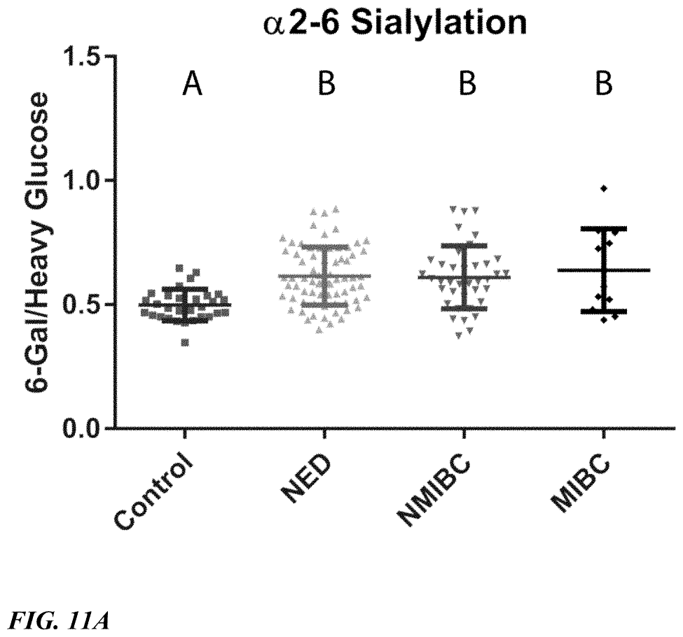

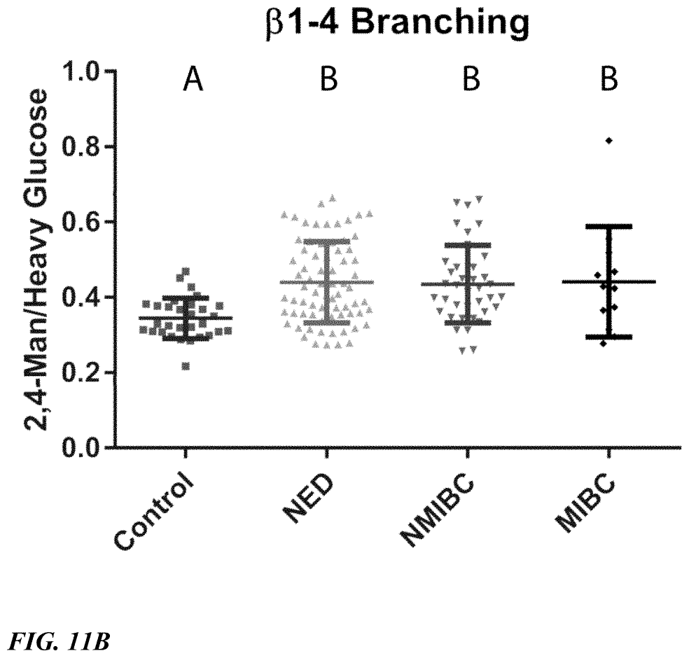

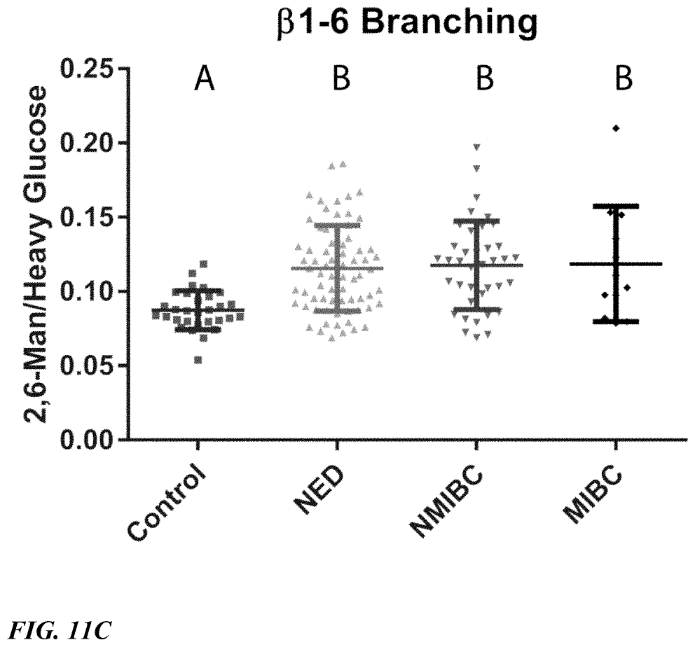

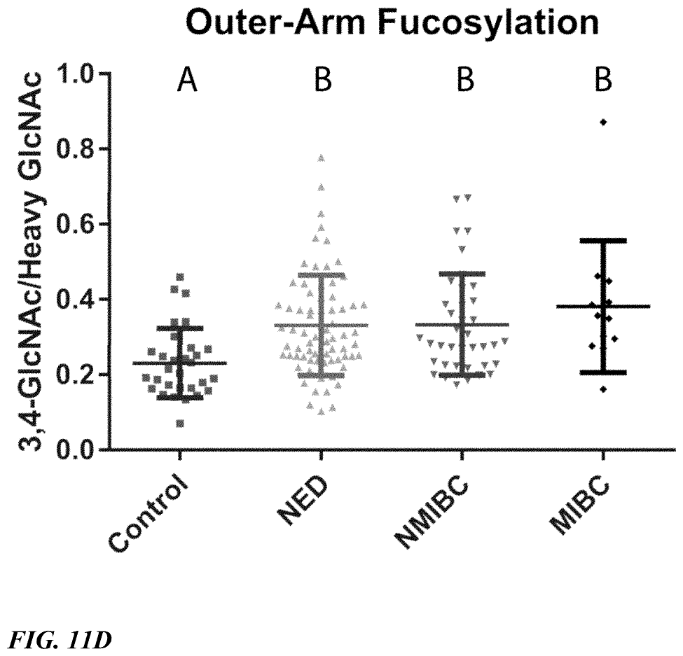

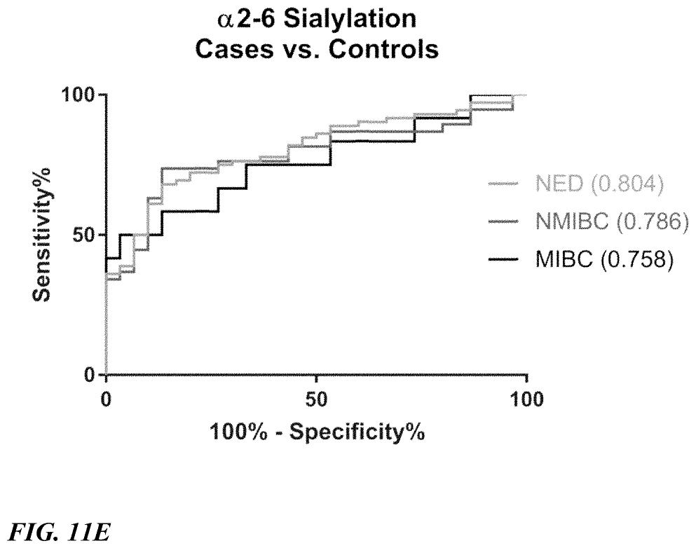

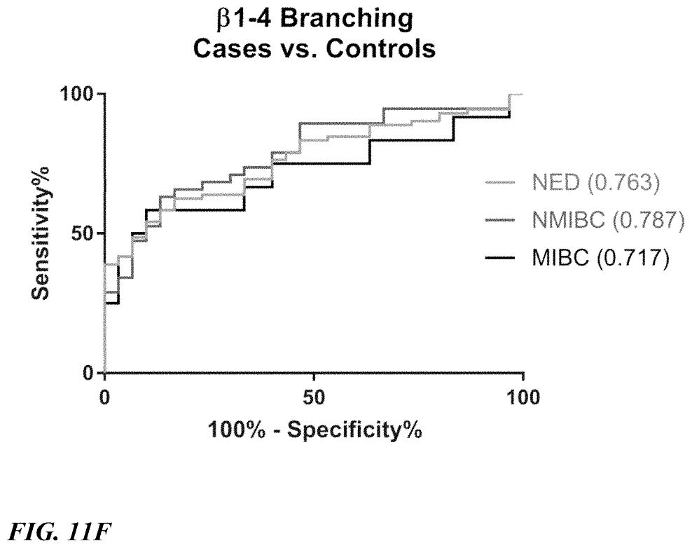

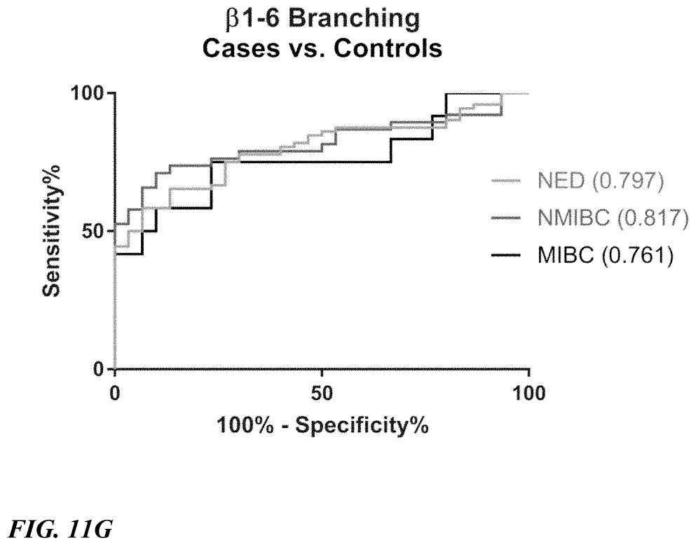

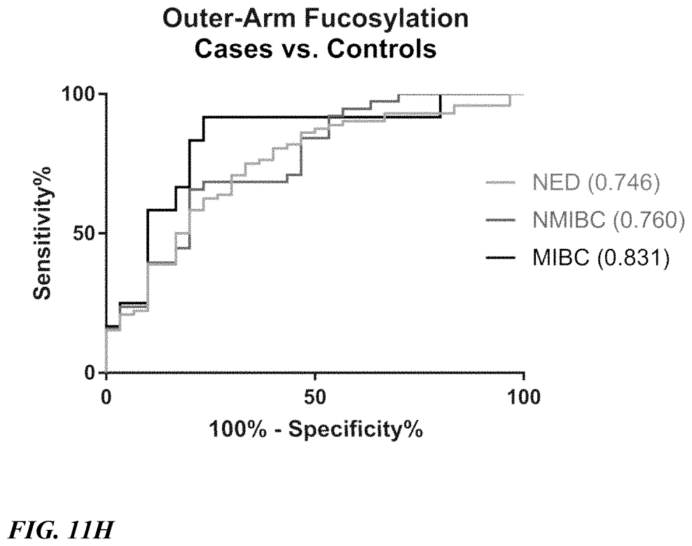

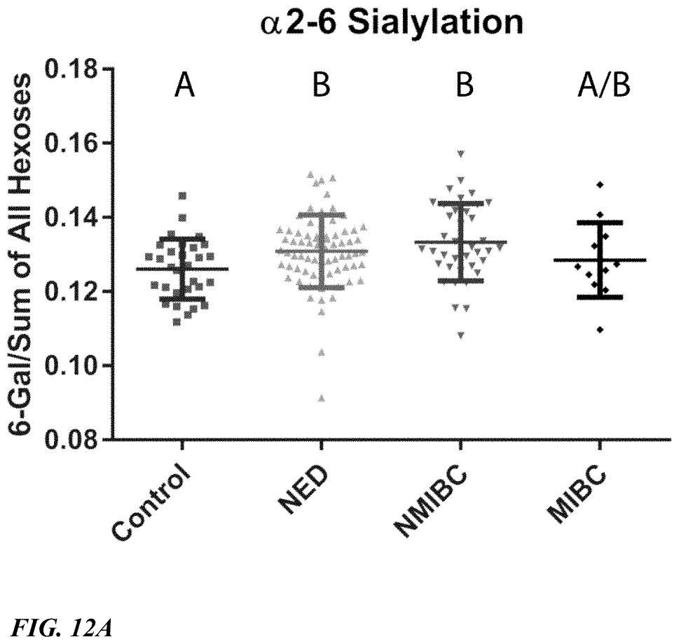

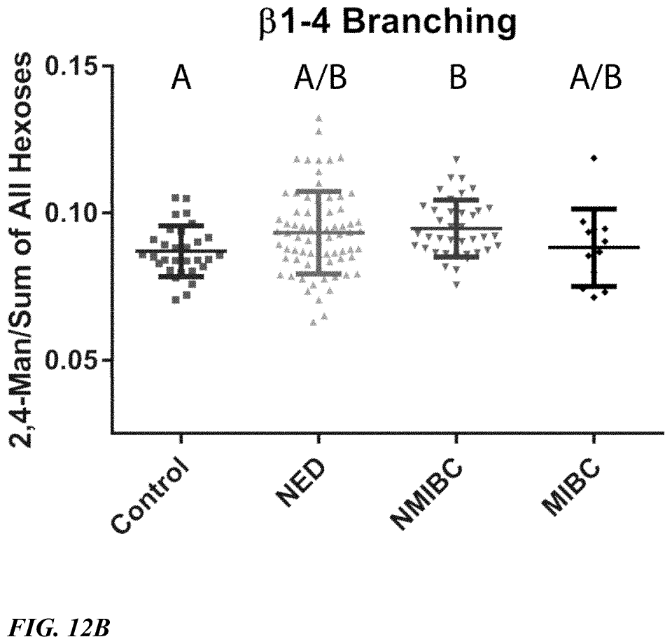

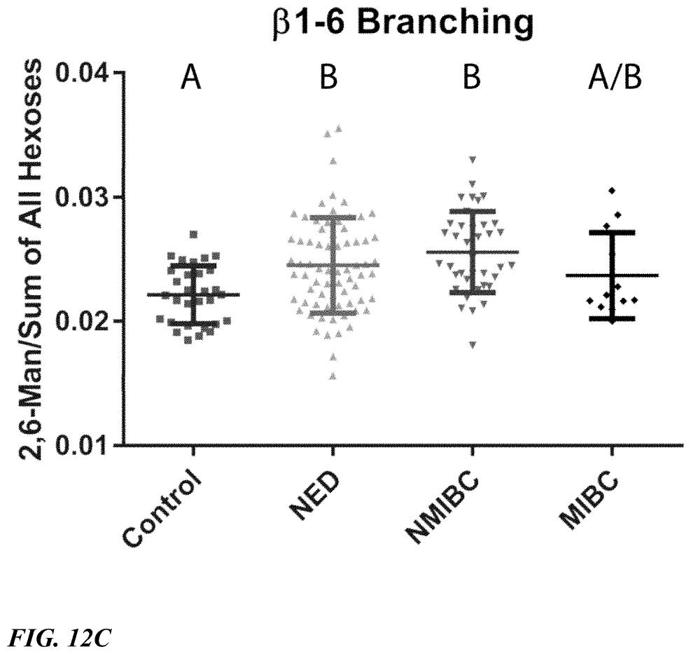

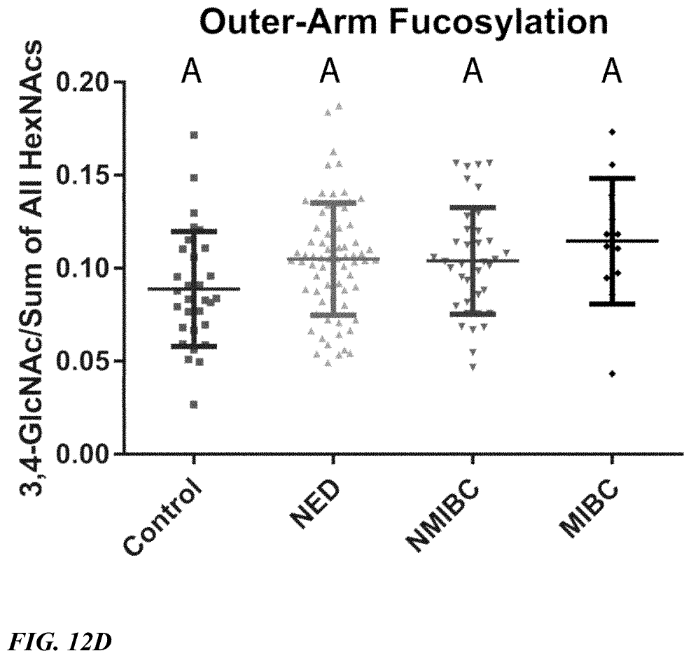

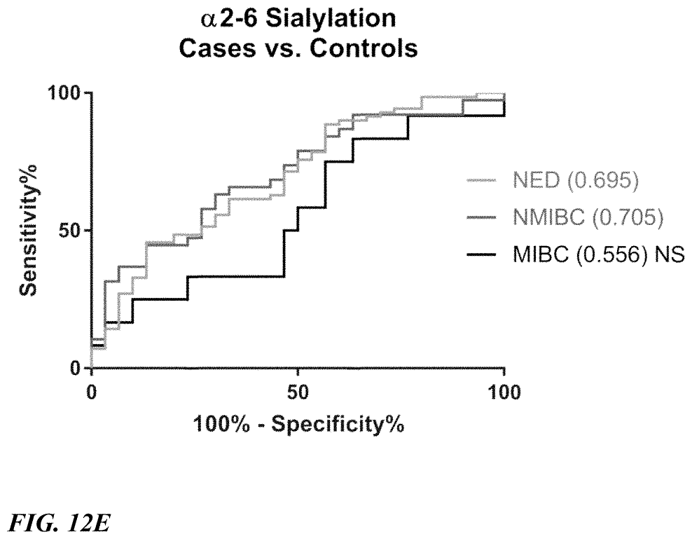

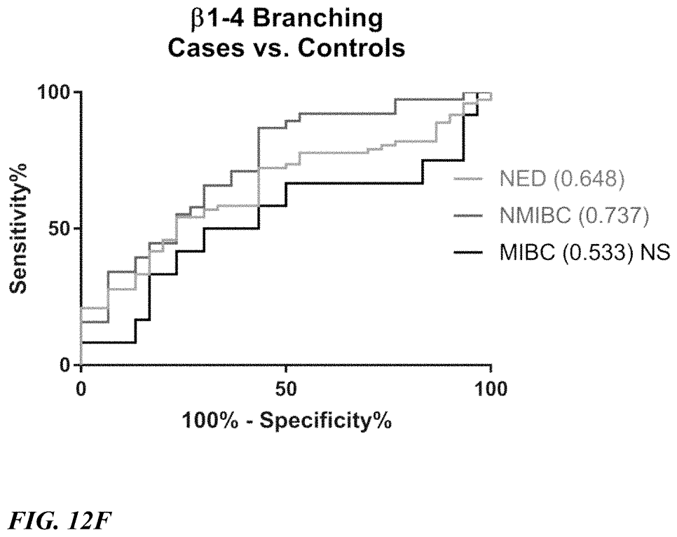

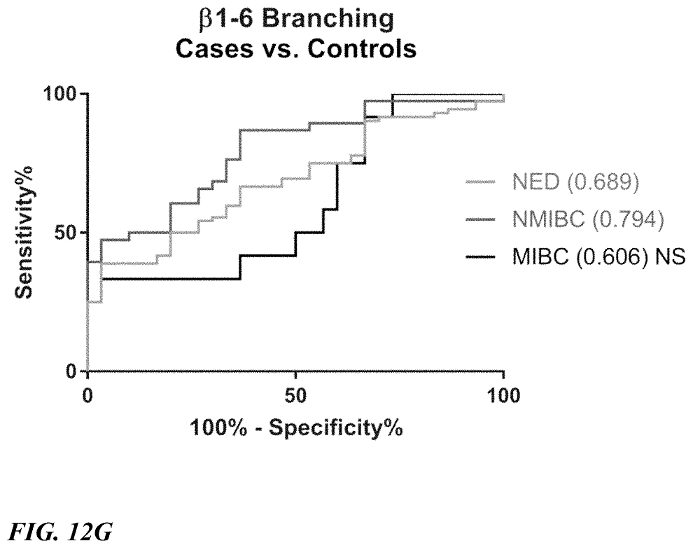

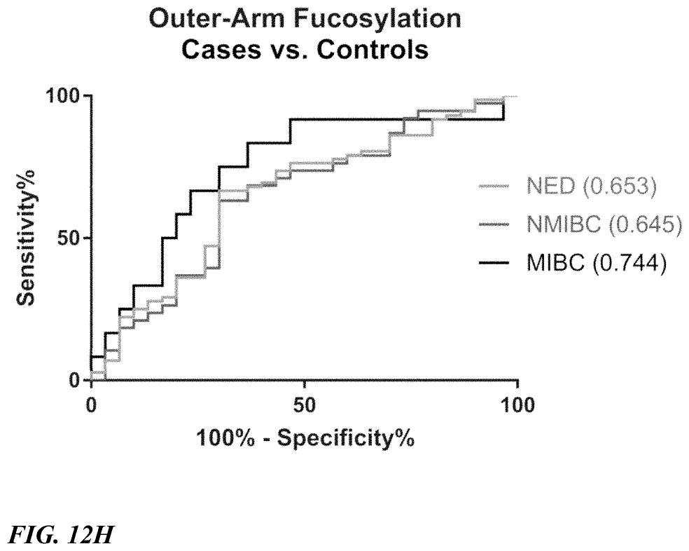

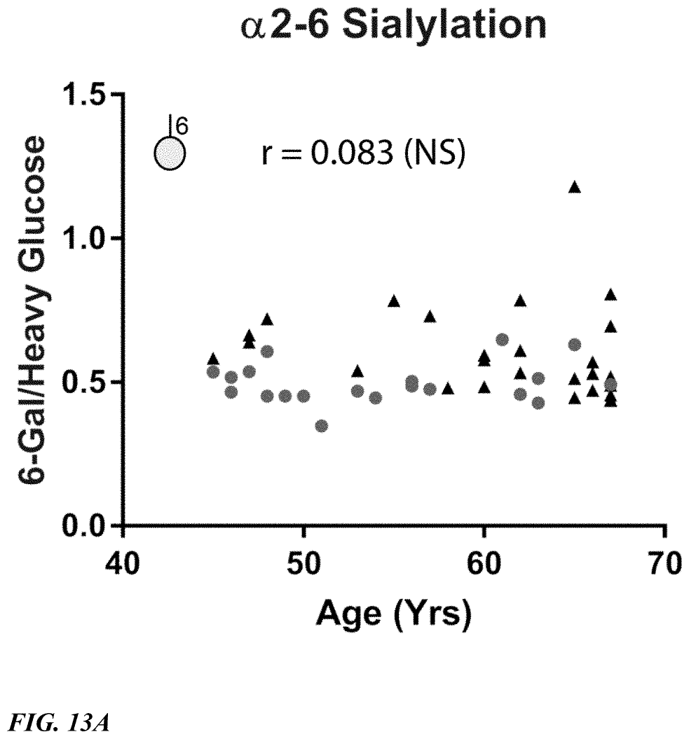

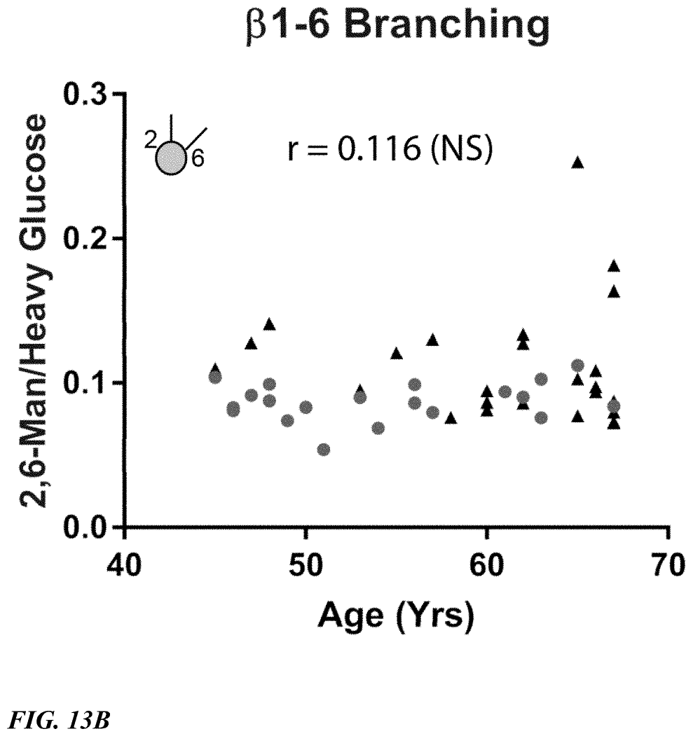

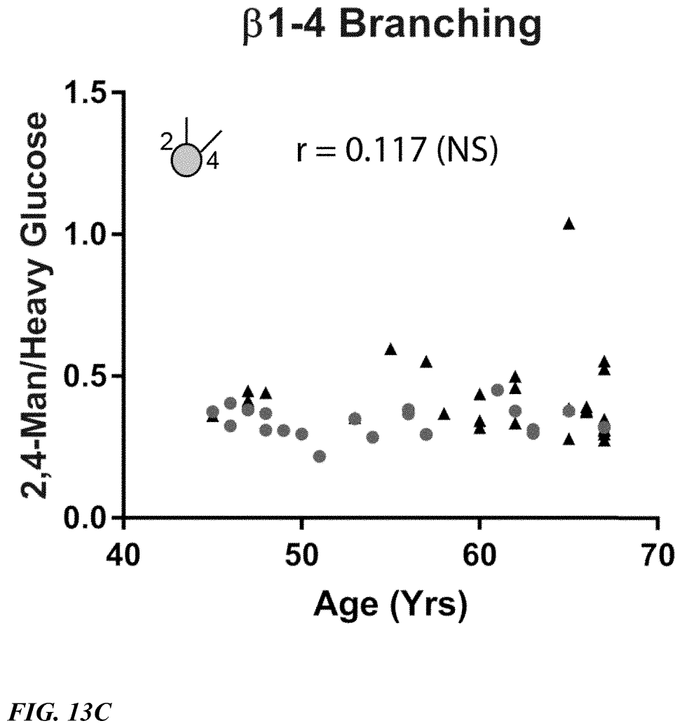

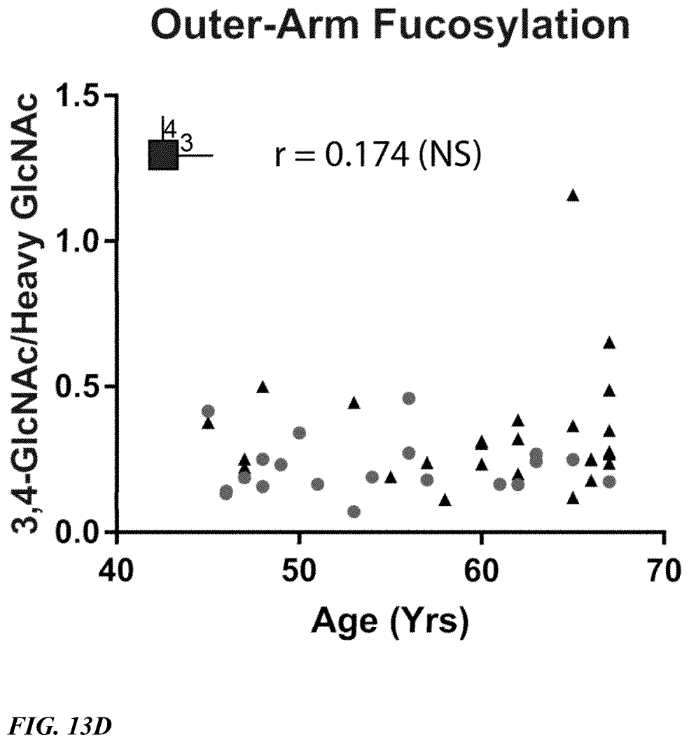

There were four glycan nodes that were most elevated in the current and former UCC patients relative to the certifiably healthy controls, including 6-linked galactose, 2,4-linked mannose, 2,6-linked mannose, and 3,4-linked GlcNAc. These nodes correspond to α2-6 sialylation, β1-4 branching, β1-6 branching, and outer-arm fucosylation, respectively.^{18,21} The univariate distributions of these four glycan nodes in each of the four clinical groups are shown in FIG. 11A-FIG. 11H. ROC curves for current and former UCC patients vs. the certifiably healthy controls are also shown (FIG. 11A-FIG. 11H). The distribution of each of these glycan nodes within each cohort shows that patients with no evidence of disease (NED) have similar glycosylation profiles to patients with active disease (NMIBC and MIBC) and that significant increases in these glycan nodes can only be seen when comparing current and former UCC patients to the certifiably healthy controls—but not when comparing amongst the three current and former UCC patient subgroups (Table 1 and FIG. 11A-FIG. 11H). Data normalized to the sum of endogenous hexoses or HexNAcs were not as effective at distinguishing the control specimens from those from current or former bladder cancer patients (Table 2 and FIG. 12A-FIG. 12H). However, as explained in the Discussion section, these data indicate that significant qualitative shifts in glycan composition are observed in current and former UCC patients as opposed to mere increases in the absolute abundance of glycans.

TABLE 2
Statistically significant differences between controls
and bladder cancer patient sub-cohorts with data normalization
to the sum of all endogenous hexoses or HexNAcs.

| Glycan | Control | Control | Control | NED vs. | NED vs. | NMIBC |
|---|---|---|---|---|---|---|
| Nodes ^{a, b} | vs. NED | vs. NMIBC | vs. MIBC | NMIBC | MIBC | vs MIBC |
| t-Fucose | ns | ns | ns | ns | ns | ns |
| t-Gal | dd | dd | ns | ns | ns | ns |
| 2-Man | ns | ns | ns | ns | ns | ns |
| 4-Glc | ddd | dddd | d | ns | ns | ns |
| 3-Gal | ns | ns | ns | ns | ns | ns |
| 6-Gal | i | ii | ns | ns | ns | ns |
| 3,4-Gal | ns | ns | ns | ns | ns | ns |
| 2,4-Man | ns | ns | ns | ns | ns | ns |
| 2,6-Man | ii | iii | ns | ns | ns | ns |
| 3,6-Man | ns | ns | ns | ns | ns | ns |
| 3,6-Gal | ns | ns | ns | ns | ns | ns |
| 3,4,6-Man | ns | ns | ns | ns | ns | ns |
| t-GlcNAc | ns | ns | ns | ns | ns | ns |
| 4-GlcNAc | ns | ns | ns | ns | ns | ns |
| 3-GlcNAc | ns | ns | ns | ns | ns | ns |
| 3-GalNAc | dd | d | ns | ns | ns | ns |
| 3,4-GlcNAc | i | ns | i | ns | ns | ns |
| 4,6-GlcNAc | ns | dd | ns | ns | ns | ns |
| 3,6-GalNAc | ns | ns | ns | ns | ns | ns |

^{a} Significance was determined by the Kruskal-Wallis test followed by the Benjamini-Hochberg correction procedure at a 5% false discovery rate.
^{b} “ns” stands for “not significant”. “i” and “d” stand for “increased” or “decreased” glycan levels in the cohort with clinically more advanced disease listed in the column header. “i” or “d” indicates p < 0.05, “ii” or “dd” indicates p < 0.01, “iii” or “ddd” indicates p < 0.001, and “iiii” or “dddd” indicates p < 0.0001.

The average age of the certifiably healthy living kidney donors (controls) was 47, while the average age for the NED, NMIBC and MIBC patients was 74, 76, and 73, respectively. Yet after correcting for multiple comparisons, no statistically significant correlation of any glycan node with age could be found when pooling data from all cohorts and evaluating correlations for the age range in which there was overlap between the controls and the current and former UCC patients (i.e., ages 45-67; see FIG. 5A-FIG. 5O). Likewise, no significant correlations with age were observed within the certifiably healthy controls or within the current/former UCC patients when these groups were considered in isolation (not shown).

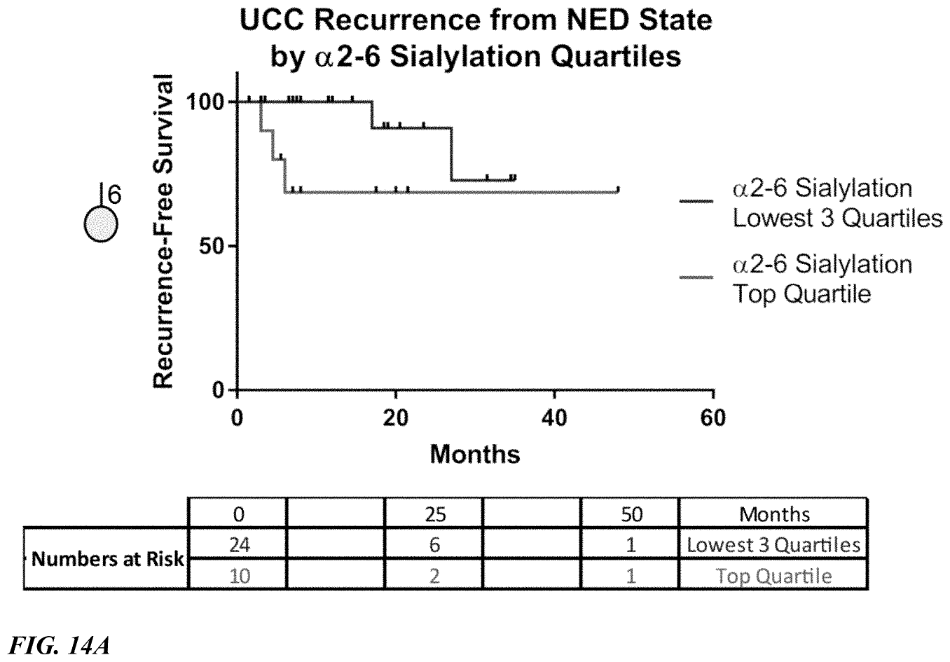

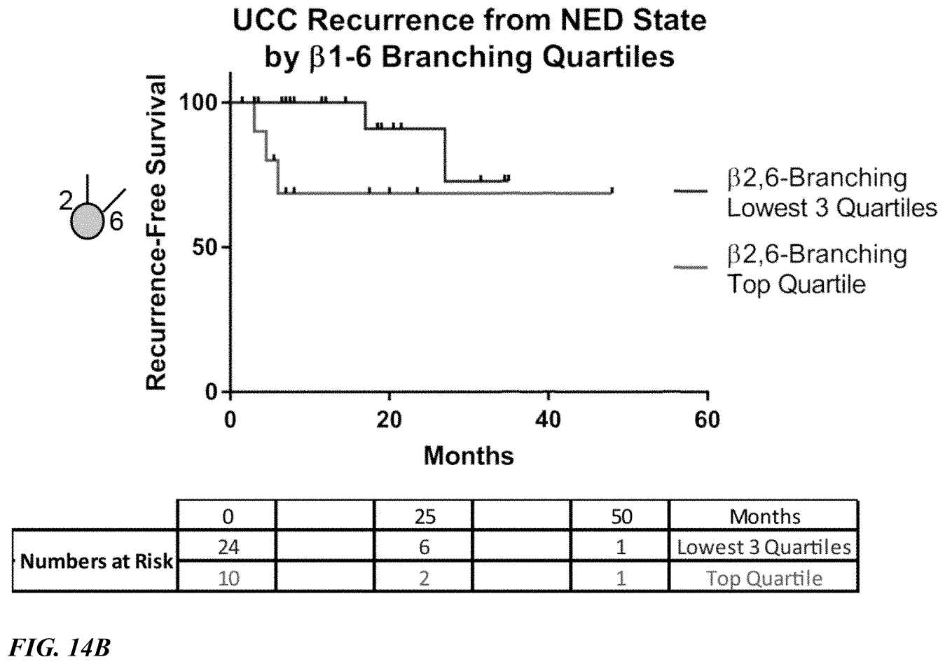

Prognostic Value of Glycan Nodes Within the NED cohort there were numerous samples with high levels of specific glycan nodes that were well out of the range observed in the controls—and which were similar to the cancer patient samples—even though the NED patients were clinically free of disease (FIG. 11A-FIG. 11H). These observations led to evaluation of the ability of glycan nodes to predict recurrence in a Cox proportional hazards regression model. After breaking down glycan node data into quartiles and adjusting for age, gender, and time from cancer (i.e., time elapsed since there was evidence of cancer in a NED patient), 6-linked galactose and 2,6-linked mannose, which correspond to α2-6 sialylation and β1-6 branching, respectively, predicted recurrence with p-values of <0.05. The top α2-6 sialylation quartile predicted recurrence from the NED state with a hazard ratio of 15 relative to all other quartiles combined (lower bound at 95% CL=1.3; upper bound at 95% CL=180; p=0.029). Similarly, the top β1-6 branching quartile predicted recurrence from the NED state with a hazard ratio of 11 relative to all other quartiles combined (lower bound at 95% CL=1.2; upper bound at 95% CL=110; p=0.037). The differences in the rates of recurrence for the top α2-6 sialylation and β1-6 branching quartiles compared to all other quartiles are shown in the progression-free survival curves (FIG. 14A-FIG. 14B).

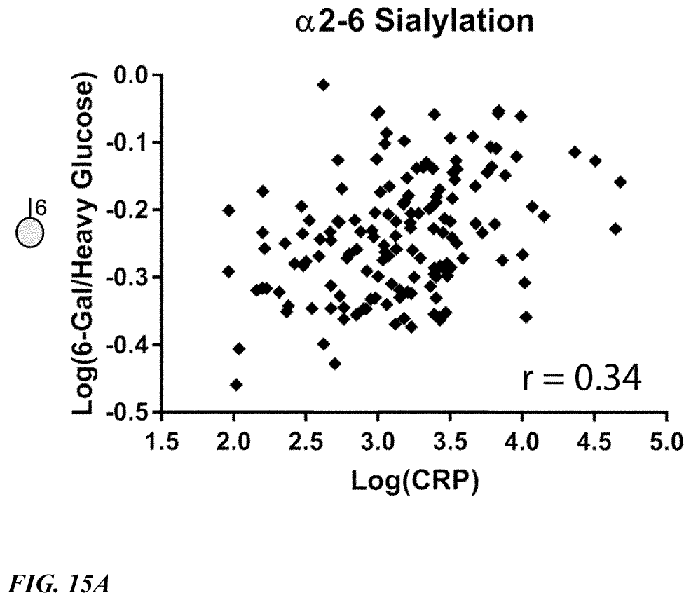

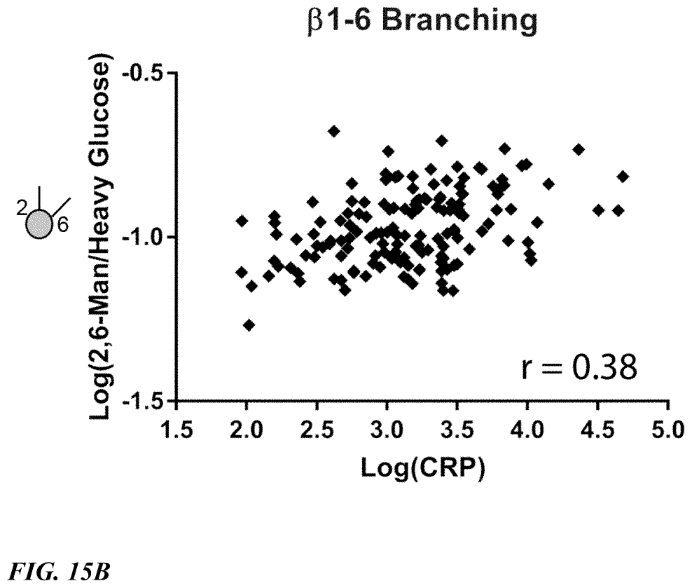

CRP Correlation with Glycan Nodes CRP was measured in order to correlate changes in patient glycan nodes with patient inflammation status. The average level of CRP in the certifiably healthy controls was 1.76 mg/L whereas the NED, NMIBC, and MIBC samples had average CRP levels of 3.84, 3.21, and 3.08 mg/L, respectively (which are above the normal range of CRP (<3.0 mg/L) [28]). The levels of 6-linked galactose, which corresponds to α2-6 sialylation, positively correlated with CRP (r=0.34, p<0.001), as did the levels of 2,6-linked mannose, which corresponds to β1-6 branching (r=0.38, p<0.001) (FIG. 15A-FIG. 15B).

### Discussion

Out of 19 quantified glycan nodes, four of them, each corresponding to a unique glycan feature including α2-6 sialylation, β1-4 branching, β1-6 branching and outer-arm fucosylation, were most significantly elevated in UCC patients compared to certifiably healthy individuals (Table 1 and FIG. 11A-FIG. 11H). Unexpectedly, cancer-free patients with a history of UCC (NEDs) had glycan node distributions that were similar to both the early and late-stage cancer patients but distinct from the controls (FIG. 11A-FIG. 11H). And, unlike other types of cancers that we have reported upon previously²¹, glycan node-based features were at the same level in later stages of UCC (MIBC patients) as in earlier stages (NMIBC patients). These findings were unanticipated and indicate that the distinct plasma glycan features such as α2-6 sialylation, β1-4 branching, β1-6 branching, bisecting GlcNAc and core fucosylation that were directly quantified by glycan node analysis are not capable of distinguishing patients with active UCC from patients in remission.

In order to interpret the physiological significance of these findings, it must be understood that the glycans being measured are from high-concentration glycoproteins derived primarily from the liver (i.e., transferrin, alpha-2-macroglobulin, haptoglobin, etc) and the immune system (i.e., IgG antibody glycans) rather than being sloughed off or secreted by cancer cells themselves^{31,32}. These macro-level (mg/mL scale) changes in blood plasma glycan biochemistry are thought to be mediated, at least in part, by cytokines secreted from the tumor which are recognized by the liver and/or immune system as part of a systemic inflammatory response, altering the way that these two major glycoprotein-producing systems glycosylate their proteins.³³⁻³⁸

With this in mind, there are three possible causes for the increases in various glycan nodes observed in Table 1 and FIG. 11A-FIG. 11H. First, the acute phase response in current and former UCC patients (evidenced by elevated CRP) may induce a net increase in the total concentration of plasma glycoproteins—and more glycoproteins means more glycans. Second, glycoprotein site occupancy may increase. While this possibility has not been extensively studied, some evidence exists that subtle but statistically significant increases in site occupancy may occur in steatosis and non-alcoholic steatohepatitis.³⁹ Third, the qualitative nature of the glycans themselves may change. This phenomenon has repeatedly been documented in cancer and is often the primary reason for shifts in glycan profiles—particularly when the data reported are compositional in nature (i.e., all signals sum to 100%).^{23,40,41} When the glycan node data from this study are normalized to the sum of endogenous hexose residues or HexNAc residues, statistically significant increases in the four most elevated glycan nodes are observed in current and former UCC patients relative to the healthy controls (FIG. 12A-FIG. 12H)—though these increases tend not to be as strong as when total glycan node quantities are considered (FIG. 11A-FIG. 11H)—i.e., when the data are normalized to heavy labeled internal standards. Altogether, elevated CRP levels and the data seen in FIG. 12A-FIG. 12H suggest that both the first and the third possible explanations likely contribute to our observations. Assessing changes in glycan site occupancy requires establishing a complex, custom assay for each protein in question and is beyond the scope of the present study.

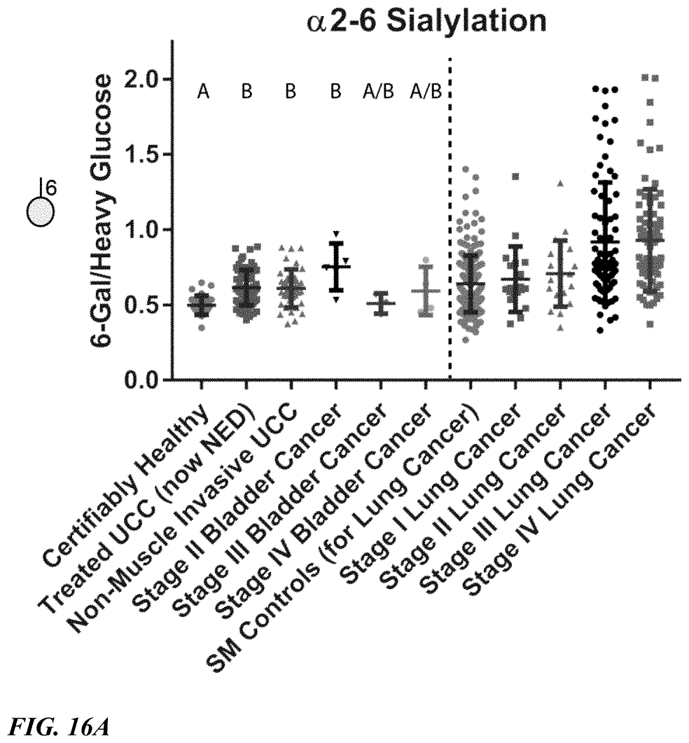

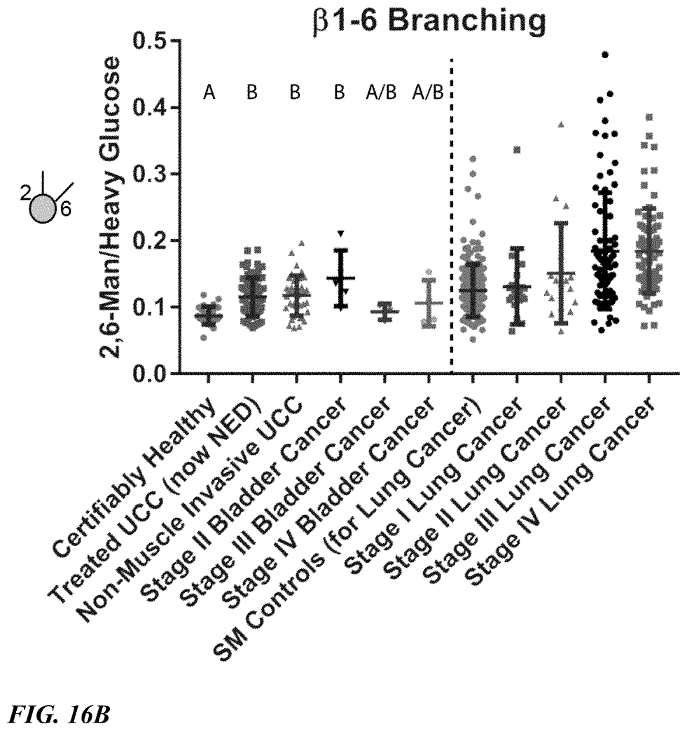

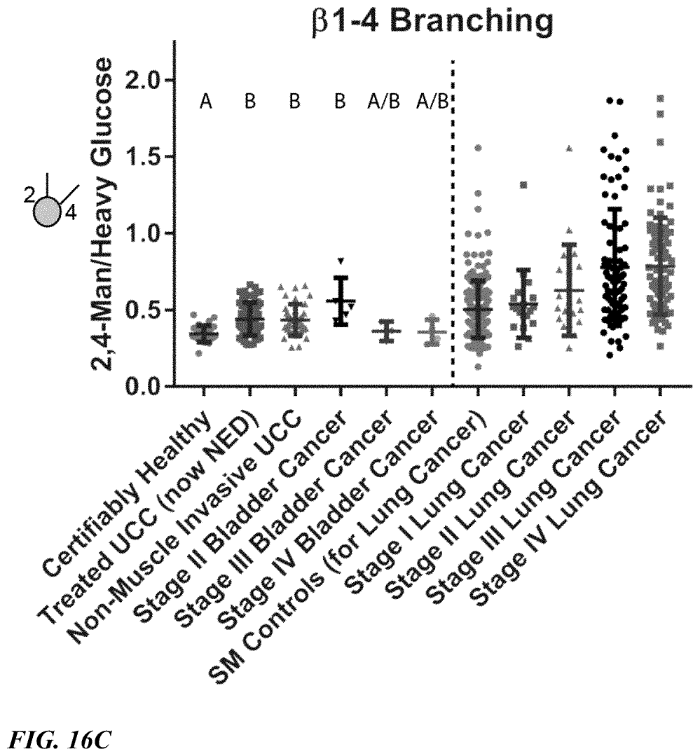

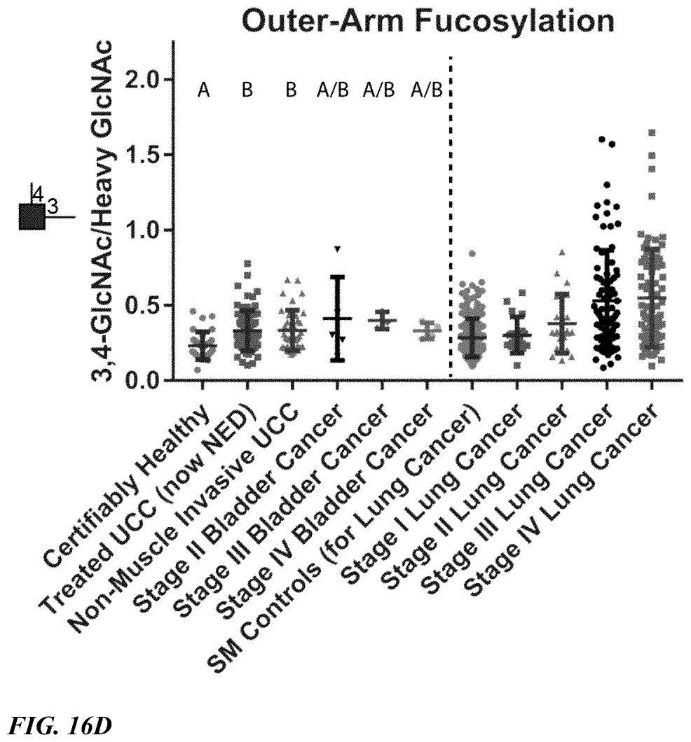

Overall, the glycan node distributions observed here in UCC suggest that UCC makes modest, early-stage alterations to blood plasma glycans that, even at stages III-IV, do not reach the extreme levels observed in pancreatic, ovarian, lung and other types of cancer.²¹ To illustrate, lung cancer patient glycan node data from Example 1 are compared side-by-side with UCC patient glycan node data in FIG. 16A-FIG. 16D. It is notable that glycan nodes from the smoking-matched (SM) cancer-free controls from this lung cancer study are quite similar in their overall distributions to the NED, NMIBC and MIBC patients in the present UCC study—and yet are strikingly elevated above the certifiably healthy controls.²¹ As previously described,²¹ smoking status (provided as “never-”, “former-” or “current smoker”) within this control group had a minor but statistically significant impact on outer-arm and total fucosylation as well as β1-6 branching, but not on α2-6 sialylation or β1-4 branching. Yet smoking alone did not completely account for the elevation of glycan nodes in this control cohort relative to the certifiably healthy controls. Correspondingly, even in remission, most former UCC patients with no evidence of disease (NED), tended to maintain modestly elevated blood plasma glycan levels—wherein the NED patients with the most highly elevated levels were most likely to experience relapse (FIG. 14A-FIG. 14B).

Though the reason(s) for glycan node elevation in nominally cancer-free individuals are not fully known, it has previously been shown that serum glycans can be elevated in inflammatory patient states in the absence of cancer.²²⁻²⁴ Moreover, chronic inflammation is known to be closely associated with the development of cancer.⁴²⁻⁴⁴ Together with the observations presented here, this suggests that the elevated plasma glycan levels observed in former UCC patients (currently in the NED state) that are prognostic of recurrence may be driven by or simply part of inflammatory processes. To assess this possibility, we measured CRP concentrations and found them to be strongly significantly correlated with levels of both α2-6 sialylation and β1-6 branching (FIG. 15A-FIG. 15B)—an observation that goes hand-in-hand with the fact that CRP has been found to predict UCC patient survival.^{26,28}

This brings up the question of whether there is a mechanistic connection between alterations in plasma glycans (associated with inflammation) and the development or progression of cancer. There is evidence for the concept that the biological landscape experiences “grooming” or premetastatic “niche” formation prior to cancer establishing residence within the body.⁴⁵⁻⁴⁹ And while glycans are not solely responsible for this process, evidence exists that they play important roles. As previously summarized²¹ and others have explained in detail, cell-surface glycans that facilitate resistance of galectin-mediated apoptosis^{48,50-52} (including poly-N-acetyllactosamine modified core 2 O-glycans⁵³⁻⁵⁸) as well as sialylated glycans that stimulate the inhibitory Siglec-7 receptor on natural killer cells⁵³⁻⁵⁶ have important roles to play in helping cancer evade the body's natural immunity.

The results of Example 2 show that, relative to healthy individuals, there is a significant alteration of P/S glycan features that correlates with inflammation and is present at the onset of UCC—but that, unlike other types of cancer that we have observed to date²¹, does not change in a stage-dependent manner—even when UCC patients go into remission. Certifiably healthy individuals cannot be considered to be clinically relevant controls for the development of cancer diagnostics—but they do illustrate the striking changes in blood biochemistry that occur as cancer develops and takes hold in the human body. Thus taken together with Example 1, the findings of Example 2 suggest that if there are clinical applications for P/S glycan node measurements, they most likely lie in evaluating cancer patient relapse or progression risk—or in monitoring nominally healthy persons who exhibit behaviors such as smoking that put them at risk for the biochemical transition between a genuinely healthy state and one in which their blood chemistry (above and beyond mere behavior) reveals a truly high-risk state.

### Conclusions

α2-6 sialylation, β1-4 branching, β1-6 branching, and outer-arm fucosylation were found to be significantly elevated in both current and former (in remission) UCC patients relative to certifiably healthy living kidney donors, with ROC curve c-statistics averaging approximately 0.8—yet this does not make them clinically relevant diagnostic biomarkers of UCC. Differences between patients with muscle invasive UCC, non-muscle invasive UCC and patients in remission were not statistically significant. For UCC patients in remission, α2-6 sialylation and β1-6 branching were prognostic indicators of recurrence and were correlated with CRP levels (r=0.34 & 0.38, resp.; p<0.001), a known prognostic marker in UCC. Results highlighted the pronounced difference between the serum glycan biochemistry of healthy individuals vs. any stage of UCC (including remission) and underscored the concept that for plasma glycans the transition between a healthy state and an at-risk state is much more pronounced than that between an at-risk state and early stage cancer.

### Example 3

Materials. Heavy, stable-isotope-labeled D-glucose (U-¹³C₆, 99%; 1,2,3,4,5,6,6-D7, 97-98%) was obtained from Cambridge Isotope Laboratories (Tewksbury, Mass.). Acetone was acquired from Avantor Performance Materials (Center Valley, Pa.). Methanol was purchased from Honeywell Burdick & Jackson (Muskegon, Mich.). Acetonitrile and methylene chloride were obtained from Fisher Scientific (Fair Lawn, N.J.). Dimethyl sulfoxide (DMSO), iodomethane (99%, Cat. No. 18507), chloroform, trifluoroacetic acid (TFA), ammonium hydroxide, sodium borohydride, acetic anhydride, sodium acetate, and sodium hydroxide beads (20-40 mesh, Cat. No. 367176) were acquired from Sigma-Aldrich. Pierce spin columns (900 μL volume) were purchased from ThermoFisher Scientific (Waltham, Mass., Cat. No. 69705). GC-MS autosampler vials and Teflon-lined pierceable caps were obtained from Thermo-Fisher Scientific. GC consumables were acquired from Agilent (Santa Clara, Calif.); MS consumables were obtained from Waters (Milford, Mass.).

Plasma and Serum Samples. All specimens were collected in compliance with the Declaration of Helsinki principles. Once collected, they were coded and deidentified to protect patient identities.

Women Epidemiology Lung Cancer (WELCA) Set. EDTA plasma samples from stage I-IV lung cancer patients and age-matched controls were collected at 12 different collection centers in France.²⁶ This study was approved by the Institutional Review Board of the French National Institute of Health and Medical Research and by the French Data Protection Authority (IRB-Inserm, no. 3888 and CNIL no. C₁₃₋₅₂). As part of the WELCA Study, all-female lung cancer patients were recruited between September 2014 and December 2017, and age-matched all-female controls were recruited between June 2015 and December 2017. All women living in Paris and the lle de France area, newly diagnosed with lung cancer, were considered as eligible cases. Age-matched controls were randomly sampled from women living in the same area without a history of lung cancer. All peripheral blood samples were drawn and processed following a written standardized protocol.²⁶ Briefly, after transport to the laboratory at 4° C., blood samples collected in tubes containing EDTA additive were spun for 15 min at 3000 rpm and 4° C. in a standard centrifuge. Then the collected plasma samples were aliquoted and periodically transported on dry ice to the central repository for final storage at −80° C. No freeze-thaw cycles occurred prior to shipment to Arizona State University (Borges lab) for analysis. A detailed profile of the clinical characteristics of the patients in this WELCA study is given in Table S1 of Hu et al., J. Proteome Res. 2019 Nov. 1; 18(11):3985-3998.

Dual Gender Lung Cancer Set. Sodium heparin plasma samples from a lung cancer study consisting of patients and controls in both genders were collected by Dr. Xifeng Wu at the University of Texas MD Anderson Cancer Center. Even though it is a glycosaminoglycan itself, heparin possesses monomer units that are predominately carboxylated, sulfated or both, and thus cannot be directly detected by the analytical methodology used in this study. As reported previously, there are only negligible differences between glycan nodes measure in heparin plasma vs EDTA plasma or serum,¹⁹ and thus direct comparisons were made for these three types of biospecimens. Venous blood samples were collected from newly diagnosed and histologically confirmed lung cancer patients prior to therapy at the MD Anderson Cancer Center hospital. Blood samples of age-, gender-, smoking-, and ethnicity-matched controls were collected at the Kelsey-Seybold Clinic. All blood samples were collected since 1995 and processed following the same SOP. These specimens has previously been described.¹⁹

Stage I-Only Lung Cancer Set (Also Dual Gender). Serum samples for dual gender stage I lung adenocarcinoma patients were collected together with age-, gender-, and smoking-statusmatched controls, under NYU IRB approval at the NYU Langone Medical Center by Dr. Harvey Pass. Arterial blood samples were drawn from fasting patients undergoing surgery between September 2006 to August 2013 to remove one or more lung nodules that were detected during a CT scan. A pathological exam of the excised nodules was performed to determine whether nodules were benign or malignant. Serum was collected under a standardized procedure. These specimens have previously been described.¹⁹

Plasma Samples for the Stability Study. The samples employed for the ex vivo thawed-state stability study included EDTA plasma samples from three healthy male and two healthy female donors. These samples were aliquoted and stored at different temperatures over the course of a year, with their matched control aliquots stored continuously at −80° C. The mistreatment conditions included 10 days at −20° C., 90 days at −20° C., 360 days at −20° C., 2 days at 4° C., 90 days at 4° C., and 1 day at 25° C. At the end of the 360-day time point, glycan node analysis was performed on all the mistreated sample aliquots and their matched control aliquots.

Additional Biospecimen Details. A summary of the case-control sample sets discussed in this study is provided in Table S2 of Hu et al., J. Proteome Res. 2019 Nov. 1; 18(11):3985-3998. A 300 mL plasma sample from an individual donor was obtained from BioIVT, which served as a quality control sample to ensure batch-to-batch quantitative reproducibility. All specimens were stored at −80° C. prior to analysis.

### Experimental Procedures

The glycan node analysis procedure was adapted from Borges et al.^{16,17}

Permethylation, Nonreductive Release, and Purification of Glycans. Nine microliters (9 μL) of blood plasma and 1 μL of a 5 mMsolution of heavy-labeled D-glucose (U-¹³C₆, 99%; 1,2,3,4,5,6,6-D7, 97-98%) and N-acetyl-D-[UL-13C6]-glucosamine were mixed in a 1.5 mL Eppendorf tube, followed by the addition of 270 μL of DMSO. About 0.7 g sodium hydroxide beads were collected in a Pierce spin column (900 μL volume) and washed once with 350 μL of acetonitrile (ACN) followed by two rinses with 350 μL of DMSO. The plasma sample was mixed in with 270 μL of DMSO and 105 μL of iodomethane followed by immediate mixing. The whole mixture was then added to the preconditioned NaOH beads in the plugged microfuge spin column. After occasional gentle stirring the sample solution in NaOH column for 11 min, the microfuge spin column was unplugged and spun for 30 s at 5000 rpm (1000 g in a fixed-angle rotor). The collected sample solution was quickly transferred into 3.5 mL of 0.5MNaCl solution in 0.2 M sodium phosphate buffer (pH 7) within a silanized 13×100 mm glass test tube. To maximize glycan recovery, the NaOH beads were then washed twice by 300 μL of ACN, with all spin-throughs immediately transferred into the same silanized glass test tube. To perform liquid/liquid (L/L) extraction, 1.2 mL of chloroform was added to each test tube, which was then capped and shaken well. After brief centrifugation to separate the layers, the aqueous layer (top) was discarded and then replaced by a fresh aliquot of 3.5 mL of 0.5 M NaCl solution in 0.2 M sodium phosphate buffer (pH 7). After three L/L extraction rounds, the chloroform layer was finally recovered and dried under a gentle stream of nitrogen in a heater block set to 74° C.

Hydrolysis, Reduction, and Acetylation. To perform TFA hydrolysis, each sample was mixed with 2MTFA (325 μL) and incubated at 121° C. for 2 h, which was then dried under a gentle stream of nitrogen in a heater block set to 74° C. To reduce the sugar aldehydes, each sample was incubated at room temperature for 1 h after dissolution in 475 μL of freshly made 10 mg/mL sodium borohydride in 1 M ammonium hydroxide. To remove excess borate, 63 μL of methanol (MeOH) was added and dried under nitrogen, followed by adding 125 μL of 9:1 (v/v) MeOH:acetic acid. Samples were then dried under nitrogen and then fully dried in a vacuum desiccator for 20 min. The last step is acetylation of nascent hydroxyl groups, in which 18 μL of deionized water was added to each test tube to dissolve any precipitates. After adding 250 μL of acetic anhydride and sonicating in a water bath for 2 min, each sample was incubated for 10 min at 60° C., followed by mixing with 230 μL of concentrated TFA and incubated again at 60° C. for 10 min. To clean up the sample mixture, L/L extraction was performed twice after adding 1.8 mL of dichloromethane and 2 mL of deionized water to each test tube. With the aqueous layer (top layer) discarded for each round, the organic layer of each sample was then transferred to a silanized autosampler vial, dried under nitrogen and reconstituted in 120 μL of acetone, which was then capped in preparation for injection onto the GC-MS.

Gas Chromatography-Mass Spectrometry. An Agilent Model A7890 gas chromatograph (equipped with a CTC PAL autosampler) coupled to a Waters GCT (time-of-flight) mass spectrometer was employed to analyze the prepared samples. For each sample, 1 μL of the 120 μL total volume was injected onto a hot (280° C.), silanized glass liner (Agilent Cat. No. 5183-4647) containing a small plug of silanized glass wool at a split ratio of 20:1. A 30-m DB-5 ms GC column was used to separate different sample components, facilitated by the carrier gas (helium) with a 0.8 mL/min flow rate. The GC oven temperature was initially kept at 165° C. for 0.5 min, then increased to 265° C. at a rate of 10° C./min, followed by immediate ramping to 325° C. at a rate of 30° C./min, and finally held at 325° C. for 3 min. Sample components eluted from GC column were subjected to electron ionization (70 eV, 250° C.). Positive-ion mode mass spectra from individual TOF pulses over a m/z range of 40-800 were summed every 0.1 s. Daily tuning and calibration of the mass spectrometer was performed with perfluorotributylamine to ensure reproducible relative abundances of EI ions and mass accuracy within 10 ppm.

### Data Analysis

Data Processing. Quanlynx 4.1 software was employed to integrate the summed extracted-ion chromatogram (XIC) peak areas for all glycan nodes. The peak areas were automatically integrated and manually verified, then exported to a spreadsheet for further analysis.

Two possible normalization approaches were considered: (1) individual hexoses were normalized to heavy glucose, and individual N-acetylhexosamines (HexNAcs) were normalized to heavy N-acetyl glucosamine (GlcNAc); (2) individual hexoses were normalized to the sum of all endogenous hexoses, and individual HexNAcs were normalized to the sum of all endogenous HexNAcs. The second normalization approach tends to provide better interbatch reproducibility (<9% average CV for the six most elevated glycan nodes), but the first approach performs better in identifying the potential increases of all glycan nodes in the patient groups relative to the control group while maintaining a reasonable interbatch % CV (i.e., <21%). Thus, results reported below are based on normalization with heavy glucose and heavy GlcNAc, unless otherwise stated. The raw data of all XIC peak areas for all samples, together with the normalized data by the two normalization approaches and % CV values for batch-to-batch quality control (QC) samples are provided in a spreadsheet available as Supporting Information of Hu et al., J. Proteome Res. 2019 Nov. 1; 18(11):3985-3998.

### Statistical Analysis

For the glycan node data of each cohort, outliers were removed by log-transformation and the ROUT method at Q=1% using GraphPad Prism 7. Outlier-removed data were then reverse transformed by taking the antilog of each value. To identify differences between cohorts, the Kruskal-Wallis test followed by the Benjamini-Hochberg false discovery correction procedure was performed at a 5% false discovery rate using GraphPad Prism 7. RStudio Version 1.0.143 was used to compare different receiver operating characteristic (ROC) curves by Delong's test or Bootstrap test. The ROC curves shown in figures were plotted by GraphPad Prism 7. Correlation of glycan nodes with age or smoking pack-years were assessed via Spearman's rank correlation in GraphPad Prism 7. Stage-bystage multivariate modeling was performed using multivariate logistic regression in RStudio Version 1.0.143, with assessment carried out by leave-one-out-validation, and model selection done using a best subsets procedure. The ability of specific glycan nodes to predict lung cancer survival was evaluated with Cox proportional hazards regression model in SAS 9.4. And GraphPad Prism 7 was applied to generate survival curves and perform associated log-rank Mantel-Cox tests.

### Results

Striking increases in glycan nodes that serve as direct indicators of α2-6 sialylation, β1-4 branching, β1-6 branching, and antennary fucosylation in stage III-IV lung cancer. Similar increases also observed for 2-linked mannose and 4-linked N-acetylglucosamine, both of which are associated with total glycosylation levels, especially N-glycans (FIG. 3 of Hu et al., J. Proteome Res. 2019 Nov. 1; 18(11):3985-3998).
Significant increases in these glycan nodes in stage I-II lung cancer, with a general trend for increasing prevalence from stage I-IV (FIG. 3 of Hu et al., J. Proteome Res. 2019 Nov. 1; 18(11):3985-3998).
Minimal dependence of glycan nodes on smoking status, age, and histological type of lung cancer.
The top quartiles of all six glycan nodes listed in bullet point 1 above predict all-cause mortality across all stages of lung cancer combined (FIG. 6 of Hu et al., J. Proteome Res. 2019 Nov. 1; 18(11):3985-3998).
Glycan nodes corresponding to α2-6 sialylation and β1-4 branching are particularly good at predicting survival in stage IV patients (Figure S6 of Hu et al., J. Proteome Res. 2019 Nov. 1; 18(11):3985-3998).

### Glycan Node Stability in Plasma

Cancer patient enrollment for the WELCA study took place at 12 different sites. In some cases, samples were permitted to sit overnight at 4° C. prior to final processing and storage at −80° C. In other cases, sample aliquots were temporarily stored at −20° C. prior to shipment a few weeks later to the central repository where they were kept long-term at −80° C. As such, assessment of the stability of glycan nodes in EDTA plasma kept at room temperature, 4° C., and −20° C. for varying lengths of time was assessed.

Five EDTA plasma samples from separate healthy donors (three male and two female), were aliquoted and temporarily kept at −20° C. for 10, 90, and 360 days, 4° C. for 2 or 90 days, room temperature for 1 day, or kept continuously at −80° C. Samples kept temporarily at temperatures warmer than −80° C. were compared with their respective control aliquots kept continuously at −80° C. The glycan nodes that are typically present at >1% relative abundance within their respective hexose or HexNAc class were measured and normalized to heavy, stable isotope-labeled glucose and GlcNAc internal standards or, alternatively, normalized to the sum of endogenous hexoses or HexNAcs. No significant differences were observed in the data sets normalized to the sum of endogenous hexoses/HexNAcs. When normalized to heavy, stable isotope-labeled glucose and GlcNAc internal standards, the only significant difference observed was an increase in 6-linked galactose for samples stored at room temperature for 1 day (p=0.033; Table S3 of Hu et al., J. Proteome Res. 2019 Nov. 1; 18(11):3985-3998). Thus, under the mildly adverse conditions to which some of the specimens in this study may have been exposed (less than a day at 4° C. or up to a few weeks at −20° C.), glycan nodes were found to be stable.

Notably, a study of the impact of plasma vs serum matrices on glycan nodes was previously reported in this journal.¹⁹ Differences observed were modest and did not impact the biological results of either the previous study or this one.

### Altered Glycan Features in Stage I-IV Patients

Basic clinical characteristics and n-values of the WELCA sample set were described in the Materials and Methods section and Table S1 of Hu et al., J. Proteome Res. 2019 Nov. 1; 18(11):3985-3998. All 207 control and 208 stage I-IV patient samples were randomized and analyzed in 27 batches. Within each control and case sample, a total of 19 glycan “nodes” were measured. The relative abundances of each of these nodes contributed at least 1% of the total hexose or total Nacetylhexosamine (HexNAc) signal. Data from each of the 19 glycan nodes were normalized to heavy, isotope-labeled glucose and GlcNAc internal standards. Statistically significant differences were detected in each cancer stage relative to the control cohort: 10, 6, 18, and 19 out of 19 glycan nodes were increased in stage I, II, III, and IV, respectively (Table 1 of Hu et al., J. Proteome Res. 2019 Nov. 1; 18(11):3985-3998). Data for each glycan node normalized to the sum of endogenous hexoses or HexNAcs were analyzed analogously (Table S4 of Hu et al., J. Proteome Res. 2019 Nov. 1; 18(11):3985-3998). This revealed shifts in glycan compositions in stage I-IV patients vs controls. However, because quantitative changes in glycans tended to outpace glycan compositional changes (as previously observed¹⁹) this normalization procedure was not as sensitive in distinguishing age-matched controls from lung cancer patients at each stage.

Six glycan nodes were found to be significantly elevated at nearly every stage in lung cancer patients relative to the age-matched controls, and these included: 2-linked mannose (2-Man) and 4-linked N-acetylglucosamine (4-GlcNAc), both of which are associated with total glycosylation levels especially for N-glycans;¹⁴ 6-linked galactose, corresponding to α2-6 sialylation;¹⁶ 2,4-linked mannose, corresponding to β1-4 branching;¹⁶ 2,6-linked mannose, corresponding to β1-6 branching;¹⁶ and 3,4-linked GlcNAc, which primarily corresponds to antennary fucosylation¹⁶ (FIG. 3 of Hu et al., J. Proteome Res. 2019 Nov. 1; 18(11):3985-3998). The latter four nodes were among the top five most elevated nodes in a previously reported lung cancer study.¹⁹ The receiver operating characteristic (ROC) curve c-statistics (areas under the curve, AUCs) for these six glycan nodes in stage I-IV patients vs controls ranged (with two exceptions) from 0.68 to 0.92 (FIG. 3 of Hu et al., J. Proteome Res. 2019 Nov. 1; 18(11):3985-3998).

For most of these six glycan nodes there were significant differences between stages (FIG. 3 of Hu et al., J. Proteome Res. 2019 Nov. 1; 18(11):3985-3998), but the most robust differences tended to be between stage IV and stage I-II patients. 2,4-Man, the glycan node indicative of β1-4 branching, was the best at differentiating stage IV vs all other stages of lung cancer. ROC curves showing the ability of β1-4 branching to distinguish between stage IV and all other stages are provided in Figure S1 of Hu et al., J. Proteome Res. 2019 Nov. 1; 18(11):3985-3998.

### Prominent Early-Stage Alteration

Relative to the age-matched controls, five of the six top performing glycan node markers in stage I patients, and four in stage II patients, were significantly increased (FIG. 3a-f of Hu et al., J. Proteome Res. 2019 Nov. 1; 18(11):3985-3998). In addition, theROCc-statistics (AUC) of these glycan nodes were mostly statistically significant and ranged from 0.68 to 0.80 (with one exception). The notable alterations of glycan nodes in early stages were not previously observed for other lung cancer sets, such as the dual gender lung cancer set and stage I-only lung cancer set (which was also dual gender) reported in previous work¹⁹ (n-values for these studies are provided in Table S2 of Hu et al., J. Proteome Res. 2019 Nov. 1; 18(11):3985-3998). Stage-specific ROC curves from the WELCA study and these other two studies were statistically compared (Table S5 of Hu et al., J. Proteome Res. 2019 Nov. 1; 18(11):3985-3998) and are shown side-by-side in FIG. 4 of Hu et al., J. Proteome Res. 2019 Nov. 1; 18(11):3985-3998. Significant differences were observed for β1-6 branching when the ROC curve of the stage I cohort of the WELCA set was compared to that of the stage I only lung cancer set and that of the stage I cohort of the dual gender lung cancer set. When comparing ROC curves for the stage IV cohorts of the WELCA set and the dual gender lung cancer set, significant differences were found for three glycan features including α2-6 sialylation, β1-4 and β1-6 branching. Since all the lung cancer patients and age-matched controls involved in the WELCA set were female, the gender dependence of these glycan node markers in early stages was evaluated in the other two lung cancer sets, which included patients and controls from both sexes. When sample set and stage were held constant, the ROC curves of the two sexes were compared for each glycan node using Delong's test or the Bootstrap test (Table S6 of Hu et al., J. Proteome Res. 2019 Nov. 1; 18(11):3985-3998). No significant differences were observed, however, indicating the early stage clinical performance characteristics of the six glycan node markers were independent of gender.

### Negligible Dependence on Smoking-Status, Age, and Histological Type

No significant alteration of five out of the six top performing glycan node markers was observed when each individual glycan node was separately analyzed for differences among never-smokers, previous smokers and current smokers within the WELCA study control cohort. The only exception was 3,4-linked GlcNAc (corresponding to antennary fucosylation), which was slightly elevated in current smokers relative to previous smokers (Figure S2 of Hu et al., J. Proteome Res. 2019 Nov. 1; 18(11):3985-3998). Spearman's rank correlation analysis demonstrated no statistically significant correlation with smoking pack-years in the control cohort, both for all control patients and control patients with smoking history (smoking pack-year >0). Together, these data revealed that the top performing glycan node markers within the control cohort had negligible dependence on smoking status. (A parallel analysis within the cancer patient cohort was not conducted due to the confounding correlation between smoking and lung cancer.)

The average ages of the control and case cohorts were nearly identical (61.2 and 61.6, respectively; Table S1). After pooling all data from the cases (all stages) and controls, 3,4-linked GlcNAc, corresponding to antennary fucosylation, was found to be weakly correlated with age (correlation coefficient r=0.159, p=0.0016; FIG. 5a of Hu et al., J. Proteome Res. 2019 Nov. 1; 18(11):3985-3998). When the control and case cohorts were analyzed separately, a significant correlation with age for 3,4-linked GlcNAc was only observed in the control cohort (correlation coefficient r=0.205, p=0.0031). No statistically significant correlations with age were observed for the other five top performing glycan nodes (Table S7 of Hu et al., J. Proteome Res. 2019 Nov. 1; 18(11):3985-3998). When the population was divided into smaller age groups, only 3,4-linked GlcNAc showed significant differences between pairs of decades; if the control and case cohorts were investigated in isolation, 3,4-linked GlcNAc within the controls in particular indicated a distinct upward pattern in more advanced age groups (FIG. 5b of Hu et al., J. Proteome Res. 2019 Nov. 1; 18(11):3985-3998). The same phenomenon was observed in the male-only controls of the dual gender lung cancer set. However, with the exception of 3,4-GlcNAc, these analyses indicated a lack of dependence of glycan node markers on age.

The effect of lung cancer histological subtypes on the six glycan nodes was evaluated in the stage IV non-small-cell lung cancer (NSCLC) subcohort (i.e., the largest single-stage subcohort available; Table S1 of Hu et al., J. Proteome Res. 2019 Nov. 1; 18(11):3985-3998). For each glycan node marker, ROC curves of the three histological subtypes of NSCLC-adenocarcinoma, squamous cell carcinoma, and large cell Carcinoma-were compared pairwise by Delong's test or Bootstrap test (Figure S3, Table S8 of Hu et al., J. Proteome Res. 2019 Nov. 1; 18(11):3985-3998). No statistically significant differences between histological subtypes of NSCLC were discovered for any glycan node marker.

These findings on glycan node independence from smoking status, age and histological type are consistent with previously reported findings from other lung cancer case/control studies.¹⁹

### Role of Gender in Defining Plasma Glycans

The WELCA studied consisted entirely of women. Thus, to evaluate the role of gender in plasma glycan nodes, we turned to control patient data from the “large lung cancer” cohort of a previous study.¹⁹ This set of cancer-free patients consisted of plasma samples from 123 males and 76 females. Since it was not previously done, we looked for gender differences in all 19 glycan nodes evaluated in the WELCA study and found significant decreases in 3,4-linked GlcNAc (the node that corresponds to antennary fucosylation) as well as total fucose in females relative to males-regardless of whether data were normalized to heavy Glc/GlcNAc or to the sum of endogenous hexoses/HexNAcs (p<0.05 or lower after applying the Benjamini-Hochberg false discovery correction procedure). These observed increases in antennary fucosylation agree with previously published findings on studies of women of approximately the same age.^{28,29} Moreover, in the WELCA study we found that the observed increase in antennary fucosylation with age (FIG. 5 of Hu et al., J. Proteome Res. 2019 Nov. 1; 18(11):3985-3998) agreed with that previously observed by Reiding et al.²⁹

Notably, we also observed increases in 2,4-linked mannose, corresponding to β1-4 branching, in women compared to men (p<0.05 for both heavy Glc/GlcNAc and endogenous normalizations). These findings align with those from Knežević et al.²⁸ and Reiding et al.²⁹ in which they found modest increases in triantennary and tetrantennary glycans in women relative to men-though for this glycan feature only the study of Knežević et al. revealed a statistically significant difference.²⁸

### Total Glycosylation and Multivariate Model of Glycan Features

The clinical performance characteristics of total glycosylation (i.e., total hexoses, total HexNAcs, and the sum of total hexoses and total HexNAcs) were evaluated and compared to individual glycan node markers on a stage-by-stage basis (Table S9 of Hu et al., J. Proteome Res. 2019 Nov. 1; 18(11):3985-3998). Results of ROC curve comparisons by paired Delong's tests demonstrated that total glycosylation cannot distinguish stage I-IV cases from controls better than individual glycan node markers.

Additionally, multivariate logistic regression models were built and compared with the clinical performance characteristics of individual glycan nodes at each stage (Figure S4 of Hu et al., J. Proteome Res. 2019 Nov. 1; 18(11):3985-3998). Fully cross-validated multivariate logistic regression models were no better at detecting lung cancer than the top-performing individual glycan node at each respective stage. Again, these results were consistent with previous observations in lung cancer.¹⁹

### Prediction of All-Cause Mortality

To evaluate the ability of the six glycan nodes to predict all-cause mortality, glycan node data were broken into quartiles and analyzed by Cox proportional hazards regression, with adjustment for age, smoking status, and cancer stage (Table S10 of Hu et al., J. Proteome Res. 2019 Nov. 1; 18(11):3985-3998). First and foremost, for patients in all four stages, the top quartiles of all six glycan node markers predicted all-cause mortality with hazard ratios in the range of 2-3 and p<0.01, relative to all other quartiles combined. The different rates of death for the top quartile versus all other quartiles for each glycan node marker are illustrated by survival curves (FIG. 6 of Hu et al., J. Proteome Res. 2019 Nov. 1; 18(11):3985-3998).

When focusing on stage III and IV patients, the top quartiles of all six glycan node markers predicted all-cause mortality with hazard ratios in the range of 2-3 and p<0.05 (Table S10 of Hu et al., J. Proteome Res. 2019 Nov. 1; 18(11):3985-3998) relative to all other quartiles combined (survival curves shown in Figure S5 of Hu et al., J. Proteome Res. 2019 Nov. 1; 18(11):3985-3998). Similar results were observed for stage IV patients only (Table S10 of Hu et al., J. Proteome Res. 2019 Nov. 1; 18(11):3985-3998). However, when stage III patients were analyzed alone, the hazard ratios of all six glycan nodes were not significantly different from 1 (p>0.05), indicating the relative risk of death was not detectably different between patients in the top quartile vs all other quartiles of each glycan node. 6-linked galactose (corresponding to α2-6 sialylation) and 2,4-linked mannose (corresponding to β1-4 branching) were significantly different between stages III and IV (FIG. 3b,c). Stage-specific survival curves for these glycan nodes are provided in Figure S6 of Hu et al., J. Proteome Res. 2019 Nov. 1; 18(11):3985-3998.

Overall, these results for glycan node-based prediction of mortality vary slightly, but are largely consistent with previously reported results on the ability of α2-6 sialylation and branched mannose residues to predict all-cause mortality in lung cancer.

### Discussion

### Consistency of Specific Glycan Feature Changes in Lung Cancer

Six out of 19 quantified glycan nodes, corresponding to total glycosylation levels (especially for N-glycans), α2-6 sialylation, β1-4 branching, β1-6 branching, and antennary fucosylation, were significantly elevated in the WELCA lung cancer patients relative to age-matched controls. These findings in the WELCA set are highly consistent with our previously reported lung cancer study on a dual gender lung cancer set,⁹ which also demonstrated the distinct increase of the latter four glycan features within stage III-IV cases compared to their respective control cohorts.

Our observations of the glycan node-based feature changes in lung cancer patients are closely aligned with the intact glycan changes reported in lung cancer by Vasseur and colleagues.³⁰ Their intact glycan analysis results primarily revealed significant increases in antennary fucosylation, as well as fucosylated tri- and tetra-antennary N-glycans-findings that are in line with increases observed here in β1-4 branching and β1-6 branching.

### Consistency in Prediction of Survival

The six top performing glycan nodes-based features in this study were not only able to distinguish lung cancer patients from age-matched controls, but were also able to predict all-cause mortality in the WELCA set-a finding that agrees well with the survival-predicting nodes in our previously reported study on the dual gender lung cancer set.¹⁹ Similar discoveries regarding the prognostic capacity of P/S glycans have also been reported by other groups. Hashimoto and colleagues³¹ suggested that specific glycoforms of serum al-acid glycoprotein (AGP) seemed to predict progression and mortality of several carcinomas, including lung cancer. According to their follow-up studies, patients who had the AGP glycoforms that contained highly fucosylated and branched sugar chains tended to have a poor prognosis. Besides the glycan features discussed above, another good prognostic predictor of lung cancer is the sialyl Lewis X epitope (SLex),³² which consists of α2-3 sialylation instead of α2-6 sialylation. The progression and survival in nonsmall-cell³³⁻³⁵ and small-cell lung cancer³⁶ can both be predicted by SLex.

Most clinical trials require that enrolled patient life expectancy exceed three months such that a benefit from treatment can be observed-yet formal guidelines are generally not provided to facilitate this prediction.³⁷ Glycan nodes representing α2-6 sialylation and β1-4 branching both performed well as prognostic indicators of survival within stage IV patients (Figure S6 of Hu et al., J. Proteome Res. 2019 Nov. 1; 18(11):3985-3998), and as such they may be able to provide some clinical utility toward this end.

### Early Stage Changes in Glycan Nodes

Unlike the other two lung cancer sets that reported on previously,¹⁹ some glycan node-based features were substantially altered in the WELCA lung cancer patients at stages I-II (FIG. 4 of Hu et al., J. Proteome Res. 2019 Nov. 1; 18(11):3985-3998). Even though a relatively low number of early stage samples were measured (n=16 and 13 for stage I and II, respectively), statistically significant elevations were detected in most of the six glycan node markers, alongside comparatively high ROC c-statistics. Outside of a statistical anomaly, there are two possible noncancer related causes for this phenomenon. First, since the lung cancer patients and controls enrolled in the WELCA study are all female, a distinct gender dependence of glycan features may exist, especially in early stages. However, this possibility was not evidenced by the observation that no significant difference was detected between men and women in stage I and II of the dual gender lung cancer set, as well as in the stage I-only lung cancer set, which was also dual gender (Table S6 of Hu et al., J. Proteome Res. 2019 Nov. 1; 18(11):3985-3998). The second possible explanation is that the nonsmoking-matched controls of the WELCA set may have lower relative abundances of all the glycan nodes of interest relative to the smoking-matched controls for other lung cancer sets. In the WELCA set most controls were never-smokers, but the cancer patients were mainly current-smokers (Table S1 of Hu et al., J. Proteome Res. 2019 Nov. 1; 18(11):3985-3998), suggesting that smoking history might possibly contribute to increases in some glycan nodes. Taken together with the observation that the top performing glycan node markers within the control cohort had near-negligible dependence on smoking status (Figure S2 of Hu et al., J. Proteome Res. 2019 Nov. 1; 18(11):3985-3998), smoking appears to contribute to slight, but mostly statistically insignificant elevation of glycan nodes. Smoking is undoubtedly bad for the liver,³⁸ which secretes approximately half of all circulating glycoproteins.^{39,40} Nevertheless, these results that indicate only a mild contribution of smoking to alterations in circulating glycan nodes is in full agreement with results from a previous study of glycan nodes in lung cancer patients in which controls were smoking status-matched to the lung cancer patients, and in which only minor impacts of smoking on glycan nodes within the control population were observed.¹⁹

### Role of Gender

Many studies have reported important gender differences in lung cancer between men and women, in terms of histological type, tobacco exposure, and survival and treatment response.^{41,42} Here, by comparison with previously conducted studies,¹⁹ no obvious gender differences were detected with regard to P/S glycan features. Smoking is the primary risk factor for lung cancer. However, a large percentage of women with lung Adenocarcinoma-between 20% and 30% in Western countries and nearly 80% in Asian countries-are nonsmokers.²⁶ Hence, some female-specific risk factors for lung cancer must exist and may play vital roles in lung cancer development, progression and survival; these may include hormonal factors and occupational risk factors in female occupations—as suggested by Stticker et al.²⁶

### Conclusions

As represented by glycan nodes, blood plasma glycans were found to be stable under a variety of less-than-ideal sample storage conditions. The diagnostic and prognostic capacity of plasma glycan features in stage I-IV lung cancer-as represented by monosaccharide and linkage-specific glycan nodes-were validated in the WELCA case-control study. Significant elevation of α2-6 sialylation, β1-4 branching, β1-6 branching, antennary fucosylation, and total N-glycosylation level was observed in almost every stage of lung cancer relative to age-matched control groups. Early stage detection was stronger than previously observed,¹⁹ but this observation may have been related to the lack of smoking status-matching between cases and controls in the WELCA study. Nevertheless, alteration of glycan features in lung cancer was found to be almost completely independent of smoking status, age, and histological subtypes of lung cancer. The six most-elevated glycan features predicted all-cause mortality in lung cancer patients after adjusting for age, smoking status, and cancer stage. No gender-based differences were discovered in glycan features associated with lung cancer.

### REFERENCES

The following references are hereby incorporated by reference in their entireties:

### References Cited in the Cancer Section of Background and in Example 1:

1. Varki, A.; Kannagi, R.; Toole, B., Glycosylation Changes in Cancer. In Essentials of Glycobiology, 2nd ed.; Varki, A.; Cummings, R. D.; Esko, J. D.; Freeze, H. H.; Stanley, P.; Bertozzi, C. R.; Hart, G. W.; Etzler, M. E., Eds. Cold Spring Harbor Laboratory Press: Cold Spring Harbor, N.Y., 2009; pp 617-632.
2. Kailemia, M. J.; Park, D.; Lebrilla, C. B., Glycans and glycoproteins as specific biomarkers for cancer. Anal Bioanal Chem 2017, 409, (2), 395-410.
3. Lauc, G.; Pezer, M.; Rudan, I.; Campbell, H., Mechanisms of disease: The human N-glycome. Biochim Biophys Acta 2016, 1860, (8), 1574-82.
4. Almeida, A.; Kolarich, D., The promise of protein glycosylation for personalised medicine. Biochim Biophys Acta 2016, 1860, (8), 1583-95.
5. An, H. J.; Miyamoto, S.; Lancaster, K. S.; Kirmiz, C.; Li, B.; Lam, K. S.; Leiserowitz, G. S.; Lebrilla, C. B., Profiling of glycans in serum for the discovery of potential biomarkers for ovarian cancer. J Proteome Res 2006, 5, (7), 1626-35.
6. Ruhaak, L. R.; Miyamoto, S.; Lebrilla, C. B., Developments in the identification of glycan biomarkers for the detection of cancer. Mol Cell Proteomics 2013, 12, (4), 846-55.
7. Hajba, L.; Csanky, E.; Guttman, A., Liquid phase separation methods for N-glycosylation analysis of glycoproteins of biomedical and biopharmaceutical interest. A critical review. Analytica Chimica Acta 2016, 943, 8-16.
8. Frost, D. C.; Li, L., Recent advances in mass spectrometry-based glycoproteomics. Adv Protein Chem Struct Biol 2014, 95, 71-123.
9. Mechref, Y.; Hu, Y.; Garcia, A.; Hussein, A., Identifying cancer biomarkers by mass spectrometry-based glycomics. Electrophoresis 2012, 33, (12), 1755-67.
10. Hennig, R.; Cajic, S.; Borowiak, M.; Hoffmann, M.; Kottler, R.; Reichl, U.; Rapp, E., Towards personalized diagnostics via longitudinal study of the human plasma N-glycome. Biochim Biophys Acta 2016, 1860, (8), 1728-38.
11. Verhelst, X.; Vanderschaeghe, D.; Castera, L.; Raes, T.; Geerts, A.; Francoz, C.; Colman, R.; Durand, F.; Callewaert, N.; Van Vlierberghe, H., A Glycomics-Based Test Predicts the Development of Hepatocellular Carcinoma in Cirrhosis. Clinical Cancer Research 2017, 23, (11), 2750-2758.
12. Saldova, R.; Shehni, A. A.; Haakensen, V. D.; Steinfeld, I.; Hilliard, M.; Kifer, I.; Helland, A.; Yakhini, Z.; Borresen-Dale, A. L.; Rudd, P. M., Association of N-Glycosylation with Breast Carcinoma and Systemic Features Using High-Resolution Quantitative UPLC. J Proteome Res 2014, 13, (5), 2314-2327.
13. Hua, S.; An, H. J.; Ozcan, S.; Ro, G. S.; Soares, S.; DeVere-White, R.; Lebrilla, C.

B., Comprehensive native glycan profiling with isomer separation and quantitation for the discovery of cancer biomarkers. The Analyst 2011, 136, (18), 3663-71.
14. Hua, S.; Jeong, H. N.; Dimapasoc, L. M.; Kang, I.; Han, C.; Choi, J. S.; Lebrilla, C. B.; An, H. J., Isomer-specific LC/MS and LC/MS/MS profiling of the mouse serum N-glycome revealing a number of novel sialylated N-glycans. Anal Chem 2013, 85, (9), 4636-43.
15. Klasic, M.; Kristic, J.; Korac, P.; Horvat, T.; Markulin, D.; Vojta, A.; Reiding, K. R.; Wuhrer, M.; Lauc, G.; Zoldos, V., DNA hypomethylation upregulates expression of the MGAT3 gene in HepG2 cells and leads to changes in N-glycosylation of secreted glycoproteins. Sci Rep 2016, 6, 24363.
16. Kolarich, D.; Lepenies, B.; Seeberger, P. H., Glycomics, glycoproteomics and the immune system. Curr Opin Chem Biol 2012, 16, (1-2), 214-20.
17. Sethi, M. K.; Fanayan, S., Mass Spectrometry-Based N-Glycomics of Colorectal Cancer. Int J Mol Sci 2015, 16, (12), 29278-304.
18. Borges, C. R.; Rehder, D. S.; Boffetta, P., Multiplexed surrogate analysis of glycotransferase activity in whole biospecimens. Anal Chem 2013, 85, (5), 2927-36.
19. Zaare, S.; Aguilar, J. S.; Hu, Y.; Ferdosi, S.; Borges, C. R., Glycan Node Analysis: A Bottom-up Approach to Glycomics. J Vis Exp 2016, 111, e53961.
20. Hu, Y.; Borges, C. R., A spin column-free approach to sodium hydroxide-based glycan permethylation. The Analyst 2017, 142, (15), 2748-2759.
21. Ho, T. H.; Nateras, R. N.; Yan, H.; Park, J. G.; Jensen, S.; Borges, C.; Lee, J. H.; Champion, M. D.; Tibes, R.; Bryce, A. H.; Carballido, E. M.; Todd, M. A.; Joseph, R. W.; Wong, W. W.; Parker, A. S.; Stanton, M. L.; Castle, E. P., A Multidisciplinary Biospecimen Bank of Renal Cell Carcinomas Compatible with Discovery Platforms at Mayo Clinic, Scottsdale, Ariz. PLoS One 2015, 10, (7), e0132831.
22. Grizzle, W. E.; Gunter, E. W.; Sexton, K. C.; Bell, W. C., Quality management of biorepositories. Biopreservation and Biobanking 2015, 13, (3), 183-94.
23. Katchman, B. A.; Chowell, D.; Wallstrom, G.; Vitonis, A. F.; LaBaer, J.; Cramer, D. W.; Anderson, K. S., Autoantibody biomarkers for the detection of serous ovarian cancer. Gynecol Oncol 2017, 146, (1), 129-136.
24. Anderson, K. S.; Cramer, D. W.; Sibani, S.; Wallstrom, G.; Wong, J.; Park, J.; Qiu, J.; Vitonis, A.; LaBaer, J., Autoantibody signature for the serologic detection of ovarian cancer. J Proteome Res 2015, 14, (1), 578-86.
25. Katchman, B. A.; Barderas, R.; Alam, R.; Chowell, D.; Field, M. S.; Esserman, L. J.; Wallstrom, G.; LaBaer, J.; Cramer, D. W.; Hollingsworth, M. A.; Anderson, K. S., Proteomic mapping of p53 immunogenicity in pancreatic, ovarian, and breast cancers. Proteomics Clin Appl 2016, 10, (7), 720-31.
26. Murphy, B. M.; Swarts, S.; Mueller, B. M.; van der Geer, P.; Manning, M. C.; Fitchmun, M. I., Protein instability following transport or storage on dry ice. Nat Methods 2013, 10, (4), 278-9.
27. Borges, C. R.; Rehder, D. S.; Jensen, S.; Schaab, M. R.; Sherma, N. D.; Yassine, H.; Nikolova, B.; Breburda, C., Elevated Plasma Albumin and Apolipoprotein A-I Oxidation under Suboptimal Specimen Storage Conditions. Mol Cell Proteomics 2014, 13, (7), 1890-9.
28. Moloney, D. J.; Shair, L. H.; Lu, F. M.; Xia, J.; Locke, R.; Matta, K. L.; Haltiwanger, R. S., Mammalian Notch1 is modified with two unusual forms of O-linked glycosylation found on epidermal growth factor-like modules. Journal of Biological Chemistry 2000, 275, (13), 9604-9611.
29. Anderson, N. L.; Anderson, N. G., The human plasma proteome: history, character, and diagnostic prospects. Mol Cell Proteomics 2002, 1, (11), 845-67.
30. Baker, E. S.; Liu, T.; Petyuk, V. A.; Burnum-Johnson, K. E.; Ibrahim, Y. M.; Anderson, G. A.; Smith, R. D., Mass spectrometry for translational proteomics: progress and clinical implications. Genome Med 2012, 4, (8), 63.
31. Vasseur, J. A.; Goetz, J. A.; Alley, W. R., Jr.; Novotny, M. V., Smoking and lung cancer-induced changes in N-glycosylation of blood serum proteins. Glycobiology 2012, 22, (12), 1684-708.
32. Miura, Y.; Endo, T., Glycomics and glycoproteomics focused on aging and age-related diseases—Glycans as a potential biomarker for physiological alterations. Biochimica Et Biophysica Acta-General Subjects 2016, 1860, (8), 1608-1614.
33. Miyahara, K.; Nouso, K.; Saito, S.; Hiraoka, S.; Harada, K.; Takahashi, S.; Morimoto, Y.; Kobayashi, S.; Ikeda, F.; Miyake, Y.; Shiraha, H.; Takaki, A.; Okada, H.; Amano, M.; Hirose, K.; Nishimura, S.; Yamamoto, K., Serum glycan markers for evaluation of disease activity and prediction of clinical course in patients with ulcerative colitis. PLoS One 2013, 8, (10), e74861.
34. Vanderschaeghe, D.; Laroy, W.; Sablon, E.; Halfon, P.; Van Hecke, A.; Delanghe, J.; Callewaert, N., GlycoFibroTest Is a Highly Performant Liver Fibrosis Biomarker Derived from DNA Sequencer-based Serum Protein Glycomics. Molecular & Cellular Proteomics 2009, 8, (5), 986-994.
35. Callewaert, N.; Van Vlierberghe, H.; Van Hecke, A.; Laroy, W.; Delanghe, J.; Contreras, R., Noninvasive diagnosis of liver cirrhosis using DNA sequencer-based total serum protein glycomics. Nature Medicine 2004, 10, (4), 429-434.
36. Wysoczynski, M.; Ratajczak, M. Z., Lung cancer secreted microvesicles: underappreciated modulators of microenvironment in expanding tumors. Int J Cancer 2009, 125, (7), 1595-603.
37. Martins, V. R.; Dias, M. S.; Hainaut, P., Tumor-cell-derived microvesicles as carriers of molecular information in cancer. Current Opinion in Oncology 2013, 25, (1), 66-75.
38. Fontana, S.; Saieva, L.; Taverna, S.; Alessandro, R., Contribution of proteomics to understanding the role of tumor-derived exosomes in cancer progression: state of the art and new perspectives. Proteomics 2013, 13, (10-11), 1581-94.
39. Rabinovich, G. A.; Conejo-Garcia, J. R., Shaping the Immune Landscape in Cancer by Galectin-Driven Regulatory Pathways. Journal of Molecular Biology 2016, 428, (16), 3266-81.
40. Sethi, M. K.; Hancock, W. S.; Fanayan, S., Identifying N-Glycan Biomarkers in Colorectal Cancer by Mass Spectrometry. Acc Chem Res 2016, 49, (10), 2099-2106.
41. Hanahan, D.; Weinberg, R. A., Hallmarks of cancer: the next generation. Cell 2011, 144, (5), 646-74.
42. Wang, D.; DuBois, R. N., Immunosuppression associated with chronic inflammation in the tumor microenvironment. Carcinogenesis 2015, 36, (10), 1085-93.
43. Bleyer, A. J.; Reeves-Daniel, A. M. In A Population-Based Study of the U.S. Population Shows the Majority of Persons Cannot Donate due to Preventable Diseases and Socio-Economic Conditions, Kidney Week (American Society of Nephrology), Philadelphia, Pa., 2014; Journal of the American Society of Nephrology: Philadelphia, Pa., 2014; p 67A.
44. Arnold, J. N.; Saldova, R.; Galligan, M. C.; Murphy, T. B.; Mimura-Kimura, Y.; Telford, J. E.; Godwin, A. K.; Rudd, P. M., Novel glycan biomarkers for the detection of lung cancer. J Proteome Res 2011, 10, (4), 1755-64.
45. Ruhaak, L. R.; Stroble, C.; Dai, J. L.; Barnett, M.; Taguchi, A.; Goodman, G. E.; Miyamoto, S.; Gandara, D.; Feng, Z. D.; Lebrilla, C. B.; Hanash, S., Serum Glycans as Risk Markers for Non-Small Cell Lung Cancer. Cancer prevention research 2016, 9, (4), 317-323.
46. Alley, W. R., Jr.; Novotny, M. V., Glycomic analysis of sialic acid linkages in glycans derived from blood serum glycoproteins. J Proteome Res 2010, 9, (6), 3062-72.
47. Reiding, K. R.; Blank, D.; Kuijper, D. M.; Deelder, A. M.; Wuhrer, M., High-throughput profiling of protein N-glycosylation by MALDI-TOF-MS employing linkage-specific sialic acid esterification. Anal Chem 2014, 86, (12), 5784-93.
48. Holst, S.; Heijs, B.; de Haan, N.; van Zeijl, R. J.; Briaire-de Bruijn, I. H.; van Pelt, G. W.; Mehta, A. S.; Angel, P. M.; Mesker, W. E.; Tollenaar, R. A.; Drake, R. R.; Bovee, J. V.; McDonnell, L. A.; Wuhrer, M., Linkage-Specific in Situ Sialic Acid Derivatization for N-Glycan Mass Spectrometry Imaging of Formalin-Fixed Paraffin-Embedded Tissues. Anal Chem 2016, 88, (11), 5904-13.
49. Gryska, K.; Slupianek, A.; Laciak, M.; Gorny, A.; Mackiewicz, K.; Baumann, H.; Mackiewicz, A., Inflammatory cytokines controlling branching of N-heteroglycans of acute phase protein. Adv Exp Med Biol 1995, 376, 239-45.
50. Narisada, M.; Kawamoto, S.; Kuwamoto, K.; Moriwaki, K.; Nakagawa, T.; Matsumoto, H.; Asahi, M.; Koyama, N.; Miyoshi, E., Identification of an inducible factor secreted by pancreatic cancer cell lines that stimulates the production of fucosylated haptoglobin in hepatoma cells. Biochemical and Biophysical Research Communications 2008, 377, (3), 792-796.
51. Arnold, J. N.; Saldova, R.; Hamid, U. M.; Rudd, P. M., Evaluation of the serum N-linked glycome for the diagnosis of cancer and chronic inflammation. Proteomics 2008, 8, (16), 3284-93.
52. Saldova, R.; Wormald, M. R.; Dwek, R. A.; Rudd, P. M., Glycosylation changes on serum glycoproteins in ovarian cancer may contribute to disease pathogenesis. Disease Markers 2008, 25, (4-5), 219-232.
53. Sarrats, A.; Saldova, R.; Pla, E.; Fort, E.; Harvey, D. J.; Struwe, W. B.; de Llorens, R.; Rudd, P. M.; Peracaula, R., Glycosylation of liver acute-phase proteins in pancreatic cancer and chronic pancreatitis. Proteomics Clin Appl 2010, 4, (4), 432-48.
54. Cagnoni, A. J.; Perez Saez, J. M.; Rabinovich, G. A.; Marino, K. V., Turning-Off Signaling by Siglecs, Selectins, and Galectins: Chemical Inhibition of Glycan-Dependent Interactions in Cancer. Frontiers in oncology 2016, 6, 109.
55. Mendez-Huergo, S. P.; Blidner, A. G.; Rabinovich, G. A., Galectins: emerging regulatory checkpoints linking tumor immunity and angiogenesis. Curr Opin Immunol 2017, 45, 8-15.
56. Roberts, A. A.; Amano, M.; Felten, C.; Galvan, M.; Sulur, G.; Pinter-Brown, L.; Dobbeling, U.; Burg, G.; Said, J.; Baum, L. G., Galectin-1-mediated apoptosis in mycosis fungoides: The roles of CD7 and cell surface glycosylation. Modern Pathology 2003, 16, (6), 543-551.
57. Tsuboi, S.; Sutoh, M.; Hatakeyama, S.; Hiraoka, N.; Habuchi, T.; Horikawa, Y.; Hashimoto, Y.; Yoneyama, T.; Mori, K.; Koie, T.; Nakamura, T.; Saitoh, H.; Yamaya, K.; Funyu, T.; Fukuda, M.; Ohyama, C., A novel strategy for evasion of NK cell immunity by tumours expressing core2 O-glycans. EMBO J 2011, 30, (15), 3173-85.
58. Tsuboi, S.; Hatakeyama, S.; Ohyama, C.; Fukuda, M., Two opposing roles of O-glycans in tumor metastasis. Trends in molecular medicine 2012, 18, (4), 224-32.
59. Tsuboi, S., Immunosuppressive Functions of Core2 O-Glycans against NK Immunity. Trends in Glycoscience and Glycotechnology 2013, 25, (143), 117-123.
60. Tsuboi, S., Roles of Glycans in Immune Evasion from NK Immunity. In Sugar Chains: Decoding the Functions of Glycans, Suzuki, T.; Ohtsubo, K.; Taniguchi, N., Eds. Springer Japan: Tokyo, 2015; pp 177-188.
61. Suzuki, Y.; Sutoh, M.; Hatakeyama, S.; Mori, K.; Yamamoto, H.; Koie, T.; Saitoh, H.; Yamaya, K.; Funyu, T.; Habuchi, T.; Arai, Y.; Fukuda, M.; Ohyama, C.; Tsuboi, S., MUC1 carrying core 2 O-glycans functions as a molecular shield against NK cell attack, promoting bladder tumor metastasis. International journal of oncology 2012, 40, (6), 1831-1838.
62. Okamoto, T.; Yoneyama, M. S.; Hatakeyama, S.; Mori, K.; Yamamoto, H.; Koie, T.; Saitoh, H.; Yamaya, K.; Funyu, T.; Fukuda, M.; Ohyama, C.; Tsuboi, S., Core2 O-glycan-expressing prostate cancer cells are resistant to NK cell immunity. Mol Med Rep 2013, 7, (2), 359-64.
63. Van Rinsum, J.; Smets, L. A.; Van Rooy, H.; Van den Eijnden, D. H., Specific inhibition of human natural killer cell-mediated cytotoxicity by sialic acid and sialo-oligosaccharides. Int J Cancer 1986, 38, (6), 915-22.
64. Ogata, S.; Maimonis, P. J.; Itzkowitz, S. H., Mucins Bearing the Cancer-Associated Sialosyl-Tn Antigen Mediate Inhibition of Natural-Killer-Cell Cytotoxicity. Cancer Res 1992, 52, (17), 4741-4746.
65. Hudak, J. E.; Canham, S. M.; Bertozzi, C. R., Glycocalyx engineering reveals a Siglec-based mechanism for NK cell immunoevasion. Nature chemical biology 2014, 10, (1), 69-75.
66. Recchi, M. A.; Hebbar, M.; Hornez, L.; Harduin-Lepers, A.; Peyrat, J. P.;
Delannoy, P., Multiplex reverse transcription polymerase chain reaction assessment of sialyltransferase expression in human breast cancer. Cancer Res 1998, 58, (18), 4066-70.
67. Lise, M.; Belluco, C.; Perera, S. P.; Patel, R.; Thomas, P.; Ganguly, A., Clinical correlations of alpha2,6-sialyltransferase expression in colorectal cancer patients. Hybridoma 2000, 19, (4), 281-6.
68. Moriwaki, K.; Noda, K.; Furukawa, Y.; Ohshima, K.; Uchiyama, A.; Nakagawa, T.; Taniguchi, N.; Daigo, Y.; Nakamura, Y.; Hayashi, N.; Miyoshi, E., Deficiency of GMDS Leads to Escape from NK Cell-Mediated Tumor Surveillance Through Modulation of TRAIL Signaling. Gastroenterology 2009, 137, (1), 188-198.
69. Iwata, T.; Nishiyama, N.; Nagano, K.; Izumi, N.; Tsukioka, T.; Chung, K.; Hanada, S.; Inoue, K.; Kaji, M.; Suehiro, S., Preoperative serum value of sialyl Lewis X predicts pathological nodal extension and survival in patients with surgically treated small cell lung cancer. J Surg Oncol 2012, 105, (8), 818-24.
70. Mizuguchi, S.; Inoue, K.; Iwata, T.; Nishida, T.; Izumi, N.; Tsukioka, T.; Nishiyama, N.; Uenishi, T.; Suehiro, S., High serum concentrations of sialyl Lewis X predict multilevel N2 disease in non-small-cell lung cancer. Annals of Surgical Oncology 2006, 13, (7), 1010-1018.
71. Mizuguchi, S.; Nishiyama, N.; Iwata, T.; Nishida, T.; Izumi, N.; Tsukioka, T.; Inoue, K.; Kameyama, M.; Suehiro, S., Clinical value of serum cytokeratin 19 fragment and sialyl-Lewis x in non-small cell lung cancer. Ann Thorac Surg 2007, 83, (1), 216-21.
72. Mizuguchi, S.; Nishiyama, N.; Iwata, T.; Nishida, T.; Izumi, N.; Tsukioka, T.; Inoue, K.; Uenishi, T.; Wakasa, K.; Suehiro, S., Serum Sialyl Lewis(x) and cytokeratin 19 fragment as predictive factors for recurrence in patients with stage I non-small cell lung cancer. Lung Cancer 2007, 58, (3), 369-375.
73. Carbone, D. P.; Salmon, J. S.; Billheimer, D.; Chen, H.; Sandler, A.; Roder, H.; Roder, J.; Tsypin, M.; Herbst, R. S.; Tsao, A. S.; Tran, H. T.; Dang, T. P., VeriStrat (R) classifier for survival and time to progression in non-small cell lung cancer (NSCLC) patients treated with erlotinib and bevacizumab. Lung Cancer 2010, 69, (3), 337-340.
74. Akerley, W. L.; Arnaud, A. M.; Reddy, B.; Page, R. D., Impact of a multivariate serum-based proteomic test on physician treatment recommendations for advanced non-small-cell lung cancer. Curr Med Res Opin 2017, 33, (6), 1091-1097.
75. Grossi, F.; Rijavec, E.; Genova, C.; Barletta, G.; Biello, F.; Maggioni, C.; Burrafato, G.; Sini, C.; Dal Bello, M. G.; Meyer, K.; Roder, J.; Roder, H.; Grigorieva, J., Serum proteomic test in advanced non-squamous non-small cell lung cancer treated in first line with standard chemotherapy. Br J Cancer 2017, 116, (1), 36-43.
76. Milan, E.; Lazzari, C.; Anand, S.; Floriani, I.; Torri, V.; Sorlini, C.; Gregorc, V.; Bachi, A., SAA1 is over-expressed in plasma of non small cell lung cancer patients with poor outcome after treatment with epidermal growth factor receptor tyrosine-kinase inhibitors. J Proteomics 2012, 76, 91-101.

### References Cited in the Bladder Cancer Section of Background and in Example 2

1. Siegel R L, Miller K D, Jemal A. Cancer statistics, 2016. C A Cancer J Clin. 2016; 66(1):7-30. Epub 2016/01/09. doi: 10.3322/caac.21332. PubMed PMID: 26742998.
2. Ries L A G, L. Y J, Keel G E, Eisner M P, Lin Y D, Horner M-J, et al. SEER Survival Monograph: Cancer Survival Among Adults: U.S. SEER Program, 1988-2001, Patient and Tumor Characteristics. National Cancer Institute, SEER Program, NIH Pub. No. 07-6215, Bethesda, Md. 2007.
3. Babjuk M, Burger M, Zigeuner R, Shariat S F, van Rhijn B W, Comperat E, et al. EAU guidelines on non-muscle-invasive urothelial carcinoma of the bladder: update 2013. Eur Urol. 2013; 64(4):639-53. Epub 2013/07/06. doi: 10.1016/j.eururo.2013.06.003. PubMed PMID: 23827737.
4. Goodison S, Rosser C J, Urquidi V. Bladder cancer detection and monitoring: assessment of urine- and blood-based marker tests. Mol Diagn Ther. 2013; 17(2):71-84. Epub 2013/03/13. doi: 10.1007/s40291-013-0023-x. PubMed PMID: 23479428; PubMed Central PMCID: PMCPMC3627848.
5. Kehinde E O, Al-Mulla F, Kapila K, Anim J T. Comparison of the sensitivity and specificity of urine cytology, urinary nuclear matrix protein-22 and multitarget fluorescence in situ hybridization assay in the detection of bladder cancer. Scand J Urol Nephrol. 2011; 45(2):113-21. Epub 2010/11/26. doi: 10.3109/00365599.2010.533694. PubMed PMID: 21091091.
6. Hafeez S, Huddart R. Advances in bladder cancer imaging. Bmc Medicine. 2013; 11. doi: Artn 104 10.1186/1741-7015-11-104. PubMed PMID: WOS:000318442400002.
7. Mitra A P, Hansel D E, Cote R J. Prognostic value of cell-cycle regulation biomarkers in bladder cancer. Semin Oncol. 2012; 39(5):524-33. Epub 2012/10/09. doi: 10.1053/j.seminoncol.2012.08.008. PubMed PMID: 23040249; PubMed Central PMCID: PMCPMC3478886.
8. Yun S J, Jeong P, Kim W T, Kim T H, Lee Y S, Song P H, et al. Cell-free microRNAs in urine as diagnostic and prognostic biomarkers of bladder cancer. Int J Oncol. 2012; 41(5):1871-8. doi: 10.3892/ijo.2012.1622. PubMed PMID: WOS:000310114100038.
9. Scheffer A R, Holdenrieder S, Kristiansen G, von Ruecker A, Muller S C, Ellinger J. Circulating microRNAs in serum: novel biomarkers for patients with bladder cancer?World J Urol. 2014; 32(2):353-8. Epub 2012/12/26. doi: 10.1007/s00345-012-1010-2. PubMed PMID: 23266581.
10. Nagata M, Muto S, Horie S. Molecular Biomarkers in Bladder Cancer: Novel Potential Indicators of Prognosis and Treatment Outcomes. Dis Markers. 2016; 2016:8205836. Epub 2016/03/01. doi: 10.1155/2016/8205836. PubMed PMID: 26924873; PubMed Central PMCID: PMCPMC4746343.
11. Azevedo R, Peixoto A, Gaiteiro C, Fernandes E, Neves M, Lima L, et al. Over forty years of bladder cancer glycobiology: Where do glycans stand facing precision oncology? Oncotarget. 2017; 8(53):91734-64. doi: 10. 18632/oncotarget. 19433. PubMed PMID: WOS:000414175500106.
12. Hegele A, Mecklenburg V, Varga Z, Olbert P, Hofmann R, Barth P. CA19.9 and CEA in transitional cell carcinoma of the bladder: serological and immunohistochemical findings. Anticancer research. 2010; 30(12):5195-200. Epub 2010/12/29. PubMed PMID: 21187512.
13. Roy S, Dasgupta A, Kar K. Comparison of Urinary and Serum C A 19-9 as Markers of Early Stage Urothelial Carcinoma. Int Braz J Urol. 2013; 39(5):631-8. doi: 10.1590/S1677-5538.Ibju.2013.05.04. PubMed PMID: WOS:000327883800004.
14. Oikawa M, Hatakeyama S, Yoneyma T, Tobisawa Y, Narita T, Yamamoto H, et al. Significance of Serum N-glycan Profiling as a Diagnostic Biomarker in Urothelial Carcinoma. (Article in Press). Eur Urol Focus. 2016. doi: http://dx.doi.org/10.10169j,euf.2016.11.004.
15. Tanaka T, Yoneyama T, Noro D, Imanishi K, Kojima Y, Hatakeyama S, et al. Aberrant N-Glycosylation Profile of Serum Immunoglobulins is a Diagnostic Biomarker of Urothelial Carcinomas. International journal of molecular sciences. 2017; 18(12). doi: ARTN 2632
   10.3390/ijms18122632. PubMed PMID: WOS:000418896700136.
16. Varki A, Kannagi R, Toole B. Glycosylation Changes in Cancer. In: Varki A, Cummings R D, Esko J D, Freeze H H, Stanley P, Bertozzi C R, et al., editors. Essentials of Glycobiology. 2nd ed. Cold Spring Harbor, New York: Cold Spring Harbor Laboratory Press; 2009.
17. van Kooyk Y, Rabinovich G A. Protein-glycan interactions in the control of innate and adaptive immune responses. Nat Immunol. 2008; 9(6):593-601. Epub 2008/05/21. doi: 10.1038/ni.f.203. PubMed PMID: 18490910.
18. Borges C R, Rehder D S, Boffetta P. Multiplexed surrogate analysis of glycotransferase activity in whole biospecimens. Analytical chemistry. 2013; 85(5):2927-36. Epub 2013/02/02. doi: 10.1021/ac3035579. PubMed PMID: 23368525.
19. Zaare S, Aguilar J S, Hu Y, Ferdosi S, Borges C R. Glycan Node Analysis: A Bottom-up Approach to Glycomics. J Vis Exp. 2016; 111:e53961. doi: 10.3791/53961.
20. Hu Y, Borges C R. A spin column-free approach to sodium hydroxide-based glycan permethylation. Analyst. 2017; 142(15):2748-59. Epub 2017/06/22. doi: 10.1039/c7an00396j. PubMed PMID: 28635997.
21. Ferdosi S, Rehder D S, Maranian P, Castle E P, Ho T H, Pass H I, et al. Stage Dependence, Cell-Origin Independence, and Prognostic Capacity of Serum Glycan Fucosylation, beta1-4 Branching, beta1-6 Branching, and alpha2-6 Sialylation in Cancer. Journal of proteome research. 2018; 17(1):543-58. Epub 2017/11/14. doi: 10.1021/acs.jproteome.7b00672. PubMed PMID: 29129073.
22. Callewaert N, Van Vlierberghe H, Van Hecke A, Laroy W, Delanghe J, Contreras R. Noninvasive diagnosis of liver cirrhosis using DNA sequencer-based total serum protein glycomics. Nature Medicine. 2004; 10(4):429-34. doi: 10.1038/nm1006. PubMed PMID: ISI:000220587000037.
23. Vanderschaeghe D, Laroy W, Sablon E, Halfon P, Van Hecke A, Delanghe J, et al. GlycoFibroTest Is a Highly Performant Liver Fibrosis Biomarker Derived from DNA Sequencer-based Serum Protein Glycomics. Molecular & Cellular Proteomics. 2009; 8(5):986-94. doi: 10.1074/mcp.M800470-MCP200. PubMed PMID: ISI:000266116900010.
24. Miyahara K, Nouso K, Saito S, Hiraoka S, Harada K, Takahashi S, et al. Serum glycan markers for evaluation of disease activity and prediction of clinical course in patients with ulcerative colitis. PloS one. 2013; 8(10):e74861. Epub 2013/10/12. doi: 10.1371/journal.pone.0074861. PubMed PMID: 24116015; PubMed Central PMCID: PMC3792068.
25. Ansar W, Ghosh S. C-reactive protein and the biology of disease. Immunol Res. 2013; 56(1):131-42. Epub 2013/02/02. doi: 10.1007/s12026-013-8384-0. PubMed PMID: 23371836.
26. Eggers H, Seidel C, Schrader A J, Lehmann R, Wegener G, Kuczyk M A, et al. Serum C-reactive protein: a prognostic factor in metastatic urothelial cancer of the bladder. Med Oncol. 2013; 30(4):705. Epub 2013/09/06. doi: 10.1007/s12032-013-0705-6. PubMed PMID: 24005810.
27. Shrotriya S, Walsh D, Bennani-Baiti N, Thomas S, Lorton C. C-Reactive Protein Is an Important Biomarker for Prognosis Tumor Recurrence and Treatment Response in Adult Solid Tumors: A Systematic Review. PloS one. 2015; 10(12). doi: ARTN e0143080 10. 1371/journal.pone.0143080. PubMed PMID: WOS:000367510500004.
28. Zhou L, Cai X, Liu Q, Jian Z Y, Li H, Wang K J. Prognostic Role of C-Reactive Protein In Urological Cancers: A Meta-Analysis. Scientific reports. 2015; 5:12733. Epub 2015/08/04. doi: 10.1038/srep12733. PubMed PMID: 26235332; PubMed Central PMCID: PMCPMC4522672.
29. Mbeutcha A, Shariat S F, Rieken M, Rink M, Xylinas E, Seitz C, et al. Prognostic significance of markers of systemic inflammatory response in patients with non muscle-invasive bladder cancer. Urol Oncol-Semin Ori. 2016; 34(11). doi: 10.1016/j.urolonc.2016.05.013. PubMed PMID: WOS:000392642100007.
30. Ho T H, Nateras R N, Yan H, Park J G, Jensen S, Borges C, et al. A Multidisciplinary Biospecimen Bank of Renal Cell Carcinomas Compatible with Discovery Platforms at Mayo Clinic, Scottsdale, Ariz. PloS one. 2015; 10(7):e0132831. Epub 2015/07/17. doi: 10.1371/journal.pone.0132831. PubMed PMID: 26181416; PubMed Central PMCID: PMC4504486.
31. Anderson N L, Anderson N G. The human plasma proteome: history, character, and
diagnostic prospects. Molecular & cellular proteomics: MCP. 2002; 1(11):845-67. Epub 2002/12/19. PubMed PMID: 12488461.
32. Baker E S, Liu T, Petyuk V A, Burnum-Johnson K E, Ibrahim Y M, Anderson G A, et al. Mass spectrometry for translational proteomics: progress and clinical implications. Genome medicine. 2012; 4(8):63. Epub 2012/09/05. doi: 10.1186/gm364. PubMed PMID: 22943415; PubMed Central PMCID: PMC3580401.
33. Gryska K, Slupianek A, Laciak M, Gorny A, Mackiewicz K, Baumann H, et al. Inflammatory cytokines controlling branching of N-heteroglycans of acute phase protein. Adv Exp Med Biol. 1995; 376:239-45. Epub 1995/01/01. PubMed PMID: 8597254.
34. Narisada M, Kawamoto S, Kuwamoto K, Moriwaki K, Nakagawa T, Matsumoto H, et al. Identification of an inducible factor secreted by pancreatic cancer cell lines that stimulates the production of fucosylated haptoglobin in hepatoma cells. Biochem Bioph Res Co. 2008; 377(3):792-6. doi: DOI 10.1016/j.bbrc.2008.10.061. PubMed PMID: ISI:000261458900011.
35. Arnold J N, Saldova R, Hamid U M, Rudd P M. Evaluation of the serum N-linked glycome for the diagnosis of cancer and chronic inflammation. Proteomics. 2008; 8(16):3284-93. Epub 2008/07/23. doi: 10.1002/pmic.200800163. PubMed PMID: 18646009.
36. Saldova R, Wormald M R, Dwek R A, Rudd P M. Glycosylation changes on serum glycoproteins in ovarian cancer may contribute to disease pathogenesis. Dis Markers. 2008; 25(4-5):219-32. PubMed PMID: ISI:000263028900004.
37. Sarrats A, Saldova R, Pla E, Fort E, Harvey D J, Struwe W B, et al. Glycosylation of liver acute-phase proteins in pancreatic cancer and chronic pancreatitis. Proteomics Clinical applications. 2010; 4(4):432-48. Epub 2010/12/08. doi: 10.1002/prca.200900150. PubMed PMID: 21137062.
38. Vasseur J A, Goetz J A, Alley W R, Jr., Novotny M V. Smoking and lung cancer-induced changes in N-glycosylation of blood serum proteins. Glycobiology. 2012; 22(12):1684-708. Epub 2012/07/12. doi: 10.1093/glycob/cws108. PubMed PMID: 22781126; PubMed Central PMCID: PMC3486013.
39. Hulsmeier A J, Tobler M, Burda P, Hennet T. Glycosylation site occupancy in health, congenital disorder of glycosylation and fatty liver disease. Scientific reports. 2016; 6:33927. Epub 2016/10/12. doi: 10.1038/srep33927. PubMed PMID: 27725718; PubMed Central PMCID: PMCPMC5057071.
40. Hamfjord J, Saldova R, Stockmann H, Sandhu V, Bowitz Lothe I M, Buanes T, et al. Serum N-Glycome Characterization in Patients with Resectable Periampullary Adenocarcinoma. Journal of proteome research. 2015; 14(12):5144-56. Epub 2015/10/31. doi: 10.1021/acs.jproteome.5b00395. PubMed PMID: 26515733.
41. Jansen B C, Bondt A, Reiding K R, Lonardi E, de Jong C J, Falck D, et al. Pregnancy-associated serum N-glycome changes studied by high-throughput MALDI-TOF-M S. Scientific reports. 2016; 6:23296. Epub 2016/04/15. doi: 10.1038/srep23296. PubMed PMID: 27075729; PubMed Central PMCID: PMCPMC4831011.
42. Mantovani A, Allavena P, Sica A, Balkwill F. Cancer-related inflammation. Nature. 2008; 454(7203):436-44. Epub 2008/07/25. doi: 10.1038/nature07205. PubMed PMID: 18650914.
43. Hanahan D, Weinberg R A. Hallmarks of cancer: the next generation. Cell. 2011; 144(5):646-74. Epub 2011/03/08. doi: 10.1016/j.cell.2011.02.013. PubMed PMID: 21376230.
44. Wang D, DuBois R N. Immunosuppression associated with chronic inflammation in the tumor microenvironment. Carcinogenesis. 2015; 36(10):1085-93. Epub 2015/09/12. doi: 10.1093/carcin/bgv123. PubMed PMID: 26354776; PubMed Central PMCID: PMC5006153.
45. Wysoczynski M, Ratajczak M Z. Lung cancer secreted microvesicles: underappreciated modulators of microenvironment in expanding tumors. International journal of cancer Journal international du cancer. 2009; 125(7):1595-603. Epub 2009/05/23. doi: 10.1002/ijc.24479. PubMed PMID: 19462451; PubMed Central PMCID: PMC2769262.
46. Martins V R, Dias M S, Hainaut P. Tumor-cell-derived microvesicles as carriers of molecular information in cancer. Curr Opin Oncol. 2013; 25(1):66-75. doi: 10.1097/CCO.0b013e32835b7c81. PubMed PMID: ISI:000311975000012.
47. Fontana S, Saieva L, Taverna S, Alessandro R. Contribution of proteomics to understanding the role of tumor-derived exosomes in cancer progression: state of the art and new perspectives. Proteomics. 2013; 13(10-11): 1581-94. Epub 2013/02/13. doi: 10.1002/pmic.201200398. PubMed PMID: 23401131.
48. Rabinovich G A, Conejo-Garcia J R. Shaping the Immune Landscape in Cancer by Galectin-Driven Regulatory Pathways. J Mol Biol. 2016; 428(16):3266-81. Epub 2016/04/04. doi: 10.1016/j.jmb.2016.03.021. PubMed PMID: 27038510.
49. Sethi M K, Hancock W S, Fanayan S. Identifying N-Glycan Biomarkers in Colorectal Cancer by Mass Spectrometry. Accounts of chemical research. 2016; 49(10):2099-106. Epub 2016/10/19. doi: 10.1021/acs.accounts.6b00193. PubMed PMID: 27653471.
50. Roberts A A, Amano M, Felten C, Galvan M, Sulur G, Pinter-Brown L, et al. Galectin-1-mediated apoptosis in mycosis fungoides: The roles of CD7 and cell surface glycosylation. Modem Pathol. 2003; 16(6):543-51. doi: 10.1097/01.Mp.0000071840.84469.06. PubMed PMID: ISI:000183656200004.
51. Cagnoni A J, Perez Saez J M, Rabinovich G A, Marino K V. Turning-Off Signaling by Siglecs, Selectins, and Galectins: Chemical Inhibition of Glycan-Dependent Interactions in Cancer. Front Oncol. 2016; 6:109. Epub 2016/06/01. doi: 10.3389/fonc.2016.00109. PubMed PMID: 27242953; PubMed Central PMCID: PMC4865499.
52. Mendez-Huergo S P, Blidner A G, Rabinovich G A. Galectins: emerging regulatory checkpoints linking tumor immunity and angiogenesis. Current opinion in immunology. 2017; 45:8-15. Epub 2017/01/15. doi: 10.1016/j.coi.2016.12.003. PubMed PMID: 28088061.
53. Tsuboi S, Sutoh M, Hatakeyama S, Hiraoka N, Habuchi T, Horikawa Y, et al. A novel strategy for evasion of N K cell immunity by tumours expressing core2 O-glycans. EMBO J. 2011; 30(15):3173-85. Epub 2011/06/30. doi: 10.1038/emboj.2011.215. PubMed PMID: 21712812; PubMed Central PMCID: PMC3160189.
54. Tsuboi S, Hatakeyama S, Ohyama C, Fukuda M. Two opposing roles of O-glycans in tumor metastasis. Trends Mol Med. 2012; 18(4):224-32. Epub 2012/03/20. doi: 10.1016/j.molmed.2012.02.001. PubMed PMID: 22425488; PubMed Central PMCID: PMC3356160.
55. Tsuboi S. Immunosuppressive Functions of Core2 O-Glycans against N K Immunity. Trends Glycosci Glyc. 2013; 25(143):117-23. doi: 10.4052/tigg.25.117. PubMed PMID: ISI:000209502100002.
56. Tsuboi S. Roles of Glycans in Immune Evasion from N K Immunity. In: Suzuki T, Ohtsubo K, Taniguchi N, editors. Sugar Chains: Decoding the Functions of Glycans. Tokyo: Springer Japan; 2015. p. 177-88.
57. Suzuki Y, Sutoh M, Hatakeyama S, Mori K, Yamamoto H, Koie T, et al. MUC1 carrying core 2 O-glycans functions as a molecular shield against NK cell attack, promoting bladder tumor metastasis. Int J Oncol. 2012; 40(6):1831-8. doi: 10.3892/ijo.2012.1411. PubMed PMID: ISI:000303699900012.
58. Okamoto T, Yoneyama M S, Hatakeyama S, Mori K, Yamamoto H, Koie T, et al. Core2 O-glycan-expressing prostate cancer cells are resistant to N K cell immunity. Molecular medicine reports. 2013; 7(2):359-64. Epub 2012/11/21. doi: 10.3892/mmr.2012.1189. PubMed PMID: 23165940; PubMed Central PMCID: PMC3573034.
Ferdosi S et al., Behavior of blood plasma glycan features in bladder cancer. PloS One 2018, 13(7):e0201208.
Borges C R et al., Analytical chemistry 2013, 85(5):2927-2936.
Zaare S et al J Vis Exp 2016, 111:e53961. Hu Y and Borges C R, Analyst 2017, 142(15):2748-2759.

### References Cited in the Lung Cancer Section of Background and in Example 3

(1) Siegel, R. L.; Miller, K. D.; Jemal, A. Cancer statistics, 2019. Ca-Cancer J. Clin. 2019, 69, 7-34.
(2) Provencio, M.; Isla, D.; Sánchez, A.; Cantos, B. Inoperable stage III non-small cell lung cancer: Current treatment and role of vinorelbine. J. Thorac. Dis. 2011, 3, 197-204.
(3) National Lung Screening Trial Research Team. Reduced lung cancer mortality with low-dose computed tomographic screening. N. Engl. J. Med. 2011, 365, 395-409.
(4) Okamura, K.; Takayama, K.; Izumi, M.; Harada, T.; Furuyama, K.; Nakanishi, Y. Diagnostic value of CEA and CYFRA 21-1 tumor markers in primary lung cancer. Lung cancer 2013, 80, 45-49.
(5) Xu, Y.; Xu, L.; Qiu, M.; Wang, J.; Zhou, Q.; Xu, L.; Wang, J.; Yin, R. Prognostic value of serum cytokeratin 19 fragments (Cyfra 21-1) in patients with non-small cell lung cancer. Sci. Rep. 2015, 5, 9444.
(6) Arrieta, O.; Villarreal-Garza, C.; Martinez-Barrera, L.; Morales, M.; Dorantes-Gallareta, Y.; Pefia-Curiel, O.; Contreras-Reyes, S.; Macedo-Perez, E. O.; Alatorre-Alexander, J. Usefulness of serum carcinoembryonic antigen (CEA) in evaluating response to chemotherapy in patients with advanced non small-cell lung cancer: a prospective cohort study. BMC Cancer 2013, 13, 254.
(7) Wang, X.-B.; Li, J.; Han, Y. Prognostic significance of preoperative serum carcinoembryonic antigen in non-small cell lung cancer: a metaanalysis. Tumor Biol. 2014, 35, 10105-10110.
(8) Bianchi, F.; Nicassio, F.; Marzi, M.; Belloni, E.; Dall'Olio, V.; Bernard, L.; Pelosi, G.; Maisonneuve, P.; Veronesi, G.; Di Fiore, P. P. A serum circulating miRNA diagnostic test to identify asymptomatic high-risk individuals with early stage lung cancer. EMBO Mol. Med. 2011, 3, 495-503.
(9) Zheng, D.; Haddadin, S.; Wang, Y.; Gu, L.-Q.; Perry, M. C.; Freter, C. E.; Wang, M. X. Plasma microRNAs as novel biomarkers for early detection of lung cancer. Int. J. Clin. Exp. Pathol. 2011, 4, 575-586.
(10) Shen, J.; Todd, N. W.; Zhang, H.; Yu, L.; Lingxiao, X.; Mei, Y.; Guarnera, M.; Liao, J.; Chou, A.; Lu, C. L.; et al. Plasma microRNAs as potential biomarkers for non-small-cell lung cancer. Lab. Invest. 2011, 91, 579-587.
(11) Belinsky, S. A.; Klinge, D. M.; Dekker, J. D.; Smith, M. W.; Bocklage, T. J.; Gilliland, F. D.; Crowell, R. E.; Karp, D. D.; Stidley, C. A.; Picchi, M. A. Gene promoter methylation in plasma and sputum increases with lung cancer risk. Clin. Cancer Res. 2005, 11, 6505-6511.
(12) Balgkouranidou, I.; Chimonidou, M.; Milaki, G.; Tsarouxa, E.; Kakolyris, S.; Welch, D.; Georgoulias, V.; Lianidou, E. Breast cancer metastasis suppressor-1 promoter methylation in cell-free DNA provides prognostic information in non-small cell lung cancer. Br. J. Cancer 2014, 110, 2054-2062.
(13) Hou, J.-M.; Krebs, M.; Ward, T.; Sloane, R.; Priest, L.; Hughes, A.; Clack, G.; Ranson, M.; Blackhall, F.; Dive, C. Circulating tumor cells as a window on metastasis biology in lung cancer. Am. J. Pathol. 2011, 178, 989-996.
(14) Varki, A.; Kannagi, R.; Toole, B. P. Glycosylation Changes in Cancer. In Essentials of Glycobiology, 2nd ed.; Varki, A., Cummings, R. D., Esko, J. D., Freeze, H. H., Stanley, P., Bertozzi, C. R., Hart, G. W., Etzler, M. E., Eds.; Cold Spring Harbor Laboratory Press: Cold Spring Harbor, N.Y., 2009; pp 617-632.
(15) Ruhaak, L. R.; Miyamoto, S.; Lebrilla, C. B. Developments in the identification of glycan biomarkers for the detection of cancer. Mol. Cell. Proteomics 2013, 12, 846-855.
(16) Borges, C. R.; Rehder, D. S.; Boffetta, P. Multiplexed surrogate analysis of glycotransferase activity in whole biospecimens. Anal. Chem. 2013, 85, 2927-2936.
(17) Zaare, S.; Aguilar, J. S.; Hu, Y. M.; Ferdosi, S.; Borges, C. R. Glycan Node Analysis: A Bottom-up Approach to Glycomics. J. Visualized Exp. 2016, 111, No. e53961.
(18) Hu, Y.; Borges, C. R. A spin column-free approach to sodium hydroxide-based glycan permethylation. Analyst 2017, 142, 2748-2759.
(19) Ferdosi, S.; Rehder, D. S.; Maranian, P.; Castle, E. P.; Ho, T. H.; Pass, H. I.; Cramer, D. W.; Anderson, K. S.; Fu, L.; Cole, D. E. C.; Le, T.; Wu, X.; Borges, C. R. Stage Dependence, Cell-Origin Independence, and Prognostic Capacity of Serum Glycan Fucosylation, β1-4 Branching, β1-6 Branching, and α2-6 Sialylation in Cancer. J. Proteome Res. 2018, 17, 543-558.
(20) Ferdosi, S.; Ho, T. H.; Castle, E. P.; Stanton, M. L.; Borges, C. R. Behavior of blood plasma glycan features in bladder cancer. PLoS One 2018, 13, No. e0201208.
(21) Gasperino, J. Gender is a risk factor for lung cancer. Med. Hypotheses 2011, 76, 328-331.
(22) Osann, K. E.; Anton-Culver, H.; Kurosaki, T.; Taylor, T. Sex differences in lung-cancer risk associated with cigarette smoking. Int. J. Cancer 1993, 54, 44-48.
(23) Risch, H. A.; Howe, G. R.; Jain, M.; Burch, J. D.; Holowaty, E. J.; Miller, A. B. Are female smokers at higher risk for lung cancer than male smokers? A case-control analysis by histologic type. Am. J. Epidemiol. 1993, 138, 281-293.
(24) Pope, M.; Ashley, M.; Ferrence, R. The carcinogenic and toxic effects of tobacco smoke: are women particularly susceptible? J. Gender-Specific Med. 1999, 2, 45-51.
(25) Wakelee, H. A.; Chang, E. T.; Gomez, S. L.; Keegan, T. H.; Feskanich, D.; Clarke, C. A.; Holmberg, L.; Yong, L. C.; Kolonel, L. N.; Gould, M. K.; et al. Lung cancer incidence in never-smokers. J. Clin. Oncol. 2007, 25, 472-478.
(26) Stticker, I.; Martin, D.; Neri, M.; Laurent-Puig, P.; Blons, H.; Antoine, M.; Guiochon-Mantel, A.; Brailly-Tabard, S.; Canonico, M.; Wislez, M.; et al. Women Epidemiology Lung Cancer (WELCA) study: reproductive, hormonal, occupational risk factors and biobank. BMC Public Health 2017, 17, 324.
(27) Wang, B.-Y.; Huang, J.-Y.; Cheng, C.-Y.; Lin, C.-H.; Ko, J.-L.; Liaw, Y.-P. Lung cancer and prognosis in Taiwan: a population-based cancer registry. J. Thorac. Oncol. 2013, 8, 1128-1135.
(28) Knežević, A.; Gornik, O.; Polašek, O.; Pučić, M.; Redžić, I.; Novokmet, M.; Rudd, P. M.; Wright, A. F.; Campbell, H.; Rudan, I.; Lauc, G. Effects of aging, body mass index, plasma lipid profiles, and smoking on human plasma N-glycans. Glycobiology 2010, 20, 959-969.
(29) Reiding, K. R.; Ruhaak, L. R.; Uh, H.-W.; El Bouhaddani, S.; van den Akker, E. B.; Plomp, R.; McDonnell, L. A.; Houwing-Duistermaat, J. J.; Slagboom, P. E.; Beekman, M.; Wuhrer, M. Human plasma Nglycosylation as analyzed by matrix-assisted laser desorption/ionization-Fourier transform ion cyclotron resonance-MS associates with markers of inflammation and metabolic health. Mol. Cell. Proteomics 2017, 16, 228-242.
(30) Vasseur, J. A.; Goetz, J. A.; Alley, W. R., Jr; Novotny, M. V. Smoking and lung cancer-induced changes in N-glycosylation of blood serum proteins. Glycobiology 2012, 22, 1684-1708. (31) Hashimoto, S.; Asao, T.; Takahashi, J.; Yagihashi, Y.; Nishimura, T.; Saniabadi, A. R.; Poland, D. C.; van Dijk, W.; Kuwano, H.; Kochibe, N.; et al. al-Acid glycoprotein fucosylation as a marker of carcinoma progression and prognosis. Cancer 2004, 101, 2825-2836.
(32) Arnold, J. N.; Saldova, R.; Hamid, U. M. A.; Rudd, P. M. Evaluation of the serum N-linked glycome for the diagnosis of cancer and chronic inflammation. Proteomics 2008, 8, 3284-3293.
(33) Mizuguchi, S.; Inoue, K.; Iwata, T.; Nishida, T.; Izumi, N.; Tsukioka, T.; Nishiyama, N.; Uenishi, T.; Suehiro, S. High serum concentrations of Sialyl Lewisx predict multilevel N2 disease in non-small-cell lung cancer. Annals of surgical oncology 2006, 13, 1010-1018.
(34) Mizuguchi, S.; Nishiyama, N.; Iwata, T.; Nishida, T.; Izumi, N.; Tsukioka, T.; Inoue, K.; Kameyama, M.; Suehiro, S. Clinical value of serum cytokeratin 19 fragment and sialyl-Lewis x in non-small cell lung cancer. Annals of thoracic surgery 2007, 83, 216-221.
(35) Mizuguchi, S.; Nishiyama, N.; Iwata, T.; Nishida, T.; Izumi, N.; Tsukioka, T.; Inoue, K.; Uenishi, T.; Wakasa, K.; Suehiro, S. Serum Sialyl Lewisx and cytokeratin 19 fragment as predictive factors for recurrence in patients with stage I non-small cell lung cancer. Lung cancer 2007, 58, 369-375.
(36) Iwata, T.; Nishiyama, N.; Nagano, K.; Izumi, N.; Tsukioka, T.; Chung, K.; Hanada, S.; Inoue, K.; Kaji, M.; Suehiro, S. Preoperative serum value of sialyl Lewis X predicts pathological nodal extension and survival in patients with surgically treated small cell lung cancer. J. Surg. Oncol. 2012, 105, 818-824.
(37) Clement-Duchene, C.; Carnin, C.; Guillemin, F.; Martinet, Y. How accurate are physicians in the prediction of patient survival in advanced lung cancer? Oncologist 2010, 15, 782-789.
(38) El-Zayadi, A.-R. Heavy smoking and liver. World J. Gastroenterol 2006, 12, 6098-6101.
(39) Anderson, N. L.; Anderson, N. G. The human plasma proteome history, character, and diagnostic prospects. Mol. Cell. Proteomics 2002, 1, 845-867.
(40) Baker, E. S.; Liu, T.; Petyuk, V. A.; Burnum-Johnson, K. E.; Ibrahim, Y. M.; Anderson, G. A.; Smith, R. D. Mass spectrometry for translational proteomics: progress and clinical implications. Genome Med. 2012, 4, 63.
(41) Locher, C.; Debieuvre, D.; Coetmeur, D.; Goupil, F.; Molinier, O.; Collon, T.; Dayen, C.; Le Treut, J.; Asselain, B.; Martin, F.; et al. Major changes in lung cancer over the last ten years in France: the KBPCPHG studies. Lung Cancer 2013, 81, 32-38.
(42) Debieuvre, D.; Oster, J.-P.; Riou, R.; Berruchon, J.; Levy, A.; Mathieu, J.-P.; Dumont, P.; Leroy-Terquem, E.; Tizon-Couetil, V.; Martin, F.; et al. The new face of non-small-cell lung cancer in men: Results of two French prospective epidemiological studies conducted 10 years apart. Lung Cancer 2016, 91, 1-6.

While particular materials, formulations, operational sequences, process parameters, and end products have been set forth to describe and exemplify this invention, they are not intended to be limiting. Rather, it should be noted by those ordinarily skilled in the art that the written disclosures are exemplary only and that various other alternatives, adaptations, and modifications may be made within the scope of the present invention. Accordingly, the present invention is not limited to the specific embodiments illustrated herein but is limited only by the following claims.

What is claimed is:

1. A method of detecting altered glycan nodes in a sample from a patient having or being treated for cancer, suspected of having cancer or at risk to having cancer, the method comprising:
a. obtaining a sample from the patient, wherein the sample comprises glycans;
b. permethylating the sample comprising glycans;
c. hydrolyzing the product from step b;
d. reducing the product from step c;
e. acetylating the product from step d;
f. partially purifying the product from step e;
g. analyzing the product of step f using a substance identifying technique to detect altered glycan nodes in the sample.

2. The method of claim 1, wherein step (b) includes an initial substep of mixing the sample comprising glycans with a labeled chemical substance.

3. The method of claim 2, wherein the labeled chemical substance is heavy-labeled D-glucose, N-acetyl-D-[UL-¹³C₆]glucosamine, or combinations thereof.

4. The method of claim 1, wherein step (b) comprises liquid/liquid extraction.

5. The method of claim 1, wherein step (c) uses trifluoroacetic acid.

6. The method of claim 1, wherein step (d) uses a reducing agent selected from the group consisting of NaBH₄, NaBD₄ and a combination thereof.

7. The method of claim 1, wherein step (e) uses acetic anhydride.

8. The method of claim 1, wherein step (f) comprises liquid/liquid extraction.

9. The method of claim 1, wherein the substance identifying technique of step (g) is GC-MS.

10. A method of detecting terminal fucosylation, α2-6 sialylation, β1-4 branching, β1-6 branching, and outer-arm fucosylation in a sample from a patient having or being treated for cancer, suspected of having cancer or at risk to having cancer, the method comprising:
a. obtaining a sample from the patient, wherein the sample comprises glycans;
b. permethylating the sample comprising glycans;
c. hydrolyzing the product from step (b);
d. reducing the product from step (c);
e. acetylating the product from step (d);
f. purifying the product from step (e);
g. analyzing the product of step (f) using a substance identifying technique to terminal fucosylation, α2-6 sialylation, β1-4 branching, β1-6 branching, and outer-arm fucosylation in the sample.

11. The method of claim 10, wherein step (b) includes an initial substep of mixing the sample comprising glycans with a labeled chemical substance.

12. The method of claim 11, wherein the labeled chemical substance is heavy-labeled D-glucose, N-acetyl-D-[UL-¹³C₆]glucosamine, or combinations thereof.

13. The method of claim 10, wherein step (b) comprises liquid/liquid extraction.

14. The method of claim 10, wherein step (c) uses trifluoroacetic acid.

15. The method of claim 10, wherein step (d) uses a reducing agent selected from the group consisting of NaBH₄, NaBD₄ and a combination thereof.

16. The method of claim 10, wherein step (e) uses acetic anhydride.

17. The method of claim 10, wherein step (f) comprises liquid/liquid extraction.

18. The method of claim 10, wherein the substance identifying technique of step (g) is GC-MS.

19. A method of detecting α2-6 sialylation, β1-4 branching, β1-6 branching, and outer-arm fucosylation in a sample from a patient having or being treated for bladder cancer, suspected of having bladder cancer or at risk to having bladder cancer, the method comprising:
a. obtaining a sample from the patient, wherein the sample comprises glycans;
b. permethylating the sample comprising glycans;
c. hydrolyzing the product from step (b);
d. reducing the product from step (c);
e. acetylating the product from step (d);
f. purifying the product from step (e);
g. analyzing the product of step (f) using a substance identifying technique to terminal fucosylation, α2-6 sialylation, β1-4 branching, β1-6 branching, and outer-arm fucosylation in the sample.

20. The method of claim 19, wherein step (b) includes an initial substep of mixing the sample comprising glycans with a labeled chemical substance.

21. The method of claim 20, wherein the labeled chemical substance is heavy-labeled D-glucose, N-acetyl-D-[UL-¹³C₆]glucosamine, or combinations thereof.

22. The method of claim 19, wherein step (b) comprises liquid/liquid extraction.

23. The method of claim 19, wherein step (c) uses trifluoroacetic acid.

24. The method of claim 19, wherein step (d) uses a reducing agent selected from the group consisting of NaBH₄, NaBD₄ and a combination thereof.

25. The method of claim 19, wherein step (e) uses acetic anhydride.

26. The method of claim 19, wherein step (f) comprises liquid/liquid extraction.

27. The method of claim 19, wherein the substance identifying technique of step (g) is GC-MS.
